SEQUENCE LISTING (2 sequences)

SEQ ID NO: 1 - protein, 20 aa, Artificial Sequence
  activating peptide
  MOD_RES (1)..(1): AMIDATION
  MOD_RES (3)..(3): PHOSPHORYLATION
  SITE (9)..(10): linked by dPEG8
  MOD_RES (13)..(13): PHOSPHORYLATION
  MOD_RES (20)..(20): AMIDATION
LNYIDLDLVLSTYASINFQK

SEQ ID NO: 2 - protein, 19 aa, Artificial Sequence
  activating peptide
  MOD_RES (1)..(1): AMIDATION
  MOD_RES (3)..(3): PHOSPHORYLATION
  SITE (9)..(10): linked by dPEG8
  MOD_RES (13)..(13): PHOSPHORYLATION
  MOD_RES (19)..(19): AMIDATION
LNYAQLWHALTIYATIRRF

(19) United States
(12) Patent Application Publication
(10) Pub. No.: US 2022/0031695 A1
(43) Pub. Date: Feb. 3, 2022

(54) SHP2 INHIBITOR COMPOSITIONS FOR USE IN TREATING CANCER

(71) Applicants: Revolution Medicines, Inc., Redwood City, CA (US); The Regents of the University of California, Oakland, CA (US)

(72) Inventors: Robert J. NICHOLS, Redwood City, CA (US); Trever G. BIVONA, Oakland, CA (US); Dana NEEL, Oakland, CA (US)

(21) Appl. No.: 17/224,025

(22) Filed: Apr. 6, 2021

Related U.S. Application Data
(63) Continuation of application No. PCT/US2019/055036, filed on Oct. 7, 2019.
(60) Provisional application No. 62/742,787, filed on Oct. 8, 2018.

(51) Int. Cl. A61K 31/497; G01N 33/574; A61K 45/06

(52) U.S. Cl. CPC A61K 31/497; G01N 2800/7028; A61K 45/06; G01N 33/574

(57) ABSTRACT

The present disclosure provides methods of treating diseases or disorders using allosteric inhibitors of SHP2 and to methods and diagnostic tests for identifying subjects likely to respond to such allosteric inhibitors of SHP2. In particular, the present disclosure provides diagnostic and therapeutic uses related to certain Receptor Tyrosine Kinase (RTK) mutations that are indicative of sensitivity to allosteric SHP2 inhibitors.

### CROSS REFERENCE

This application claims priority to U.S. Provisional Application No. 62/742,787, filed Oct. 8, 2018, which is incorporated by reference herein in its entirety.

### FIELD OF THE INVENTION

The present disclosure relates to methods for the treatment of diseases or disorders (e.g., cancer) with inhibitors of the protein tyrosine phosphatase SHP2. Specifically, this invention is concerned with methods of treating diseases or disorders (such as cancer) in subjects that are identified as candidates for treatment with an allosteric SHP2 inhibitor.

### BACKGROUND OF THE INVENTION

Cancer remains one of the most deadly threats to human health. In the U.S., cancer affects nearly 1.3 million new patients each year, and is the second leading cause of death after heart disease, accounting for approximately 1 in 4 deaths (US20170204187). Many cancers are caused by constitutive or aberrant hyper-activation of receptor tyrosine kinases (RTKs). This malignant RTK activation arises through a variety of mechanisms, including somatic genetic alterations such as missense mutations, small insertions and deletions, copy number alterations, and chromosomal rearrangements in RTK genes. (Bergethon et al., 2012; Kohno et al., 2015; Li et al., 2012; Lynch et al., 2004; Paez et al., 2004; Pao and Hutchinson, 2012; Rikova et al., 2007; Stephens et al., 2004; Takeuchi et al., 2009; Vaishnavi et al., 2013) The latter class of genetic alterations comprise a clinically important group of cancer driver genes, prominent examples of which are fusions of anaplastic lymphoma kinase (ALK), ROS proto-oncogene 1 (ROS1), RET, NTRK1, NTRK2, and NTRK3 among other kinases, with various fusion partners. (Takeuchi et al., 2012) These gene rearrangements often lead to the generation of a chimeric protein with the non-kinase partner fused N-terminal to the RTK kinase domain (e.g. EML4-ALK, CD74-ROS1, SDC4-ROS1). While tyrosine kinase inhibitors (TKIs) are effective in many patients harboring cancers driven by these kinase fusions (e.g. crizotinib targeting ALK and ROS1 fusions), drug resistance remains a challenge that limits long-term patient survival. (Doebele et al., 2012; Engelman et al., 2007; Kobayashi et al., 2005; Lynch et al., 2004; Ou et al., 2016; Pao et al., 2005; Rotow and Bivona, 2017; Shaw et al., 2014; Shaw and Solomon, 2015; Yun et al., 2008) A better understanding of the mechanisms controlling the oncogenic signaling properties of these kinase fusion proteins is essential to identify complementary or alternative molecular strategies to enhance clinical outcomes.

The mechanisms by which a non-native N-terminal protein fused to the kinase domain of an RTK such as ROS1 cause kinase hyper-activation and cancer growth are partially understood. These mechanisms include overexpression of the C-terminal kinase as a result of the activity of the promoter of the N-terminal gene partner, constitutive oligomerization of the fusion kinase proteins, and release of kinase auto-inhibitory mechanisms. (Medves and Demoulin, 2012) A relatively poorly-understood aspect of the regulation of oncoprotein kinase fusions is the extent to which the subcellular membrane localization of a particular fusion oncoprotein may contribute to its oncogenic properties. This is a particularly relevant unanswered cell biological question, as many oncoprotein fusion kinases present in human cancers, such as ALK and ROS1 variants, often gain the subcellular localization signals of the N-terminal partner in the context of the native RTK kinase domain (e.g. EML4 in EML4-ALK, CD74 in CD74-ROS1); it is therefore plausible that abnormal subcellular localization could be an important feature of their aberrant and oncogenic properties. While evidence is emerging that in certain cases the N-terminal fusion partner can cause aberrant subcellular localization of the fusion RTK compared to the native RTK (e.g. EML4-ALK), whether differential subcellular localization is a more general feature of oncogenesis across different oncoprotein fusions and whether such differential subcellular localization might affect the oncogenic properties of each kinase fusion or responses to TKI treatment remains unclear. (Hrustanovic et al., 2015) Furthermore, it is unclear whether, and to what extent, the N-terminal fusion partner might play a role in rendering cancers driven by oncogenic tyrosine kinase fusions sensitive to treatment with cancer therapies including, e.g., SHP2 inhibitors.

### SUMMARY OF THE INVENTION

The present disclosure relates to methods of treating diseases or disorders (such as cancer) in certain subsets of subjects that are determined to be candidates for treatment with an allosteric SHP2 inhibitor. The present disclosure is based in part on the surprising discovery that the efficacy of SHP2 inhibitor treatment on cancers containing oncogenic tyrosine kinase fusions is, in some embodiments, linked not to the presence of a tyrosine kinase domain in a fusion, but rather to the fusion partner linked to the tyrosine kinase domain. Certain cancer cells containing oncogenic tyrosine kinase fusions are insensitive to SHP2 inhibition (e.g., CD74-ROS1), whereas other cancer cells containing fusions of the same tyrosine kinase domain, but different N-terminal fusion partners (e.g., SDC4-ROS1, SLC34A2-ROS1) are rendered sensitive to SHP2 inhibition due at least in part to the differential activation of the MAPK pathway by these different fusion proteins. Furthermore, the inventors have demonstrated herein that, in some embodiments, this differential MAPK activation may be driven by the subcellular localization of the tyrosine kinase fusion, which may be determined by the N-terminal protein fused to the kinase domain. In some embodiments, the present disclosure provides a method for identifying whether a subject has a cancer that is sensitive to SHP2 inhibition, the method comprising determining whether the cancer comprises a cell containing an oncogenic tyrosine kinase fusion that causes MAPK activation. In some embodiments, the oncogenic tyrosine kinase fusion is selected from a ROS1 fusion, an ALK fusion, a RET fusion, an NTRK1 fusion, an NTRK2 fusion, and an NTRK3 fusion. In some embodiments, the oncogenic tyrosine kinase fusion is a SDC4-ROS1 fusion or an SLC34A2-ROS1 fusion. In some embodiments, the oncogenic tyrosine kinase fusion is selected from a FIG-ROS1 fusion; a LRIG3-ROS1 fusion; an EZR-ROS1 fusion, and a TPM3-ROS1 fusion. In some embodiments, the oncogenic tyrosine kinase fusion is selected from an EML4-ALK fusion. In some embodiments, the MAPK activation is detected by measuring increased ERK phosphorylation. In some embodiments, determining whether the cancer cells contain an oncogenic tyrosine kinase fusion that causes MAPK activation is achieved by genotyping a cell or cells in a biological sample obtained from the patient. In some embodiments, the genotyping determines whether the cancer comprises a cell containing an oncogenic tyrosine kinase fusion selected from EML4-ALK, SDC4-ROS1 and SLC34A2-ROS1. In some embodiments, the oncogenic tyrosine kinase fusion protein causes MAPK activation. In some embodiments, the subject that has been identified as having a cancer that is sensitive to SHP2 inhibition according to the above method is treated with a SHP2 inhibitor. In some embodiments, the SHP2 inhibitor is selected from (i) NSC-87877; (ii) TN0155, (iii) a SHP2 inhibitor compound of any one of Formula I, of Formula II, of Formula III, of Formula I-V1, of Formula I-V2, of Formula I-W, of Formula I-X, of Formula I-Y, of Formula I-Z, of Formula IV, of Formula V, of Formula VI, of Formula IV-X, of Formula IV-Y, of Formula IV-Z, of Formula VII, of Formula VIII, of Formula IX, and of Formula X disclosed herein; (iv) Compound C; (v) a compound from Table 1, disclosed herein; (vi) a compound from Table 2, disclosed herein; and (vii) combinations thereof. In some embodiments, the SHP2 inhibitor is a pharmaceutically acceptable salt, prodrug, solvate, hydrate, tautomer, or isomer of any one or more of the SHP2 inhibitors of (i)-(vi) above, or a combination thereof. In some embodiments, the subject that has been identified as having a cancer that is sensitive to SHP2 inhibition according to the above method is treated with a SHP2 inhibitor in combination (e.g., as a combination therapy) with one or more other therapeutic agent (e.g., an inhibitor of a MAP kinase pathway or an anti-cancer therapeutic agent).

In some embodiments, the present disclosure provides a method for using a SHP2 inhibitor to treat a subject with a cancer, the method comprising the steps of: (i) determining whether the cancer comprises a cell that contains an oncogenic tyrosine kinase fusion that causes MAPK activation; and (ii) administering the SHP2 inhibitor to the patient if the cancer comprises a cell that contains an oncogenic tyrosine kinase fusion that causes MAPK activation. In some embodiments, the SHP2 inhibitor is selected from (i) NSC-87877; (ii) TN0155, (iii) a SHP2 inhibitor compound of any one of Formula I, of Formula II, of Formula III, of Formula I-V1, of Formula I-V2, of Formula I-W, of Formula I-X, of Formula I-Y, of Formula I-Z, of Formula IV, of Formula V, of Formula VI, of Formula IV-X, of Formula IV-Y, of Formula IV-Z, of Formula VII, of Formula VIII, of Formula IX, and of Formula X disclosed herein; (iv) Compound C; (v) a compound from Table 1, disclosed herein; (vi) a compound from Table 2, disclosed herein; and (vii) combinations thereof. In some embodiments, the patient that has be determined to have a cancer comprising a cell that contains an oncogenic tyrosine kinase fusion that causes MAPK activation according to the above method is treated with a SHP2 inhibitor in combination (e.g., as a combination therapy) with one or more other therapeutic agent (e.g., an inhibitor of a MAP kinase pathway or an anti-cancer therapeutic agent). In some embodiments, the oncogenic tyrosine kinase fusion is selected from a ROS1 fusions, an ALK fusion, a RET fusion, an NTRK1 fusion, an NTRK2 fusion, and an NTRK3 fusion. In some embodiments, the oncogenic tyrosine kinase fusion is a SDC4-ROS1 fusion or an SLC34A2-ROS1 fusion. In some embodiments, the oncogenic tyrosine kinase fusion is selected from a FIG-ROS1 fusion; a LRIG3-ROS1 fusion; an EZR-ROS1 fusion, and a TPM3-ROS1 fusion. In some embodiments, the oncogenic tyrosine kinase fusion is selected from an EML4-ALK fusion. In some embodiments, the MAPK activation is detected by measuring ERK phosphorylation. In some embodiments, determining whether the cancer cells contain an oncogenic tyrosine kinase fusion that causes MAPK activation is achieved by genotyping a cell or cells in a biological sample obtained from the patient. In some embodiments, the genotyping determines whether the cancer comprises a cell containing an oncogenic tyrosine kinase fusion selected from EML4-ALK, SDC4-ROS1 and SLC34A2-ROS1. In some embodiments, the oncogenic tyrosine kinase fusion protein causes MAPK activation. In some embodiments, if the determining step (i) in the above method determines that the cancer does not comprise a cell containing an oncogenic tyrosine kinase fusion that causes MAPK activation, then the method comprises administering a cancer therapy selected from chemotherapy, radiation therapy, and/or surgical tumor resection, but not administering a SHP2 inhibitor.

In some embodiments, the present disclosure provides a method for killing cancer cells with a SHP2 inhibitor, the method comprising the steps of: (i) determining whether the cancer cells contain an oncogenic tyrosine kinase fusion that causes MAPK activation; and (ii) contacting the cancer cells with the SHP2 inhibitor if the cancer cells contains an oncogenic tyrosine kinase fusion that causes MAPK activation. In some embodiments, the SHP2 inhibitor is selected from (i) NSC-87877; (ii) TN0155, (iii) a SHP2 inhibitor compound of any one of Formula I, of Formula II, of Formula III, of Formula I-V1, of Formula I-V2, of Formula I-W, of Formula I-X, of Formula I-Y, of Formula I-Z, of Formula IV, of Formula V, of Formula VI, of Formula IV-X, of Formula IV-Y, of Formula IV-Z, of Formula VII, of Formula VIII, of Formula IX, and of Formula X disclosed herein; (iv) Compound C; (v) a compound from Table 1, disclosed herein; (vi) a compound from Table 2, disclosed herein; and (vii) combinations thereof. In some embodiments, cancer cells that are determined to contain an oncogenic tyrosine kinase fusion that causes MAPK activation according to the above method are treated with a SHP2 inhibitor in combination (e.g., as a combination therapy) with one or more other therapeutic agent (e.g., an inhibitor of a MAP kinase pathway or an anti-cancer therapeutic agent). In some embodiments, the oncogenic tyrosine kinase fusion is selected from a ROS1 fusions, an ALK fusion, a RET fusion, an NTRK1 fusion, an NTRK2 fusion, and an NTRK3 fusion. In some embodiments, the oncogenic tyrosine kinase fusion is a SDC4-ROS1 fusion or an SLC34A2-ROS1 fusion. In some embodiments, the oncogenic tyrosine kinase fusion is selected from a FIG-ROS1 fusion; a LRIG3-ROS1 fusion; an EZR-ROS1 fusion, and a TPM3-ROS1 fusion. In some embodiments, the oncogenic tyrosine kinase fusion is selected from an EML4-ALK fusion. In some embodiments, the MAPK activation is detected by measuring increased ERK phosphorylation. In some embodiments, determining whether the cancer cells contain an oncogenic tyrosine kinase fusion that causes MAPK activation is achieved by genotyping a cell or cells in a biological sample obtained from the patient. In some embodiments, the genotyping determines whether the cancer comprises a cell containing an oncogenic tyrosine kinase fusion selected from EML4-ALK, SDC4-ROS1 and SLC34A2-ROS1. In some embodiments, the oncogenic tyrosine kinase fusion protein causes MAPK activation. In some embodiments, if the cancer cells are determined according to the above method to not contain an oncogenic tyrosine kinase fusion that causes MAPK activation, then the method comprises contacting the cancer cells with a cancer therapy selected from chemotherapy, radiation therapy, and/or surgical tumor resection, but not a SHP2 inhibitor in order to kill the cancer cells.

In some embodiments, the present disclosure provides a method for treating a patient with a SHP2 inhibitor, wherein the patient has cancer, the method comprising the steps of: (i) determining whether the patient has a SHP2-sensitive cancer by: (a) obtaining or having obtained a biological sample from the patient; and (b) performing or having performed an assay on the biological sample to determine if the patient has a tumor comprising a cell that contains an oncogenic tyrosine kinase fusion that causes MAPK activation; and (ii) administering the SHP2 inhibitor to the patient if the patient has a tumor comprising a cell containing an oncogenic tyrosine kinase fusion that causes MAPK activation. In some embodiments, the SHP2 inhibitor is selected from (i) NSC-87877; (ii) TN0155, (iii) of any one of Formula I, of Formula II, of Formula III, of Formula I-V1, of Formula I-V2, of Formula I-W, of Formula I-X, of Formula I-Y, of Formula I-Z, of Formula IV, of Formula V, of Formula VI, of Formula IV-X, of Formula IV-Y, of Formula IV-Z, of Formula VII, of Formula VIII, of Formula IX, and of Formula X disclosed herein; (iv) Compound C; (v) a compound from Table 1, disclosed herein; (vi) a compound from Table 2, disclosed herein; and (vii) combinations thereof. In some embodiments, the SHP2 inhibitor is administered in combination (e.g., as a combination therapy) with one or more other therapeutic agent (e.g., an inhibitor of a MAP kinase pathway or an anti-cancer therapeutic agent) to a patient that has been determined according to step (i)(b) of the above method to have a tumor comprising a cell containing an oncogenic tyrosine kinase fusion that causes MAPK. In some embodiments, the oncogenic tyrosine kinase fusion is selected from a ROS1 fusions, an ALK fusion, a RET fusion, an NTRK1 fusion, an NTRK2 fusion, and an NTRK3 fusion. In some embodiments, the oncogenic tyrosine kinase fusion is a SDC4-ROS1 fusion or an SLC34A2-ROS1 fusion. In some embodiments, the oncogenic tyrosine kinase fusion is selected from a FIG-ROS1 fusion; a LRIG3-ROS1 fusion; an EZR-ROS1 fusion, and a TPM3-ROS1 fusion. In some embodiments, the oncogenic tyrosine kinase fusion is selected from an EML4-ALK fusion. In some embodiments, the MAPK activation is detected by measuring increased ERK phosphorylation. In some embodiments, determining whether the cancer cells contain an oncogenic tyrosine kinase fusion that causes MAPK activation is achieved by genotyping a cell or cells in a biological sample obtained from the patient. In some embodiments, the genotyping determines whether the cancer comprises a cell containing an oncogenic tyrosine kinase fusion selected from EML4-ALK, SDC4-ROS1 and SLC34A2-ROS1. In some embodiments, the oncogenic tyrosine kinase fusion protein causes MAPK activation. In some embodiments, if at step (i)(b) of the above method it is determined that the patient does not have a tumor comprising a cell containing an oncogenic tyrosine kinase fusion that causes MAPK activation, then the method comprises administering to the patient a cancer therapy selected from chemotherapy, radiation therapy, and/or surgical tumor resection, wherein the cancer therapy does not comprise administration of a SHP2 inhibitor.

In some embodiments, the present disclosure provides a method for treating a subject having a tumor with a SHP2 inhibitor, the method comprising: determining whether a biological sample obtained from the subject contains an oncogenic tyrosine kinase fusion protein comprising a N-terminal fusion partner that causes the fusion protein to localize in an endosome; and administering to the subject an inhibitor of SHP2 if the biological sample contains an oncogenic tyrosine kinase fusion protein comprising a N-terminal fusion partner that causes the fusion protein to localize in an endosome. In some embodiments, the oncogenic tyrosine kinase fusion protein that localizes in the endosome causes MAPK activation in the endosome. In some embodiments, the SHP2 inhibitor is selected from (i) NSC-87877; (ii) TN0155, (iii) of any one of Formula I, of Formula II, of Formula III, of Formula I-V1, of Formula I-V2, of Formula I-W, of Formula I-X, of Formula I-Y, of Formula I-Z, of Formula IV, of Formula V, of Formula VI, of Formula IV-X, of Formula IV-Y, of Formula IV-Z, of Formula VII, of Formula VIII, of Formula IX, and of Formula X disclosed herein; (iv) Compound C; (v) a compound from Table 1, disclosed herein; (vi) a compound from Table 2, disclosed herein; and (vii) combinations thereof. In some embodiments, the SHP2 inhibitor is administered in combination (e.g., as a combination therapy) with one or more other therapeutic agent (e.g., an inhibitor of a MAP kinase pathway or an anti-cancer therapeutic agent) if the biological sample is determined according to the above method to contain an oncogenic tyrosine kinase fusion protein comprising a N-terminal fusion partner that causes the fusion protein to localize in an endosome. In some embodiments, the oncogenic tyrosine kinase fusion is selected from a ROS1 fusions, an ALK fusion, a RET fusion, an NTRK1 fusion, an NTRK2 fusion, and an NTRK3 fusion. In some embodiments, the oncogenic tyrosine kinase fusion is a SDC4-ROS1 fusion or an SLC34A2-ROS1 fusion. In some embodiments, the oncogenic tyrosine kinase fusion is selected from a FIG-ROS1 fusion; a LRIG3-ROS1 fusion; an EZR-ROS1 fusion, and a TPM3-ROS1 fusion. In some embodiments, the oncogenic tyrosine kinase fusion is selected from an EML4-ALK fusion. In some embodiments, the MAPK activation is detected by measuring increased ERK phosphorylation. In some embodiments, determining whether the cancer cells contain an oncogenic tyrosine kinase fusion that causes MAPK activation is achieved by genotyping a cell or cells in a biological sample obtained from the patient. In some embodiments, the genotyping determines whether the cancer comprises a cell containing an oncogenic tyrosine kinase fusion selected from EML4-ALK, SDC4-ROS1 and SLC34A2-ROS1. In some embodiments, the oncogenic tyrosine kinase fusion protein causes MAPK activation.

In some embodiments, any one of the methods disclosed herein may further comprise administering the SHP2 inhibitor in combination with one or more additional therapy. In some embodiments, any one of the methods disclosed herein may further comprise administering the SHP2 inhibitor in combination with one or more additional therapy selected from a chemotherapy, immunotherapy, radiation therapy, and surgical tumor resection.

It is to be understood that one, some, or all of the properties of the various embodiments described herein may be combined to form other embodiments of the present invention. These and other aspects of the invention will become apparent to one of skill in the art.

### BRIEF DESCRIPTION OF THE DRAWINGS

FIG. 1. ROS1 fusion partners dictate differential activation of downstream pathways. (FIG. 1A) Diagram of the commonly occurring ROS1 fusion oncoproteins, which were studied here. Pink denotes a transmembrane domain. (FIG. 1B) Topological arrangement of ROS1 fusions based on CCTOP computational analysis. (Dobson et al., 2015a) (FIG. 1C) Immunoblot analysis of 293T cells transiently transfected for 48 h with GFP, SDC4-ROS1, CD74-ROS1, or SLC34A2-ROS1, with 5 h serum starvation. (FIGS. 1D-1G) Immunoblot analysis of patient-derived cell lines expressing (FIG. 1D) SDC4-ROS1, (FIG. 1E) SLC34A2-ROS1, or (FIGS. 1F-1G) CD74-ROS1 with siRNA-mediated knockdown of ROS1 (55 h after transfection). Data shown in (FIGS. 1C-1G) are representative of ≥3 independent experiments.

FIG. 2 MAPK pathway signaling is necessary and sufficient for survival of SDC4-ROS1-positive and SLC34A2-ROS1-positive lines, but not a CD74-ROS1 positive line. (FIGS. 2A-2C) Crystal violet quantification of ROS1 fusion-positive patient-derived cell lines (FIG. 2A) HCC78, (FIG. 2B) CUTO-2 and (FIG. 2C) CUTO-23, expressing empty vector or constitutively active MEK-DD, treated with DMSO or a dose-response of the ROS inhibitor crizotinib for 6 days. (FIG. 2D) Crystal violet quantification of HCC78 (SLC34A2-ROS1), CUTO-2 (SDC4-ROS1), CUTO-23 (CD74-ROS1), and CUTO-33 (CD74-ROS1) cell lines treated with DMSO or a dose-response of the SHP2 inhibitor RMC-4550 for 6 days. (FIG. 2E) Half-maximal inhibitory concentration (IC50) determination for the SHP2 inhibitor RMC-4550 in the indicated ROS1 patient-derived cell lines based on crystal violet quantification of the experiment in (FIG. 2D). Data represent three independent experiments. Data represented as mean +/−s.e.m.

FIG. 3 Effects of MEK activation in ROS1 fusion oncoprotein-expressing cellular models. (FIGS. 3A-3C) Representative crystal violet staining of (FIG. 3A) HCC78, (FIG. 3B) CUTO-2, and (FIG. 3C) CUTO-23 cells expressing either constitutively active MEK-DD or empty vector, treated for 6 days with the indicated doses of crizotinib (criz). Quantification of n=3 experiments is shown below the wells. (FIGS. 3D-3F) Immunoblot analysis of patient-derived cell lines (FIG. 3D) HCC78, (FIG. 3E) CUTO-2, and (FIG. 3F) CUTO-23 expressing either empty vector (EV) or MEK-DD, treated with DMSO or 1 uM crizotinib for 30 minutes. Data shown represent 3 independent experiments.

FIG. 4 JAK/STAT pathway activation is unable to rescue ROS1 fusion-positive patient-derived cells from crizotinib sensitivity. (FIGS. 4A-4C) Crystal violet staining and (FIGS. 4D-4F) quantification of the ROS1 fusion-positive patient-derived cell lines (FIG. 4A, FIG. 4D) HCC78, (FIG. 4B, FIG. 4E) CUTO-2, and (FIG. 4C, FIG. 4F) CUTO-23, expressing empty vector or constitutively active STAT3, treated with DMSO or a dose-response of the ROS1 inhibitor crizotinib (criz) for 6 days. Quantification of average of n=3 experiments is shown below the wells. (FIGS. 4G-4I) Immunoblot analysis of (FIG. 4G) HCC78, (FIG. 4H) CUTO-2, and (FIG. 4I) CUTO-23 cells expressing either empty vector (EV) or CA-STAT3, treated for 30 minutes with either DMSO (-) or 1 uM crizotinib. Crystal violet and immunoblot data represent 3 independent experiments. Data in (FIGS. 4D-4F) are shown as mean +/−s.e.m.

FIG. 5 Effects of MAPK pathway suppression by SHP2 inhibitor treatment in ROS1-fusion oncoprotein expressing patient-derived NSCLC cell lines. (FIGS. 5A-5C) Immunoblot analysis of (FIG. 5A) HCC78, (FIG. 5B) CUTO-2, and (FIG. 5C) CUTO-23 cells treated for 30 minutes with DMSO, 0.1 μM, or 1 μM of the SHP2 inhibitor (SHP2i) RMC-4550. Data represent 3 independent experiments. (FIGS. 5D-5G) Representative crystal violet staining of (FIG. 5D) HCC78, (FIG. 5E) CUTO-2, (FIG. 5F) CUTO-23, and (FIG. 5G) CUTO-33 cells treated for 6 days with the indicated doses of RMC-4550. Quantification of average of n=3 experiments is shown below the wells.

FIG. 6 ROS1 exonic breakpoint does not determine the fusion protein's ability to engage the MAPK pathway. Immunoblot of 293T cells expressing SDC4-ROS fusions harboring a ROS1 breakpoint in either exon 32 or exon 34. Both isoforms are able to activate the MAPK pathway (as measured by phospho-ERK) to the same degree when expressed at similar protein levels.

FIG. 7 Localization of ROS1 protein in isogenic BEAS-2B system reveals different localization of fusion oncoproteins. Immunofluorescence and confocal microscopy of BEAS-2B cells stably expressing SDC4-ROS1, SLC34A2-ROS1, and CD74-ROS1. Rows 1,2=SDC4-ROS1; Rows 3,4=SLC34A2-ROS1; Rows 5,6=CD74-ROS1. Antibodies used were specific for: (A-F)=ROS1; (G,I,K)=EEA1; (H,J,L)=Calnexin; and (M-R)=DAPI; (S-X)=overlay image of the left 3 columns, with (right-most column) adjacent high magnification image of representative cells (outlined by white boxes). Images are representative of ≥10 fields and at least 2 biological replicate experiments.

FIG. 8 Localization of ROS1 in patient-derived cell lines reveals differential subcellular localization of the different ROS1 fusion oncoproteins. Immunofluorescence and confocal microscopy of patient-derived cell lines expressing (A-B), SDC4-ROS1, (C-D) SLC34A2-ROS, and (E-F, G-H) CD74-ROS1. The last two columns show an overlay image of the left 3 columns, with increased magnification of individual representative cells shown in the right-most column and the cells highlighted indicated in white boxes. Images are representative of ≥10 fields and at least 2 independent experiments.

FIG. 9 Localization of ROS1 oncoproteins regulates engagement of downstream signaling pathways. (FIG. 9A) Immunofluorescence and confocal microscopy of BEAS2-B cells stably expressing an endosome-targeted FYVE-tagged CD74-ROS construct and stained with the indicated antibodies. Far right panel=increased magnification of a representative individual cell. Confocal images are representative of ≥10 fields and at least 2 independent experiments. (FIG. 9B) Immunoblot analysis of BEAS2-B cells transfected with GFP, WT CD74-ROS1, or FYVE-CD74-ROS1. Immunoblot is representative of 3 independent experiments.

FIG. 10 MAPK pathway activation in ROS1 fusion oncoprotein-driven cancer models is associated with increased tumorigenic properties in vivo. (FIG. 10A) Immunoblot analysis of ROS1 fusion oncoprotein expression in isogenic NIH-3T3 cells. (FIG. 10B) Tumor growth rates of tumor xenografts of NIH-3T3 ROS1 fusion oncoprotein-expressing cells described in (FIG. 10A) implanted into the flanks of immunocompromised mice. (FIG. 10C) Tumor growth rates of tumor xenografts of NIH-3T3 cells expressing CD74-ROS1 WT or FYVE-tagged CD74-ROS1. (FIG. 10D) Immunoblot analysis of NIH-3T3 tumor xenograft explants expressing wild-type (WT) or FYVE-tagged CD74-ROS1. Each lane represents an individual tumor. Data in (FIGS. 10B-10C) are shown as the mean of 6 tumors +/−s.e.m.

FIG. 11 Prevalence of N-terminal fusion partners and ROS1 exonic breakpoints. (FIG. 11A) Prevalence of ROS1 fusion partners present in COSMIC data set (in pie chart), and other ROS1 fusion partners identified in case reports. (FIG. 11B) Analysis of COSMIC data on ROS1 fusions demonstrate bias within fusions for specific exonic breakpoints.

FIG. 12 Effect of RMC-4550 on ERK phosphorylation in EML4-ALK fusion cell line. Treatment of NCI-H3122 Lung adenocarcinoma cells, which express an EML4-ALK fusion, with RMC-4550 results in a dose-dependent inhibition of ERK phosphorylation as measured with AlphaLISA SureFire Ultra HV pERK Assay Kit (Perkin Elmer).

FIG. 13 Effect of RMC-4550 on cell proliferation of EML4-ALK fusion cell line. Treatment of NCI-H3122 Lung adenocarcinoma cells, which express an EML4-ALK fusion, with RMC-4550 results in a dose-dependent inhibition of cell proliferation as assessed using the 3D CellTiter-Glo (CTG) kit (Promega).

FIG. 14 Effect of RMC-4550 on ERK phosphorylation in CCDC6-RET fusion cell line. Treatment of LC-2/AD Lung adenocarcinoma cells, which express an CCDC6-RET fusion, with RMC-4550 results in a dose-dependent inhibition of ERK phosphorylation as measured with AlphaLISA SureFire Ultra HV pERK Assay Kit (Perkin Elmer).

### DETAILED DESCRIPTION OF THE INVENTION

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference in their entireties.

### General Methods

The practice of the present invention will employ, unless otherwise indicated, conventional techniques of cell culturing, molecular biology (including recombinant techniques), microbiology, cell biology, biochemistry and immunology, which are within the skill of the art. Such techniques are explained fully in the literature, such as, Molecular Cloning; A Laboratory Manual, third edition (Sambrook et al., 2001) Cold Spring Harbor Press; Oligonucleotide Synthesis (P. Herdewijn, ed., 2004); Animal Cell Culture (R. I. Freshney), ed., 1987); Methods in Enzymology (Academic Press, Inc.); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR; The Polymerase Chain Reaction, (Mullis et al., eds., 1994); Current Protocols in Immunology (J. E. Coligan et al., eds., 1991); Short Protocols in Molecular Biology (Wiley and Sons, 1999); Manual of Clinical Laboratory Immunology (B. Detrick, N. R. Rose, and J. D. Folds eds., 2006); Immunochemical Protocols (J. Pound, ed., 2003); Lab Manual in Biochemistry; Immunology and Biotechnology (A. Nigam and A. Ayyagari, eds. 2007); Immunology Methods Manual; The Comprehensive Sourcebook of Techniques (Ivan Lefkovits, ed., 1996); Using Antibodies: A Laboratory Manual (E. Harlow and D. Lane, eds., 1988); and others.

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “e.g.” is used herein to mean “for example,” and will be understood to imply the inclusion of a stated step or element or group of steps or elements but not the exclusion of any other step or element or group of steps or elements.

By “optional” or “optionally,” it is meant that the subsequently described event or circumstance may or may not occur, and that the description includes instances where the event or circumstance occurs and instances in which it does not. For example, “optionally substituted aryl” encompasses both “aryl” and “substituted aryl” as defined herein. It will be understood by those ordinarily skilled in the art, with respect to any group containing one or more substituents, that such groups are not intended to introduce any substitution or substitution patterns that are sterically impractical, synthetically non-feasible, and/or inherently unstable.

The term “administer”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

The term “Sample” or “biological sample,” as used herein, refers to a sample obtained from a subject, e.g., a human subject or a patient, which may be tested for a particular molecule, for example one or more of the RTK fusions described herein (e.g., a ROS1 fusion, an ALK fusion, a RET fusion, an NTRK1 fusion, an NTRK2 fusion, or an NTRK3 fusion). Samples may include, but are not limited to, biopsies, tissues, cells, buccal swab sample, body fluids, including blood, serum, plasma, urine, saliva, cerebral spinal fluid, tears, pleural fluid and the like. In some embodiments, the samples that are suitable for use in the methods described herein contain genetic material, e.g., genomic DNA (gDNA). In some embodiments, the samples contain nucleotides, e.g., RNA (e.g., mRNA) or cDNA derived from mRNA. In some embodiments, the samples contain protein. Methods and reagents are known in the art for obtaining, processing, and analyzing samples. The sample may be further processed before the detecting step. For example, DNA or protein in a cell or tissue sample can be separated from other components of the sample. The sample can be concentrated and/or purified to isolate DNA and/or protein. Cells can be harvested from a biological sample using standard techniques known in the art. For example, cells can be harvested by centrifuging a cell sample and resuspending the pelleted cells. The cells can be resuspended in a buffered solution such as phosphate-buffered saline (PBS). After centrifuging the cell suspension to obtain a cell pellet, the cells can be lysed to extract DNA, e.g., genomic DNA, and/or protein. All samples obtained from a subject, including those subjected to any sort of further processing, are considered to be obtained from the subject.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The terms “Compound A,” “Cmp A,” “RMC-3943,” and “RMC-0693943” are used interchangeably herein to refer to a SHP2 inhibitor having the following structure:

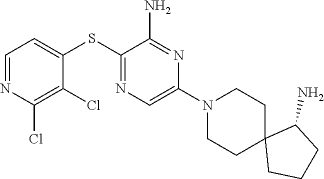 [SMILES: Nc1nc(N2CCC3(CCC[C@H]3N)CC2)cnc1Sc1ccnc(Cl)c1Cl]

The terms “Compound B,” “Cmp B,” “RMC-4550,” and “RMC-0694550” are used interchangeably herein to refer to a SHP2 inhibitor having the following structure:

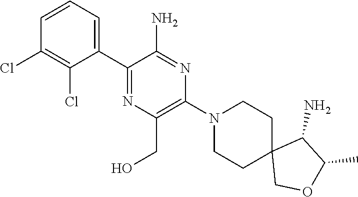 [SMILES: C[C@@H]1OCC2(CCN(c3nc(N)c(-c4cccc(Cl)c4Cl)nc3CO)CC2)[C@@H]1N]

The terms “Compound C,” and “Cmp C,” are used interchangeably herein to refer to an allosteric SHP2 inhibitor compound of similar structure to RMC-3943 and RMC-4550. Compound C is disclosed in PCT/US2017/041577 (WO 2018/013597), incorporated herein by reference in its entirety.

Example 9 shows the SHP2 inhibitory activity of each of RMC-3943, RMC-4550, and Compound C.

The term SHP099 refers to a SHP2 inhibitor having the following structure:

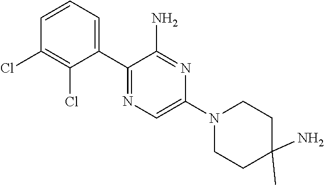 [SMILES: CC1(N)CCN(c2cnc(-c3cccc(Cl)c3Cl)c(N)n2)CC1]

The term “disorder” is used in this disclosure to mean, and is used interchangeably with, the terms disease, condition, or illness, unless otherwise indicated.

An “effective amount,” when used in connection with a compound, is an amount of the compound, e.g., a SHP2 inhibitor, needed to elicit a desired response. In some embodiments, the desired response is a biological response, e.g., in a subject. In some embodiments, the compound (e.g., a SHP2 inhibitor) may be administered to a subject in an effective amount to effect a biological response in the subject. In some embodiments, the effective amount is a “therapeutically effective amount.”

The term “inhibitor” means a compound that prevents a biomolecule, (e.g., a protein, nucleic acid) from completing or initiating a reaction. An inhibitor can inhibit a reaction by competitive, uncompetitive, or non-competitive means. Exemplary inhibitors include, but are not limited to, nucleic acids, DNA, RNA, shRNA, siRNA, proteins, protein mimetics, peptides, peptidomimetics, antibodies, small molecules, chemicals, analogs that mimic the binding site of an enzyme, receptor, or other protein, e.g., that is involved in signal transduction, therapeutic agents, pharmaceutical compositions, drugs, and combinations of these. In some embodiments, the inhibitor can be nucleic acid molecules including, but not limited to, siRNA that reduce the amount of functional protein in a cell. Accordingly, compounds said to be “capable of inhibiting” a particular protein, e.g., SHP2, comprise any such inhibitor.

The term “allosteric SHP2 inhibitor” means a small-molecule compound capable of inhibiting SHP2 through binding to SHP2 at a site other than the active site of the enzyme. Exemplary allosteric SHP2 inhibitors disclosed herein include, without limitation: (i) RMC-3943; (ii) RMC-4550; (iii) Compound C; (iv) SHP099; (v) an allosteric SHP2 inhibitor compound of any one of Formula I, of Formula II, of Formula III, of Formula I-V1, of Formula I-V2, of Formula I-W, of Formula I-X, of Formula I-Y, of Formula I-Z, of Formula IV, of Formula V, of Formula VI, of Formula IV-X, of Formula IV-Y, of Formula IV-Z, of Formula VII, of Formula VIII, of Formula IX, and of Formula X, disclosed herein; (vi) TN0155, (vii) a compound from Table 1, disclosed herein; (viii) a compound from Table 2, disclosed herein; and (ix) combinations thereof. The term “mutation” as used herein indicates any modification of a nucleic acid and/or polypeptide which results in an altered nucleic acid or polypeptide. The term “mutation” may include, for example, point mutations, deletions or insertions of single or multiple residues in a polynucleotide, which includes alterations arising within a protein-encoding region of a gene as well as alterations in regions outside of a protein-encoding sequence, such as, but not limited to, regulatory or promoter sequences, as well as amplifications and/or chromosomal breaks or translocations.

A “patient” or “subject” is a mammal, e.g., a human, mouse, rat, guinea pig, dog, cat, horse, cow, pig, or non-human primate, such as a monkey, chimpanzee, baboon or rhesus.

The term “prevent” or “preventing” with regard to a subject refers to keeping a disease or disorder from afflicting the subject. Preventing includes prophylactic treatment. For instance, preventing can include administering to the subject a compound disclosed herein before a subject is afflicted with a disease and the administration will keep the subject from being afflicted with the disease.

The term “providing to a/the subject” a therapeutic agent, e.g., a SHP2 inhibitor, includes administering such an agent.

The terms “RAS pathway” and “RAS/MAPK pathway” are used interchangeably herein to refer to a signal transduction cascade downstream of various cell surface growth factor receptors in which activation of RAS (and its various isoforms and alleotypes) is a central event that drives a variety of cellular effector events that determine the proliferation, activation, differentiation, mobilization, and other functional properties of the cell. SHP2 conveys positive signals from growth factor receptors to the RAS activation/deactivation cycle, which is modulated by guanine nucleotide exchange factors (GEFs, such as SOS1) that load GTP onto RAS to produce functionally active GTP-bound RAS as well as GTP-accelerating proteins (GAPs, such as NF1) that facilitate termination of the signals by conversion of GTP to GDP. GTP-bound RAS produced by this cycle conveys essential positive signals to a series of serine/threonine kinases including RAF and MAP kinases, from which emanate additional signals to various cellular effector functions.

The term “SHP2” means “Src Homology 2 domain-containing protein tyrosine phosphatase 2” and is also known as SH-PTP2, SH-PTP3, Syp, PTP1D, PTP2C, SAP-2 or PTPN11. Numbering of SHP2 mutations in the present disclosure is according to Uniprot Isoform 2 (accession number Q06124-2)

A “therapeutic agent” is any substance, e.g., a compound or composition, capable of treating a disease or disorder. In some embodiments, therapeutic agents that are useful in connection with the present disclosure include without limitation SHP2 inhibitors, ALK inhibitors, MEK inhibitors, RTK inhibitors (TKIs), and cancer chemotherapeutics. Many such inhibitors are known in the art and are disclosed herein.

The terms “therapeutically effective amount” and “therapeutic dose” are used interchangeably herein to refer to an amount of a compound, e.g., a SHP2 inhibitor, which is effective following administration to a subject for treating a disease or disorder in the subject as described herein.

The term “prophylactically effective amount” is used herein to refer to an amount of a compound, e.g., a SHP2 inhibitor, which is effective following administration to a subject, for preventing or delaying the onset of a disease or disorder in the subject as described herein.

The term “treatment” or “treating” with regard to a subject, refers to improving at least one symptom, pathology or marker of the subject's disease or disorder, either directly or by enhancing the effect of another treatment. Treating includes curing, improving, or at least partially ameliorating the disorder, and may include even minimal changes or improvements in one or more measurable markers of the disease or condition being treated. “Treatment” or “treating” does not necessarily indicate complete eradication or cure of the disease or condition, or associated symptoms thereof. The subject receiving this treatment is any subject in need thereof. Exemplary markers of clinical improvement will be apparent to persons skilled in the art.

### Overview

The present disclosure relates to, inter alia, methods, compositions, and kits for treating or preventing a disease or disorder (e.g., cancer) with a SHP2 inhibitor alone or in combination with another suitable therapeutic agent. Specifically, in some aspects, the present disclosure is concerned with stratifying subjects having a disease or disorder (e.g., cancer) as candidates for treatment with an allosteric SHP2 inhibitor based on the presence of certain oncogenic tyrosine kinase fusion mutations in the subject.

As described in the accompanying Examples, Applicants studied various RTK fusions to investigate the potential role that the RTK fusion partner might play in modulating the activity of the RTK and oncogenesis. Applicants have surprisingly demonstrated that distinct N-terminal fusion partners drive differential subcellular localization that imparts distinct cell signaling and oncogenic properties of different, clinically-relevant, RTK fusion oncoproteins. SDC4-ROS1 and SLC34A2-ROS1 fusion oncoproteins reside on endosomes and activate the RAS/MAPK pathway, and cells expressing these fusion oncoproteins are sensitive to treatment with SHP2 inhibitors. CD74-ROS1 instead localizes to the endoplasmic reticulum (ER), fails to activate RAS/MAPK, and cells expressing this fusion oncoprotein are insensitive to treatment with SHP2 inhibitors. Forced re-localization of CD74-ROS1 from the ER to endosomes restores RAS/MAPK signaling and ROS1 fusion oncoproteins that better activate RAS/MAPK form more aggressive tumors. Thus, differential subcellular localization, controlled by the N-terminal fusion partner, regulates the oncogenic mechanisms and output of certain RTK fusion oncoproteins.

Accordingly, the present disclosure is based, in part, on the surprising discovery that some, but not all, oncogenic tyrosine kinase fusion mutations lead to activation of the RAS/MAPK pathway, and cancers having such mutations are particularly susceptible to treatment with a SHP2 inhibitor. Moreover, the present disclosure is based, in part, on the surprising discovery that the subcellular localization of such oncogenic tyrosine kinase fusions may play a role in altering the downstream signaling of the RTKs and in oncogenesis.

Thus, in some aspects, the present disclosure provides a method for identifying whether a subject has a cancer that is sensitive to SHP2 inhibition by determining whether the cancer comprises a MAPK-activating RTK fusion. Such a determination may be utilized in patient stratification, wherein a patient having a cancer that comprises a MAPK-activating RTK fusion may be administered a SHP2 inhibitor alone or in combination with one or more additional other therapeutic agents.

As used herein, “patient stratification” means classifying one or more patient as having a disease or disorder (e.g., cancer) that is either likely or unlikely to be treatable with a therapeutic agent (e.g., an allosteric SHP2 inhibitor). The terms “patient stratification” and “subject stratification” may be used interchangeably.

Patient stratification may comprise classifying a patient as having a tumor that is sensitive to treatment with an allosteric SHP2 inhibitor. The patient stratification may be based on the presence or absence of a tumor comprising one or more cell containing an RTK fusion that is oncogenic and that activates the MAPK pathway.

The term “oncogenic RTK fusion” means an RTK fusion that is associated with cancer. In some embodiments the term encompasses fusions that are independently oncogenic (i.e., “cancer-driving” RTK fusions) and fusions that are oncogenic when they occur in combination with one or more other oncogenic mutation.

The presence of an oncogenic RTK fusion that activates the MAPK pathway may be determined, e.g., for the purpose of patient stratification, by any suitable method known in the art or described herein. For example, but not to be limited in any way, in some embodiments, a biological sample from a patient (e.g., a cell such as a tumor cell) may be genotyped for the presence or absence of an RTK fusion (e.g., an oncogenic RTK fusion that is known to activate the MAPK pathway). The cell or a population of such cells may additionally or alternatively be analyzed to determine whether such an RTK fusion, if present, brings about MAPK pathway activation in the patient's cell.

Activation of the MAPK pathway may be determined using any suitable method known in the art or described herein. For example, activation of the MAPK pathway may be determined by immunoblot; immunofluorescence; or ELISA; e.g., utilizing antibodies that are specific for phosphorylated versions of MAPK signaling molecules. See, e.g., Example 1.

Many suitable genotyping methods are known in the art, discussed below, and are suitable for use in the present invention. These may include, e.g., sequencing approaches, microarray approaches, mass spectrometry, high-throughput sequencing approaches, e.g., at a single molecule level.

For example, but not to be limited in anyway, in some aspects, a biological sample from a patient (e.g., a cell such as a tumor cell) may be genotyped using a hybridization detection method to determine whether the cell contains an oncogenic RTK fusion (e.g., an oncogenic RTK fusion that is known to activate the MAPK pathway).

Hybridization detection methods are based on the formation of specific hybrids between complementary nucleic acid sequences that serve to detect nucleic acid sequence mutation(s). Such methods include, e.g., microarray analysis and real time PCR. Hybridization methods, such as Southern analysis, Northern analysis, or in situ hybridizations, may also be used (see Current Protocols in Molecular Biology, Ausubel et al., eds., John Wiley & Sons 2003, incorporated by reference in its entirety).

Other suitable methods for genotyping a cell (e.g., a tumor cell) to determine whether it contains an RTK fusion (e.g., an oncogenic RTK fusion that is known to activate the MAPK pathway) include direct manual sequencing (Church and Gilbert, Proc. Natl. Acad. Sci. USA 81:1991-1995 (1988); Sanger et al., Proc. Natl. Acad Sci. USA 74:5463-5467 (1977); Beavis et al. U.S. Pat. No. 5,288,644, each incorporated by reference in its entirety for all purposes); automated fluorescent sequencing; single-stranded conformation polymorphism assays (SSCP); clamped denaturing gel electrophoresis (CDGE); two-dimensional gel electrophoresis (2DGE or TDGE); conformational sensitive gel electrophoresis (CSGE); denaturing gradient gel electrophoresis (DGGE) (Sheffield et al., Proc. Natl. Acad. Sci. USA 86:232-236 (1989)), mobility shift analysis (Orita et al., Proc. Natl. Acad. Sci. USA 86:2766-2770 (1989), incorporated by reference in its entirety), restriction enzyme analysis (Flavell et al., Cell 15:25 (1978); Geever et al., Proc. Natl. Acad. Sci. USA 78:5081 (1981), incorporated by reference in its entirety); quantitative real-time PCR (Raca et al., Genet Test 8(4):387-94 (2004), incorporated by reference in its entirety); heteroduplex analysis; chemical mismatch cleavage (CMC) (Cotton et al., Proc. Natl. Acad. Sci. USA 85:4397-4401 (1985), incorporated by reference in its entirety); RNase protection assays (Myers et al., Science 230:1242 (1985), incorporated by reference in its entirety); use of polypeptides that recognize nucleotide mismatches, e.g., E. coli mutS protein; allele-specific PCR, for example. See, e.g., U.S. Patent Publication No. 2004/0014095, which is incorporated herein by reference in its entirety.

In one embodiment, genomic DNA (gDNA) or a fragment (“region”) thereof containing the site of an RTK fusion present in the sample obtained from the subject, is first amplified. The RTK fusion gDNA, in one embodiment, is one or more of the oncogenic RTK fusions described herein. Such regions can be amplified and isolated by PCR using oligonucleotide primers designed based on genomic and/or cDNA sequences that flank the site. See e.g., PCR Primer: A Laboratory Manual, Dieffenbach and Dveksler, (Eds.); McPherson et al., PCR Basics: From Background to Bench (Springer Verlag, 2000, incorporated by reference in its entirety); Mattila et al., Nucleic Acids Res., 19:4967 (1991), incorporated by reference in its entirety; Eckert et al., PCR Methods and Applications, 1:17 (1991), incorporated by reference in its entirety; PCR (eds. McPherson et al., IRL Press, Oxford), incorporated by reference in its entirety; and U.S. Pat. No. 4,683,202, incorporated by reference in its entirety. Other amplification methods that may be employed include the ligase chain reaction (LCR) (Wu and Wallace, Genomics, 4:560 (1989), Landegren et al., Science, 241:1077 (1988), transcription amplification (Kwoh et al., Proc. Natl. Acad. Sci. USA, 86:1173 (1989)), self-sustained sequence replication (Guatelli et al., Proc. Nat. Acad. Sci. USA, 87:1874 (1990)), incorporated by reference in its entirety, and nucleic acid based sequence amplification (NASBA). Guidelines for selecting primers for PCR amplification are known to those of ordinary skill in the art. See, e.g., McPherson et al., PCR Basics: From Background to Bench, Springer-Verlag, 2000, incorporated by reference in its entirety. A variety of computer programs for designing primers are available.

In one example, a sample (e.g., a sample comprising genomic DNA), is obtained from a subject. The DNA in the sample is then examined to determine its RTK fusion profile and as described herein. The term “RTK fusion profile” refers to presence or absence of any one or more known RTK fusion mutations (including, e.g., an oncogenic RTK fusion described herein). The profile is determined by any method described herein, e.g., by sequencing or by hybridization of the gene in the genomic DNA, RNA, or cDNA to a nucleic acid probe, e.g., a DNA probe (which includes cDNA and oligonucleotide probes) or an RNA probe. The nucleic acid probe can be designed to specifically or preferentially hybridize with a gDNA region on the RTK fusion.

In some embodiments, restriction digest analysis can be used to detect the existence of an RTK fusion, if alternate RTK fusion result in the creation or elimination of a restriction site. A sample containing genomic DNA is obtained from the individual. Polymerase chain reaction (PCR) can be used to amplify a region comprising the RTK fusion site (e.g., the C-terminus of the protein fused to the RTK and the N-terminus of the RTK protein), and restriction fragment length analysis s conducted (see Current Protocols in Molecular Biology, Ausubel et al., eds., John Wiley & Sons 2003, incorporated by reference in its entirety). The digestion pattern of the relevant DNA fragment indicates the presence or absence of a particular RTK fusion and is therefore indicative of the presence or absence of susceptibility to treatment with a SHP2 inhibitor.

Sequence analysis can also be used to detect the one or more RTK fusions (e.g., an oncogenic RTK fusion described herein). A sample comprising DNA or RNA is obtained from the subject. PCR or other appropriate methods can be used to amplify a portion encompassing the RTK fusion site, if desired. The sequence is then ascertained, using any standard method, and the presence of an RTK fusion is determined.

Allele-specific oligonucleotides can also be used to detect the presence of an RTK fusion, e.g., through the use of dot-blot hybridization of amplified oligonucleotides with allele-specific oligonucleotide (ASO) probes (see, for example, Saiki et al., Nature (London) 324:163-166 (1986)). An “allele-specific oligonucleotide” (also referred to herein as an “allele-specific oligonucleotide probe”) is typically an oligonucleotide of approximately 10-50 base pairs, preferably approximately 15-30 base pairs, that specifically hybridizes to a nucleic acid region that contains an RTK fusion. An allele-specific oligonucleotide probe that is specific for a particular RTK fusion can be prepared using standard methods (see Current Protocols in Molecular Biology, Ausubel et al., eds., John Wiley & Sons 2003, incorporated by reference in its entirety).

In some embodiments, to determine which of RTK fusions are present in a subject, a sample comprising DNA may be obtained from the subject. PCR or another amplification procedure may be used to amplify a portion encompassing the RTK fusion site.

Real-time pyrophosphate DNA sequencing is yet another approach to detection of RTK fusion s (Alderborn et al., (2000) Genome Research, 10(8): 1249-1258, incorporated by reference in its entirety). Additional methods include, for example, PCR amplification in combination with denaturing high performance liquid chromatography (dHPLC) (Underhill et al., Genome Research, Vol. 7, No. 10, pp. 996-1005, 1997, incorporated by reference in its entirety for all purposes).

High throughput sequencing, or next-generation sequencing can also be employed to detect one or more of the RTK fusions described herein. Such methods are known in the art (see e.g., Zhang et al., J Genet Genomics. 2011 Mar. 20; 38(3):95-109, incorporated by reference in its entirety for all purposes; Metzker, Nat Rev Genet. 2010 January; 11(1):31-46, incorporated by reference in its entirety for all purposes) and include, but are not limited to, technologies such as ABI SOLiD sequencing technology (now owned by Life Technologies, Carlsbad, Calif.); Roche 454 FLX which uses sequencing by synthesis technology known as pyrosequencing (Roche, Basel Switzerland); Illumina Genome Analyzer (Illumina, San Diego, Calif.); Dover Systems Polonator G.007 (Salem, N.H.); Helicos (Helicos BioSciences Corporation, Cambridge Mass., USA), and Sanger. In one embodiment, DNA sequencing may be performed using methods well known in the art including mass spectrometry technology and whole genome sequencing technologies, single molecule sequencing, etc.

In one embodiment, nucleic acid, for example, genomic DNA is sequenced using nanopore sequencing, to determine the presence of the one or more RTK fusions described herein (e.g., as described in Soni et al. (2007). Clin Chem 53, pp. 1996-2001, incorporated by reference in its entirety for all purposes). Nanopore sequencing is a single-molecule sequencing technology whereby a single molecule of DNA is sequenced directly as it passes through a nanopore. A nanopore is a small hole, of the order of 1 nanometer in diameter. Immersion of a nanopore in a conducting fluid and application of a potential (voltage) across it results in a slight electrical current due to conduction of ions through the nanopore. The amount of current which flows is sensitive to the size and shape of the nanopore. As a DNA molecule passes through a nanopore, each nucleotide on the DNA molecule obstructs the nanopore to a different degree, changing the magnitude of the current through the nanopore in different degrees. Thus, this change in the current as the DNA molecule passes through the nanopore represents a reading of the DNA sequence. Nanopore sequencing technology as disclosed in U.S. Pat. Nos. 5,795,782, 6,015,714, 6,627,067, 7,238,485 and 7,258,838 and U.S. Patent Application Publication Nos. 2006/003171 and 2009/0029477, each incorporated by reference in its entirety for all purposes, is amenable for use with the methods described herein.

In some embodiments, the present disclosure provides a method for using a SHP2 inhibitor to treat a subject with a cancer, the method comprising the steps of: (i) determining whether the cancer comprises a cell that contains an oncogenic tyrosine kinase fusion that causes MAPK activation; and (ii) administering the SHP2 inhibitor to the patient if the cancer comprises a cell that contains an oncogenic tyrosine kinase fusion that causes MAPK activation. Such methods may further comprise administering one or more other therapeutic agent (e.g., an inhibitor of a MAP kinase pathway or an anti-cancer therapeutic agent) to the subject in combination with the SHP2 inhibitor (e.g., as a combination therapy). Such methods may additionally or alternatively further comprise administering an additional therapy, e.g., an additional cancer therapy. For example, in some embodiments, the SHP2 inhibitor is administered according to the above method in combination with a cancer therapy selected from a chemotherapy, immunotherapy, radiation therapy, and surgical tumor resection. In some embodiments, the SHP2 inhibitor is administered according to the above method in combination with a cancer therapy selected from a chemotherapy, immunotherapy, radiation therapy, and/or surgical tumor resection. In some embodiments, the present disclosure provides a method comprising determining whether the cancer comprises a cell that contains an oncogenic tyrosine kinase fusion that causes MAPK activation; and administering a cancer therapy selected from chemotherapy, radiation therapy, and/or surgical tumor resection, but not administering a SHP2 inhibitor, if the cancer does not comprise a cell containing an oncogenic tyrosine kinase fusion that causes MAPK activation.

In some embodiments, the present disclosure provides a method for killing cancer cells with a SHP2 inhibitor, the method comprising the steps of: (i) determining whether the cancer cells contain an oncogenic tyrosine kinase fusion that causes MAPK activation; and (ii) contacting the cancer cells with the SHP2 inhibitor if the cancer cells contains an oncogenic tyrosine kinase fusion that causes MAPK activation. In some embodiments, the contacting occurs in vivo in a subject. In some embodiments, the in vivo contacting in the subject occurs via administration of the SHP2 inhibitor to the subject. Such methods of killing cancer cells with a SHP2 inhibitor may further comprise contacting the cancer cells with the SHP2 inhibitor in combination with (e.g., as a combination therapy) one or more other therapeutic agent (e.g., an inhibitor of a MAP kinase pathway or an anti-cancer therapeutic agent). Such methods may additionally or alternatively further comprise administering an additional therapy, e.g., an additional cancer therapy. For example, in some embodiments, the SHP2 inhibitor is administered according to the above method in combination with a cancer therapy selected from a chemotherapy, immunotherapy, radiation therapy, and surgical tumor resection. In some embodiments, the SHP2 inhibitor is administered according to the above method in combination with a cancer therapy selected from a chemotherapy, immunotherapy, radiation therapy, and/or surgical tumor resection. In some embodiments, the method for killing cancer cells with a SHP2 inhibitor comprises (iii) contacting the cancer cells with a cancer therapy selected from chemotherapy, radiation therapy, and/or surgical tumor resection, but not a SHP2 inhibitor, if the cancer cells do not contain an oncogenic tyrosine kinase fusion that causes MAPK activation.

In some embodiments, the present disclosure provides a method for treating a patient with a SHP2 inhibitor, wherein the patient has cancer, the method comprising the steps of: (i) determining whether the patient has a SHP2-sensitive cancer by: (a) obtaining or having obtained a biological sample from the patient; and (b) performing or having performed an assay on the biological sample to determine if the patient has a tumor comprising a cell that contains an oncogenic tyrosine kinase fusion that causes MAPK activation; and (ii) administering the SHP2 inhibitor to the patient if the patient has a tumor comprising a cell containing an oncogenic tyrosine kinase fusion that causes MAPK activation. As shown in the Examples, in some embodiments, the presence of an oncogenic tyrosine kinase fusion that cause MAPK activation is indicative of a SHP2-sensitive cancer. Such a method may further comprise administering one or more other therapeutic agent (e.g., an inhibitor of a MAP kinase pathway or an anti-cancer therapeutic agent) to the subject in combination with the SHP2 inhibitor (e.g., as a combination therapy). Such methods may additionally or alternatively comprise administering an additional therapy (e.g., an additional cancer therapy). For example, in some embodiments, the SHP2 inhibitor is administered in combination with a cancer therapy selected from a chemotherapy, immunotherapy, radiation therapy, and surgical tumor resection to a patient identified, according to the above method, as having a SHP2 sensitive cancer. In some embodiments, the SHP2 inhibitor is administered in combination with a cancer therapy selected from a chemotherapy, immunotherapy, radiation therapy, and/or surgical tumor resection to a patient identified, according to the above method, as having a SHP2 sensitive cancer. In some embodiments, the disclosure provides a method comprising the steps of: (i) determining whether the patient has a SHP2-sensitive cancer according to the above method; and (ii) administering to the patient a cancer therapy selected from chemotherapy, radiation therapy, and/or surgical tumor resection, but not administering to the patient a SHP2 inhibitor, if the patient does not have a tumor comprising a SHP2-sensitive cancer (e.g., if the patient does not have a tumor comprising a cell containing an oncogenic tyrosine kinase fusion that causes MAPK activation).

In some embodiments, the present disclosure provides a method for treating a subject having a tumor with a SHP2 inhibitor, the method comprising: determining whether a biological sample obtained from the subject contains an oncogenic tyrosine kinase fusion protein comprising a N-terminal fusion partner that causes the fusion protein to localize in an endosome; and administering to the subject an inhibitor of SHP2 if the biological sample contains an oncogenic tyrosine kinase fusion protein comprising a N-terminal fusion partner that causes the fusion protein to localize in an endosome. Such a method may further comprise administering one or more other therapeutic agent (e.g., an inhibitor of a MAP kinase pathway or an anti-cancer therapeutic agent) to the subject in combination with the SHP2 inhibitor (e.g., as a combination therapy). Such methods may additionally or alternatively comprise administering an additional therapy (e.g., an additional cancer therapy). For example, in some embodiments, the SHP2 inhibitor is administered in combination with a cancer therapy selected from a chemotherapy, immunotherapy, radiation therapy, and surgical tumor resection to a patient identified, according to the above method, as having a SHP2 sensitive cancer. In some embodiments, the SHP2 inhibitor is administered in combination with a cancer therapy selected from a chemotherapy, immunotherapy, radiation therapy, and/or surgical tumor resection to a patient identified, according to the above method, as having a SHP2 sensitive cancer.

In certain embodiments, any one of the SHP2 inhibitors described herein for administering to a patient according to the methods disclosed herein may be administered in combination with one or more other therapeutic agent as a combination therapy. For example, a SHP2 inhibitor may be administered to a patient as a combination therapy with another agent for the treatment of a cancer comprising a cell containing an oncogenic tyrosine kinase fusion. The combination therapy may comprise administration of a SHP2 inhibitor and any other anti-cancer therapeutic agent known in the art or disclosed herein. For example, the SHP2 inhibitor may be administered to the subject in combination with an anti-cancer agent selected from, e.g., but not limited to, mitotic inhibitors such as a taxane, a vinca alkaloid, paclitaxel, docetaxel, vincristine, vinblastine, vinorelbine or vinflunine, and other anticancer agents, e.g. cisplatin, 5-fluorouracil or 5-fluoro-2-4(1H,3H)-pyrimidinedione (5FU), flutamide, gemcitabine, a checkpoint inhibitor (e.g., a checkpoint inhibitor antibody) such as, e.g., a PD-1 antibody, such as, e.g., pembrolizumab (or “Keytruda®”, Merck) nivolumab (or “Opdivo®”, BMS), PDR001 (NVS), REGN2810 (Sanofi/Regeneron), a PD-L1 antibody such as, e.g., avelumab (or “MSB0010718C” or “Bavencio®”, PFE & Merck Kga), durvalumab (or “Imfinzi®” or “MEDI-4736”, Medimmune & Celgene), atezolizumab (or “Tecentriq®” or “MPDL-3280A”, Genentech & Roche), pidilizumab (or “CT-001”, Medivation—Now Pfizer), JNJ-63723283 (JNJ), BGB-A317 (BeiGene & Celgene) or a checkpoint inhibitor disclosed in Preusser, M. et al. (2015) Nat. Rev. Neurol, (incorporated herein by reference in its entirety), including, without limitation, ipilimumab, tremelimumab, nivolumab, pembrolizumab, pidilizumab, AMP224, AMP514/MEDI0680, BMS936559, MED14736, MPDL3280A, MSB0010718C, BMS986016, IMP321, lirilumab, IPH2101, 1-7F9, and KW-6002; an RTK inhibitor, an EGFR inhibitor, an ALK inhibitor, a PI3K/AKT pathway inhibitor, an inhibitor of a MAP kinase pathway, and a MEK inhibitor. The RTK inhibitor (TKI) may inhibit, e.g., one or more RTK selected from epidermal growth factor receptor (EGFR), platelet derived growth factor receptor (PDGFR), erbB2, erbB4, vascular endothelial growth factor receptor (VEGFR), tyrosine kinase with immunoglobulin-like and epidermal growth factor homology domains (TIE-2), insulin growth factor-I (IGFI) receptor, macrophage colony stimulating factor (cfims), BTK, ckit, cmet, fibroblast growth factor (FGF) receptors, Trk receptors (TrkA, TrkB, and TrkC), ephrin (eph) receptors, hepatocyte growth factor receptors (HGFR), the RET protooncogene, and ALK. The TKI may include, but is not limited to, one or more TKI described in Cancers (Basel). 2015 September; 7(3): 1758-1784, incorporated herein by reference in its entirety. The TKI may include, but is not limited to, an EGFR inhibitor or an ALK inhibitor. The TKI may include, but is not limited to trastuzumab (Herceptin®); cetuximab (Erbitux®); panitumumab (Vectibix®); gefitinib (Iressa®); erlotinib (Tarceva®); lapatinib (Tykerb®); afatinib; sorafenib (Nexavar®); sunitinib (Sutent®); bevacizumab (Avastin®); pazopanib; nilotinib; brivanib (BMS-540215); CHIR-258 (TKI-258); SGX523; and imatinib (Gleevec®). Other TKIs that may be used according to the present disclosure in combination with a SHP2 inhibitor may include, but are not limited to, the growth factor receptor inhibitor agents described in Kath, John C., Exp. Opin. Ther. Patents (2000) 10(6):803-818; Shawver et al DDT Vol 2, No. 2 Feb. 1997; and Lofts, F. J. et al, “Growth factor receptors as targets”, New Molecular Targets for Cancer Chemotherapy, ed. Workman, Paul and Kerr, David, CRC press 1994, London, incorporated herein by reference in its entirety. The combination therapy may comprise a SHP2 inhibitor in combination with an inhibitor of the PI3K/AKT pathway (“PI3K/AKT inhibitor”) known in the art or disclosed herein. The PI3K/AKT inhibitor may include, but is not limited to, one or more PI3K/AKT inhibitor described in Cancers (Basel). 2015 September; 7(3): 1758-1784, incorporated herein by reference in its entirety. For example, the PI3K/AKT inhibitor may be selected from one or more of NVP-BEZ235; BGT226; XL765/SAR245409; SF1126; GDC-0980; PI-103; PF-04691502; PKI-587; GSK2126458. The ALK inhibitor may include, but is not limited to, ceritinib, TAE-684 (also referred to herein as “NVP-TAE694”), PF02341066 (also referred to herein as “crizotinib” or “1066”), alectinib; brigatinib; entrectinib; ensartinib (X-396); lorlatinib; ASP3026; CEP-37440; 4SC-203; TL-398; PLB1003; TSR-011; CT-707; TPX-0005, and AP26113. Additional examples of ALK kinase inhibitors are described in examples 3-39 of WO 2005016894, incorporated herein by reference in its entirety. The SHP2 inhibitor may be administered before, after, or concurrently with one or more of such anti-cancer agents. In some embodiments, such combinations may offer significant advantages, including additive or synergistic activity in therapy.

In some particular embodiments, the present disclosure provides for method for treating a disease or disorder, e.g., a cancer, with a combination therapy comprising a SHP2 inhibitor known in the art or disclosed herein in combination with an inhibitor of the MAP kinase (MAPK) pathway (or “MAPK inhibitor”) known in the art or disclosed herein. The MAPK inhibitor may be a MEK inhibitor. MAPK inhibitors for use in the methods disclosed herein may include, but are not limited to, one or more MAPK inhibitor described in Cancers (Basel). 2015 September; 7(3): 1758-1784, incorporated herein by reference in its entirety. For example, the MAPK inhibitor may be selected from one or more of Trametinib, Binimetinib, Selumetinib, Cobimetinib, LErafAON (NeoPharm), ISIS 5132; Vemurafenib, Pimasertib, TAK733, RO4987655 (CH4987655); CI-1040; PD-0325901; CH5126766; MAP855; AZD6244; Refametinib (RDEA 119/BAY 86-9766); GDC-0973/XL581; AZD8330 (ARRY-424704/ARRY-704); RO5126766 (Roche, described in PLoS One. 2014 Nov. 25; 9(11), incorporated herein by reference in its entirety); and GSK1120212 (or “JTP-74057”, described in Clin Cancer Res. 2011 Mar. 1; 17(5):989-1000, incorporated herein by reference in its entirety). The SHP2 inhibitor may be administered before, after, or concurrently with one or more of such MAPK inhibitors. In some embodiments, such combinations may offer significant advantages, including additive or synergistic activity in therapy.

In some embodiments, the present disclosure provides for method for treating a disease or disorder, e.g., a cancer, with a combination therapy comprising a SHP2 inhibitor in combination with an inhibitor of RAS, such as AMG 510, BI-2852, or ARS-3248.

In some particular embodiments, the present disclosure provides for method for treating a disease or disorder, e.g., a cancer, with a combination therapy comprising a SHP2 inhibitor known in the art or disclosed herein in combination with an inhibitor of the MAP kinase (MAPK) pathway (or “MAPK inhibitor”) known in the art or disclosed herein, and in combination with any one or more anti-cancer agent disclosed above. The SHP2 inhibitor may be administered before, after, or concurrently with one or more of such MAPK inhibitors. In some embodiments, such combinations may offer significant advantages, including additive or synergistic activity in therapy.

Any disease or condition associated with an oncogenic RTK fusion that activates MAPK may be identified, assessed, and/or treated according to the present disclosure. In particular embodiments, the oncogenic RTK fusion that activates MAPK leaves the mutated cell sensitive to allosteric inhibitors of SHP2. Several such diseases or conditions that may be treatable according to the instant disclosure are known in the art. For example, in certain embodiments, the present disclosure provides methods for treating a disease or condition selected from, but not limited to, tumors of hemopoietic and lymphoid system including myeloproliferative syndromes, myelodysplastic syndromes, and leukemia, e.g., acute myeloid leukemia, and juvenile myelomonocytic leukemias; esophageal cancer; breast cancer; lung cancer; colon cancer; gastric cancer, neuroblastoma, bladder cancer, prostate cancer; glioblastoma; urothelial carcinoma, uterine carcinoma, adenoid and ovarian serous cystadenocarcinoma, paraganglioma, phaeochromocytoma, pancreatic cancer, adrenocortical carcinoma, stomach adenocarcinoma, sarcoma, rhabdomyosarcoma, lymphoma, head and neck cancer, skin cancer, peritoneum cancer, intestinal cancer (small and large intestine), thyroid cancer, endometrial cancer, cancer of the biliary tract, soft tissue cancer, ovarian cancer, central nervous system cancer (e.g., primary CNS lymphoma), stomach cancer, pituitary cancer, genital tract cancer, urinary tract cancer, salivary gland cancer, cervical cancer, liver cancer, eye cancer, cancer of the adrenal gland, cancer of autonomic ganglia, cancer of the upper aerodigestive tract, bone cancer, testicular cancer, pleura cancer, kidney cancer, penis cancer, parathyroid cancer, cancer of the meninges, vulvar cancer and melanoma comprising a method disclosed herein, such as, e.g., a monotherapy or combination therapy disclosed herein comprising a SHP2 inhibitor.

In some instances, administration of a SHP2 inhibitor to a patient having a cancer that comprises a MAPK-activating RTK fusion may result in improvements in efficacy that are more than additive over administration of the SHP2 inhibitor to the general population of patients with the cancer. For example, in certain aspects, the present disclosure provides for patient stratification for treatment with a SHP2 inhibitor based on the presence or absence of a MAPK-activating RTK fusion in a cancer cell of a subject, wherein administering a SHP2 inhibitor to the patient that has been determined to have a such a MAPK-activating RTK fusion results in a synergistic treatment of the cancer as compared to the treatment that would be expected to result from administration of the SHP2 inhibitor to the general population of patients with the cancer. The effectiveness of the treatment may be based on any detectable readout. For example, in some instances, the synergistic treatment is based on reductions in tumor burden. In some instances, the synergistic treatment is based on SHP2-inhibitor induced tumor killing.

The RTK fusion may be an oncogenic RTK fusion. Numerous RTK fusions are known to play a role in oncogenesis. For example, the RTK fusion may in some instances be selected from an ALK fusion, a ROS1, fusion, a RET fusion, and an NTRK fusion (e.g., NTRK1). The NTRK fusion may additionally or alternatively be an NTRK2 or an NTRK3 fusion. The RTK fusion may comprise the RTK and at least a portion of SDC4, SLC34A2, FIG, LRIG3, EZR, TPM3, CD74, GOPC, KDELR3, CCDC6, or EML4. For example, the RTK fusion may comprise a protein selected SDC4, SLC34A2, FIG, LRIG3, EZR, TPM3, CD74, GOPC, KDELR3, CCDC6, or EML4 fused to a ALK, ROS1, RET, NTRK1. The RTK fusion may comprise a protein selected SDC4, SLC34A2, FIG, LRIG3, EZR, TPM3, or EML4 fused to the N-terminus of ALK, ROS1, RET, NTRK1. For example, in some aspects the RTK fusion may be selected from SDC4-ROS1, SLC34A2-ROS1, FIG-ROS1, LRIG3-ROS1, EZR-ROS1, TPM3-ROS1, CD74-ROS1, GOPC-ROS1, KDELR3v, CCDC6-ROS1. In particular aspects, the RTK fusion may be selected from an SDC4-ROS1 fusion; and SLC34A2-ROS1 fusion. In particular aspects the RTK fusion may be selected from a FIG-ROS1 fusion; a LRIG3-ROS1 fusion; an EZR-ROS1 fusion, and a TPM3-ROS1 fusion. In particular aspects the RTK fusion may be an EML4-ALK fusion. In some aspects, the RTK fusion may be selected from an ETV6-NTRK3 fusion; a TPM3-NTRK1 fusion, a MPRIP-NTRK1 fusion, a CD74-NTRK1 fusion. In some aspects, the RTK fusion may comprise a protein selected from MPRIP; CD74; RABGAP1L; TPM3; TPR; TFG; PPL; CHTOP; ARHGEF2; NFASC; BCAN; LMNA; TP53; QKI; NACC2; VCL; AGBL4; TRIM24; AFAP1; SQSTM1; ETV6; BTB1; LYN; RBPMS fused to an RTK (e.g., to an NTRK). For example, in some aspects the RTK fusion may be selected from MPRIP-NTRK1; CD74-NTRK1; RABGAP1L-NTRK1; TPM3-NTRK1; TPR-NTRK1; TFG-NTRK1; PPL-NTRK1; CHTOP-NTRK1; ARHGEF2-NTRK1; NFASC-NTRK1; BCAN-NTRK1; LMNA-NTRK1; TP53-NTRK1; QKI-NTRK2; NACC2-NTRK2; VCL-NTRK2; AGBL4-NTRK2; TRIM24-NTRK2; AFAP1-NTRK2; SQSTM1-NTRK2; ETV6-NTRK3; BTB1-NTRK3; LYN-NTRK3; RBPMS-NTRK3. In various aspects, one or more of the above-listed fusions activates the MAPK pathway.

In various embodiments, the compositions and methods disclosed herein, e.g., the methods for treating such diseases or disorders discussed herein (e.g., cancer), involve administering to a subject an effective amount of a SHP2 inhibitor or a composition (e.g., a pharmaceutical composition) comprising a SHP2 inhibitor. The terms “SHP2 inhibitor” and an “inhibitor of SHP2” are used interchangeably herein to refer to any compound or substance that is capable of inhibiting SHP2. These terms include, without limitation “allosteric SHP2 inhibitors” described herein, as well as other SHP2 inhibitors. Any such compound or substance capable of inhibiting SHP2 may be utilized in application with the present disclosure to inhibit SHP2. Non-limiting examples of SHP2 inhibitors are known in the art and are disclosed herein. For example, but not to be limited in any way, in some embodiments, the compositions and methods described herein may utilize the SHP2 inhibitor Compound C. In some embodiments, the compositions and methods described herein may utilize one or more SHP2 inhibitor selected from, but not limited to, any SHP2 inhibitor provided on Table 1 herein. In some embodiments, the compositions and methods described herein may utilize one or more SHP2 inhibitor selected from, but not limited to, any SHP2 inhibitor provided on Table 2 herein. In some embodiments, the compositions and methods described herein may utilize one or more SHP2 inhibitor selected from, but not limited to, any SHP2 inhibitor disclosed in Chen, Ying-Nan P et al., 148 Nature Vol 535 7 Jul. 2016, incorporated herein by reference in its entirety, including SHP099, disclosed therein. The compositions and methods described herein may utilize one or more SHP2 inhibitor selected from, but not limited to any SHP2 inhibitor disclosed in any one of PCT applications PCT/US2017/041577 (WO2018013597); PCT/US2018/013018 (WO 2018136264); and PCT/US2018/013023 (WO 2018136265), each of which is incorporated herein by reference in its entirety. The compositions and methods described herein may utilize one or more SHP2 inhibitor selected from, but not limited to any SHP2 inhibitor disclosed in PCT applications PCT/IB2015/050343 (WO2015107493); PCT/IB2015/050344 (WO2015107494); PCT/IB2015/050345 (WO201507495); PCT/IB2016/053548 (WO2016/203404); PCT/IB2016/053549 (WO2016203405); PCT/IB2016/053550 (WO2016203406); PCT/US2010/045817 (WO2011022440); PCT/US2017/021784 (WO2017156397); PCT/US2016/060787 (WO2017079723); and PCT/CN2017/087471 (WO 2017211303), each of which is incorporated herein by reference in its entirety. The compositions and methods described herein may utilize one or more SHP2 inhibitor selected from, but not limited to any SHP2 inhibitor disclosed in Chen L, et al., Mol Pharmacol. 2006 August; 70(2):562-70, incorporated herein by reference in its entirety, including NSC-87877 disclosed therein. The compositions and methods described herein may utilize TN0155, described under ClinicalTrials.gov Identifier: NCT03114319, available at world wide web address: clinicaltrials.gov/ct2/show/NCT03114319, incorporated herein by reference in its entirety. The compositions and methods described herein may utilize one or more SHP2 inhibitor selected from, but not limited to a SHP2 inhibitor compound of any one of Formula I, Formula II, Formula III, Formula I-V1, Formula I-V2, Formula I-W, Formula I-X, Formula I-Y, Formula I-Z, Formula IV, Formula V, Formula VI, Formula IV-X, Formula IV-Y, Formula IV-Z, Formula VII, Formula VIII, Formula IX, and Formula X, disclosed herein. In some embodiments, the compositions and methods described herein may utilize the SHP2 inhibitor Compound A. In some embodiments, the compositions and methods described herein may utilize the SHP2 inhibitor Compound RMC-4550.

Thus, in some embodiments, the compositions and methods described herein are selected from one or more SHP2 inhibitor selected from, but not limited to (i) RMC-3943, disclosed herein; (ii) RMC-4550, disclosed herein; (iii) Compound C, disclosed herein, (iv) a SHP2 inhibitor compound of any one of Formula I, Formula II, Formula III, Formula I-V1, Formula I-V2, Formula I-W, Formula I-X, Formula I-Y, Formula I-Z, Formula IV, Formula V, Formula VI, Formula IV-X, Formula IV-Y, Formula IV-Z, Formula VII, Formula VIII, Formula IX, and Formula X, disclosed herein; (v) a SHP2 inhibitor shown in Table 1, herein; (vi) a SHP2 inhibitor shown in Table 2, herein; (vii), or a combination thereof.

One aspect of the disclosure relates to compounds of Formula I:

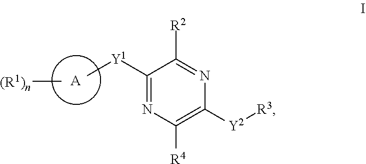

and pharmaceutically acceptable salts, prodrugs, solvates, hydrates, tautomers, or isomers thereof, wherein:

A is a 5- to 12-membered monocyclic or polycyclic cycloalkyl, heterocycloalkyl, aryl, or heteroaryl;

Y¹ is —S— or a direct bond;

Y² is —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, or —OC(O)O—; wherein the bond on the left side of Y², as drawn, is bound to the pyrazine ring and the bond on the right side of the Y² moiety is bound to R³;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R² is independently —OR^{b}, —CN, —C₁-C₆alkyl, —C₂-C₆alkenyl, —C₄-C₈cycloalkenyl, —C₂-C₆alkynyl, —C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

R^{a} is independently, at each occurrence, —H, -D, —OH, —C₃-C₈cycloalkyl, or —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;

R^{b} is independently, at each occurrence, —H, -D, —C₁-C₆alkyl, —C₃-C₈cycloalkyl, —C₂-C₆alkenyl, or heterocyclyl containing 1-5 heteroatoms selected from the group consisting of N, S, P, and O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R³ is independently —C₁-C₆alkyl or a 3- to 12-membered monocyclic or polycyclic heterocycle, wherein each alkyl or heterocycle is optionally substituted with one or more —C₁-C₆alkyl, —OH, or —NH₂; or

R³ can combine with R^{a} to form a 3- to 12-membered monocyclic or polycyclic heterocycle or a 5- to 12-membered spiroheterocycle, wherein each heterocycle or spiroheterocycle is optionally substituted with one or more —C₁-C₆alkyl, —OH, or —NH₂;

R⁴ is independently —H, -D, or —C₁-C₆alkyl, wherein each alkyl is optionally substituted with one or more —OH, —NH₂, halogen, or oxo; or

R^{a} and R⁴, together with the atom or atoms to which they are attached, can combine to form a monocyclic or polycyclic C₃-C₁₂cycloalkyl or a monocyclic or polycyclic 3- to 12-membered heterocycle, wherein the cycloalkyl or heterocycle is optionally substituted with oxo;

R⁵ and R⁶ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, or —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently, at each occurrence, 1, 2, 3, 4, 5 or 6; and

n is independently, at each occurrence, 0, 1, 2, 3, 4, 5, 6, 7, 8, 9, or 10.

Another aspect of the disclosure relates to compounds of Formula II:

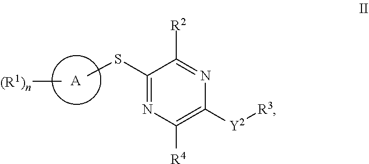

and pharmaceutically acceptable salts, prodrugs, solvates, hydrates, tautomers, or isomers thereof, wherein:

A is a 5- to 12-membered monocyclic or polycyclic cycloalkyl, heterocycloalkyl, aryl, or heteroaryl;

Y² is —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, or —OC(O)O—; wherein the bond on the left side of Y², as drawn, is bound to the pyrazine ring and the bond on the right side of the Y² moiety is bound to R³;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R² is independently —OR^{b}, —CN, —C₁-C₆alkyl, —C₂-C₆alkenyl, —C₄-C₈cycloalkenyl, —C₂-C₆alkynyl, —C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

R^{a} is independently, at each occurrence, —H, -D, —OH, —C₃-C₈cycloalkyl, or —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;

R^{b} is independently, at each occurrence, —H, -D, —C₁-C₆alkyl, —C₃-C₈cycloalkyl, —C₂-C₆alkenyl, or heterocyclyl containing 1-5 heteroatoms selected from the group consisting of N, S, P, and O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R³ is independently —C₁-C₆alkyl or a 3- to 12-membered monocyclic or polycyclic heterocycle, wherein each alkyl or heterocycle is optionally substituted with one or more —C₁-C₆alkyl, —OH, or —NH₂; or

R³ can combine with R^{a} to form a 3- to 12-membered monocyclic or polycyclic heterocycle or a 5- to 12-membered spiroheterocycle, wherein each heterocycle or spiroheterocycle is optionally substituted with one or more —C₁-C₆alkyl, —OH, or —NH₂;

R⁴ is independently —H, -D, or —C₁-C₆alkyl, wherein each alkyl is optionally substituted with one or more —OH, —NH₂, halogen, or oxo; or

R^{a} and R⁴, together with the atom or atoms to which they are attached, can combine to form a monocyclic or polycyclic C₃-C₁₂cycloalkyl or a monocyclic or polycyclic 3- to 12-membered heterocycle, wherein the cycloalkyl or heterocycle is optionally substituted with oxo;

R⁵ and R⁶ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, or —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently, at each occurrence, 1, 2, 3, 4, 5 or 6; and

n is independently, at each occurrence, 0, 1, 2, 3, 4, 5, 6, 7, 8, 9, or 10.

Another aspect of the disclosure relates to compounds of Formula III:

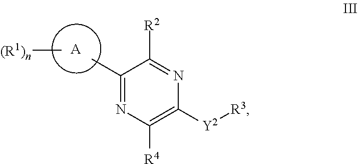

and pharmaceutically acceptable salts, prodrugs, solvates, hydrates, tautomers, or isomers thereof, wherein:

A is a 5- to 12-membered monocyclic or polycyclic cycloalkyl, heterocycloalkyl, aryl, or heteroaryl;

Y² is —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, or —OC(O)O—; wherein the bond on the left side of Y², as drawn, is bound to the pyrazine ring and the bond on the right side of the Y² moiety is bound to R³;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R² is independently —OR^{b}, —CN, —C₁-C₆alkyl, —C₂-C₆alkenyl, —C₄-C₈cycloalkenyl, —C₂-C₆alkynyl, —C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

R^{a} is independently, at each occurrence, —H, -D, —OH, —C₃-C₈cycloalkyl, or —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;

R^{b} is independently, at each occurrence, —H, -D, —C₁-C₆alkyl, —C₃-C₈cycloalkyl, —C₂-C₆alkenyl, or heterocyclyl containing 1-5 heteroatoms selected from the group consisting of N, S, P, and O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R³ is independently —C₁-C₆alkyl or a 3- to 12-membered monocyclic or polycyclic heterocycle, wherein each alkyl or heterocycle is optionally substituted with one or more —C₁-C₆alkyl, —OH, or —NH₂; or

R³ can combine with R^{a} to form a 3- to 12-membered monocyclic or polycyclic heterocycle or a 5- to 12-membered spiroheterocycle, wherein each heterocycle or spiroheterocycle is optionally substituted with one or more —C₁-C₆alkyl, —OH, or —NH₂;

R⁴ is independently —H, -D, or —C₁-C₆alkyl, wherein each alkyl is optionally substituted with one or more —OH, —NH₂, halogen, or oxo; or

R^{a} and R⁴, together with the atom or atoms to which they are attached, can combine to form a monocyclic or polycyclic C₃-C₁₂cycloalkyl or a monocyclic or polycyclic 3- to 12-membered heterocycle, wherein the cycloalkyl or heterocycle is optionally substituted with oxo;

R⁵ and R⁶ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, or —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently, at each occurrence, 1, 2, 3, 4, 5 or 6; and

n is independently, at each occurrence, 0, 1, 2, 3, 4, 5, 6, 7, 8, 9, or 10.

One aspect of the disclosure related to compounds of Formula I-V1:

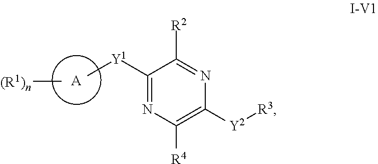

and pharmaceutically acceptable salts, prodrugs, solvates, hydrates, tautomers, or isomers thereof, wherein:

A is cycloalkyl, heterocycloalkyl, aryl, or heteroaryl, wherein cycloalkyl, heterocycloalkyl, aryl, and heteroaryl are 5- to 12-membered monocyclic or 5- to 12-membered polycyclic;

Y¹ is —S—, a direct bond, —NH—, —S(O)₂—, —S(O)₂—NH—, —C(═CH₂)—, —CH—, or —S(O)—;

Y² is —NR^{a}—, wherein the bond on the left side of Y², as drawn, is bound to the pyrazine ring and the bond on the right side of the Y² moiety, as drawn, is bound to R³;

R^{a} and R⁴, together with the atom or atoms to which they are attached, are combined to form a monocyclic or polycyclic C₃-C₁₂cycloalkyl or a monocyclic or polycyclic 3- to 12-membered heterocycle, wherein the cycloalkyl or heterocycle is optionally substituted with oxo; wherein the heterocycle optionally comprises —S(O)₂— in the heterocycle;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, —OR⁶, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, —CO₂R⁵, —C(O)NR⁵R⁶, —NR⁵C(O)R⁶, monocyclic or polycyclic heterocyclyl, spiroheterocyclyl, heteroaryl, or oxo, wherein each alkyl, alkenyl, cycloalkenyl, alkynyl, cycloalkyl, heterocyclyl, spiroheterocyclyl, or heteroaryl is optionally substituted with one or more —OH, halogen, —NO₂, oxo, ═O, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R² is independently —NH₂, —OR^{b}, —CN, —C₁-C₆alkyl, —C₂-C₆alkenyl, —C₄-C₈cycloalkenyl, —C₂-C₆alkynyl, halogen, —C(O)OR^{b}, —C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

R^{b} is independently, at each occurrence, —H, -D, —OH, —C₁-C₆alkyl, —C₃-C₈cycloalkyl, —C₂-C₆alkenyl, —(CH₂)ₙ-aryl, heterocyclyl containing 1-5 heteroatoms selected from the group consisting of N, S, P, and O, or heteroaryl containing 1-5 heteroatoms selected from the group consisting of N, S, P, and O; wherein each alkyl, cycloalkyl, alkenyl, heterocycle, heteroaryl, or —(CH₂)ₙ-aryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)NR⁵R⁶, —NR⁵C(O)R⁶, heterocycle, aryl, heteroaryl, —(CH₂)ₙOH, —C₁-C₆alkyl, —CF₃, —CHF₂, or —CH₂F;

R³ is independently —H, —C₁-C₆alkyl, a 3- to 12-membered monocyclic or polycyclic heterocycle, a 5- to 12-membered spiroheterocycle, C₃-C₈cycloalkyl, or —(CH₂)ₙ—R^{b}, wherein each alkyl, spiroheterocycle, heterocycle, or cycloalkyl is optionally substituted with one or more —C₁-C₆alkyl, —OH, —NH₂, —OR^{b}, —NHR^{b}, —(CH₂)ₙOH, heterocyclyl, or spiroheterocyclyl;

R⁵ and R⁶ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, —CF₃, or —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OR^{b}, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN; and

n is independently, at each occurrence, 0, 1, 2, 3, 4, 5, 6, 7, 8, 9, or 10.

One aspect of the disclosure related to compounds of Formula I-V2:

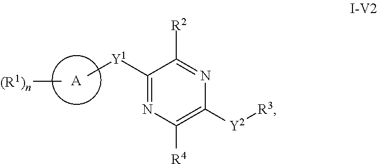

and pharmaceutically acceptable salts, prodrugs, solvates, hydrates, tautomers, and isomers thereof, wherein:

A is cycloalkyl, heterocycloalkyl, aryl, or heteroaryl, wherein cycloalkyl, heterocycloalkyl, aryl, and heteroaryl are 5- to 12-membered monocyclic or 5- to 12-membered polycyclic;

Y¹ is —S—, a direct bond, —NH—, —S(O)₂—, —S(O)₂—NH—, —C(═CH₂)—, —CH—, or —S(O)—;

Y² is —NR^{a}—, wherein the bond on the left side of Y², as drawn, is bound to the pyrazine ring and the bond on the right side of the Y² moiety, as drawn, is bound to R³;

R³ is combined with R^{a} to form a 3- to 12-membered polycyclic heterocycle or a 5- to 12-membered spiroheterocycle, wherein each heterocycle or spiroheterocycle is optionally substituted with one or more —C₁-C₆alkyl, halogen, —OH, —OR^{b}, —NH₂, —NHR^{b}, heteroaryl, heterocyclyl, —(CH₂)ₙNH₂, —(CH₂)ₙOH, —COOR^{b}, —CONHR^{b}, —CONH(CH₂)ₙCOOR^{b}, —NHCOOR^{b}, —CF₃, —CHF₂, —CH₂F, or ═O;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, —OR⁶, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, —CO₂R⁵, —C(O)NR⁵R⁶, —NR⁵C(O)R⁶, monocyclic or polycyclic heterocyclyl, spiroheterocyclyl, heteroaryl, or oxo, wherein each alkyl, alkenyl, cycloalkenyl, alkynyl, cycloalkyl, heterocyclyl, spiroheterocyclyl, or heteroaryl is optionally substituted with one or more —OH, halogen, —NO₂, oxo, ═O, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R² is independently —NH₂, —OR^{b}, —CN, —C₁-C₆alkyl, —C₂-C₆alkenyl, —C₄-C₈cycloalkenyl, —C₂-C₆alkynyl, halogen, —C(O)OR^{b}, —C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

R^{b} is independently, at each occurrence, —H, -D, —OH, —C₁-C₆alkyl, —C₃-C₈cycloalkyl, —C₂-C₆alkenyl, —(CH₂)ₙ-aryl, heterocyclyl containing 1-5 heteroatoms selected from the group consisting of N, S, P, and O, or heteroaryl containing 1-5 heteroatoms selected from the group consisting of N, S, P, and O; wherein each alkyl, cycloalkyl, alkenyl, heterocycle, heteroaryl, or —(CH₂)ₙ-aryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)NR⁵R⁶, —NR⁵C(O)R⁶, heterocycle, aryl, heteroaryl, —(CH₂)ₙOH, —C₁-C₆alkyl, —CF₃, —CHF₂, or —CH₂F;

R⁴ is independently —H, -D, —C₁-C₆alkyl, —C₁-C₆haloalkyl, —C₁-C₆hydroxyalkyl, —CF₂OH, —CHFOH, —NH—NHR⁵, —NH—OR⁵, —O—NR⁵R⁶, —NHR⁵, —OR⁵, —NHC(O)R⁵, —NHC(O)NHR⁵, —NHS(O)₂R⁵, —NHS(O)₂NHR⁵, —S(O)₂OH, —C(O)OR⁵, —NH(CH₂)ₙOH, —C(O)NH(CH₂)ₙOH, —C(O)NH(CH₂)ₙR^{b}, —C(O)R^{b}, —NH₂, —OH, —CN, —C(O)NR⁵R⁶, —S(O)₂NR⁵R⁶, C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O, wherein each alkyl, cycloalkyl, or heterocyclyl is optionally substituted with one or more —OH, —NH₂, —OR^{b}, halogen, or oxo; wherein each aryl or heteroaryl is optionally substituted with one or more —OH, —NH₂, or halogen;

R⁵ and R⁶ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, —CF₃, or —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OR^{b}, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN; and

n is independently, at each occurrence, 0, 1, 2, 3, 4, 5, 6, 7, 8, 9, or 10.

One aspect of the disclosure relates to compounds of Formula I-W:

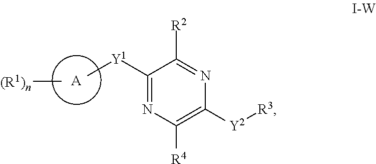

and pharmaceutically acceptable salts, prodrugs, solvates, hydrates, tautomers, and isomers thereof, wherein:

A is cycloalkyl, heterocycloalkyl, aryl, or heteroaryl, wherein cycloalkyl, heterocycloalkyl, aryl, and heteroaryl are 5- to 12-membered monocyclic or 5- to 12-membered polycyclic;

Y¹ is —S—, a direct bond, —NH—, —S(O)₂—, —S(O)₂—NH—, —C(═CH₂)—, —CH—, or —S(O)—;

Y² is —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, or —OC(O)O—; wherein the bond on the left side of Y², as drawn, is bound to the pyrazine ring and the bond on the right side of the Y² moiety, as drawn, is bound to R³;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, —OR⁶, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, —CO₂R⁵, —C(O)NR⁵R⁶, —NR⁵C(O)R⁶, monocyclic or polycyclic heterocyclyl, spiroheterocyclyl, heteroaryl, or oxo, wherein each alkyl, alkenyl, cycloalkenyl, alkynyl, cycloalkyl, heterocyclyl, spiroheterocyclyl, or heteroaryl is optionally substituted with one or more —OH, halogen, —NO₂, oxo, ═O, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R² is independently —OR^{b}, —CN, —C₁-C₆alkyl, —C₂-C₆alkenyl, —C₄-C₈cycloalkenyl, —C₂-C₆alkynyl, halogen, —C(O)OR^{b}, —C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

R^{a} is independently, at each occurrence, —H, -D, —OH, —C₃-C₈cycloalkyl, —C₁-C₆alkyl, 3- to 12-membered heterocyclyl, or —(CH₂)ₙ-aryl, wherein each alkyl or cycloalkyl is optionally substituted with one or more —NH₂, or wherein 2 R^{a}, together with the carbon atom to which they are both attached, can combine to form a 3- to 8-membered cycloalkyl;

R^{b} is independently, at each occurrence, —H, -D, —OH, —C₁-C₆alkyl, —C₃-C₈cycloalkyl, —C₂-C₆alkenyl, —(CH₂)ₙ-aryl, heterocyclyl containing 1-5 heteroatoms selected from the group consisting of N, S, P, and O, or heteroaryl containing 1-5 heteroatoms selected from the group consisting of N, S, P, and O; wherein each alkyl, cycloalkyl, alkenyl, heterocycle, heteroaryl, or —(CH₂)ₙ-aryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)NR⁵R⁶, —NR⁵C(O)R⁶, heterocycle, aryl, heteroaryl, —(CH₂)ₙOH, —C₁-C₆alkyl, —CF₃, —CHF₂, or —CH₂F;

R³ is independently —H, —C₁-C₆alkyl, a 3- to 12-membered monocyclic or polycyclic heterocycle, a 5- to 12-membered spiroheterocycle, C₃-C₈cycloalkyl, or —(CH₂)ₙ—R^{b}, wherein each alkyl, spiroheterocycle, heterocycle, or cycloalkyl is optionally substituted with one or more —C₁-C₆alkyl, —OH, —NH₂, —OR^{b}, —NHR^{b}, —(CH₂)ₙOH, heterocyclyl, or spiroheterocyclyl; or

R³ can combine with R^{a} to form a 3- to 12-membered monocyclic or polycyclic heterocycle or a 5- to 12-membered spiroheterocycle, wherein each heterocycle or spiroheterocycle is optionally substituted with one or more —C₁-C₆alkyl, halogen, —OH, —OR^{b}, —NH₂, —NHR^{b}, heteroaryl, heterocyclyl, —(CH₂)ₙNH₂, —(CH₂)ₙOH, —COOR^{b}, —CONHR^{b}, —CONH(CH₂)ₙCOOR^{b}, —NHCOOR^{b}, —CF₃, —CHF₂, —CH₂F, or ═O;

R⁴ is independently —H, -D, —C₁-C₆alkyl, —C₁-C₆haloalkyl, —C₁-C₆hydroxyalkyl —CF₂OH, —CHFOH—NH—NHR⁵, —NH—OR⁵, —O—NR⁵R⁶, —NHR⁵, —OR⁵, —NHC(O)R⁵, —NHC(O)NHR⁵, —NHS(O)₂R⁵, —NHS(O)₂NHR⁵, —S(O)₂OH, —C(O)OR⁵, —NH(CH₂)ₙOH, —C(O)NH(CH₂)ₙOH, —C(O)NH(CH₂)ₙR^{b}, —C(O)R^{b}, —NH₂, —OH, —CN, —C(O)NR⁵R⁶, —S(O)₂NR⁵R⁶, C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O, wherein each alkyl, cycloalkyl, or heterocyclyl is optionally substituted with one or more —OH, —NH₂, —OR^{b}, halogen, or oxo; wherein each aryl or heteroaryl is optionally substituted with one or more —OH, —NH₂, or halogen; or

R^{a} and R⁴, together with the atom or atoms to which they are attached, can combine to form a monocyclic or polycyclic C₃-C₁₂cycloalkyl or a monocyclic or polycyclic 3- to 12-membered heterocycle, wherein the cycloalkyl or heterocycle is optionally substituted with oxo; wherein the heterocycle optionally comprises —S(O)₂— in the heterocycle;

R⁵ and R⁶ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, —CF₃, or —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OR^{b}, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently, at each occurrence, 1, 2, 3, 4, 5 or 6; and

n is independently, at each occurrence, 0, 1, 2, 3, 4, 5, 6, 7, 8, 9, or 10.

One aspect of the disclosure relates to compounds of Formula I-X:

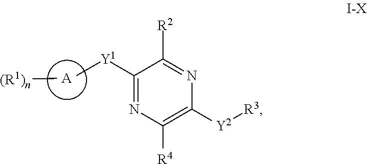

and pharmaceutically acceptable salts, prodrugs, solvates, hydrates, tautomers, or isomers thereof, wherein:

A is a 5- to 12-membered monocyclic or polycyclic cycloalkyl, heterocycloalkyl, aryl, or heteroaryl;

Y¹ is —S— or a direct bond;

Y² is —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, or —OC(O)O—; wherein the bond on the left side of Y², as drawn, is bound to the pyrazine ring and the bond on the right side of the Y² moiety, as drawn, is bound to R³;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, OXO, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R² is independently —OR^{b}, —CN, —C₁-C₆alkyl, —C₂-C₆alkenyl, —C₄-C₈cycloalkenyl, —C₂-C₆alkynyl, —C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

R^{a} is independently, at each occurrence, —H, -D, —OH, —C₃-C₈cycloalkyl, or —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;

R^{b} is independently, at each occurrence, —H, -D, —C₁-C₆alkyl, —C₃-C₈cycloalkyl, —C₂-C₆alkenyl, or heterocyclyl containing 1-5 heteroatoms selected from the group consisting of N, S, P, and O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R³ is independently —H, —C₁-C₆alkyl, or a 3- to 12-membered monocyclic or polycyclic heterocycle, wherein each alkyl or heterocycle is optionally substituted with one or more —C₁-C₆alkyl, —OH, or —NH₂; or

R³ can combine with R^{a} to form a 3- to 12-membered monocyclic or polycyclic heterocycle or a 5- to 12-membered spiroheterocycle, wherein each heterocycle or spiroheterocycle is optionally substituted with one or more —C₁-C₆alkyl, —OH, or —NH₂;

R⁴ is independently —H, -D, —C₁-C₆alkyl, —NH—NHR⁵, —NH—OR⁵, —O—NR⁵R⁶, —NHR⁵, —OR⁵, —NHC(O)R⁵, —NHC(O)NHR⁵, —NHS(O)₂R⁵, —NHS(O)₂NHR⁵, —S(O)₂OH, —C(O)OR⁵, —C(O)NR⁵R⁶, —S(O)₂NR⁵R⁶, C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O, wherein each alkyl, cycloalkyl, or heterocyclyl is optionally substituted with one or more —OH, —NH₂, halogen, or oxo; wherein each aryl or heteroaryl is optionally substituted with one or more —OH, —NH₂, or halogen; or

R^{a} and R⁴, together with the atom or atoms to which they are attached, can combine to form a monocyclic or polycyclic C₃-C₁₂cycloalkyl or a monocyclic or polycyclic 3- to 12-membered heterocycle, wherein the cycloalkyl or heterocycle is optionally substituted with oxo; wherein the heterocycle optionally comprises —S(O)₂— in the heterocycle;

R⁵ and R⁶ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, or —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently, at each occurrence, 1, 2, 3, 4, 5 or 6; and

n is independently, at each occurrence, 0, 1, 2, 3, 4, 5, 6, 7, 8, 9, or 10.

One aspect of the disclosure relates to compounds of Formula I-Y:

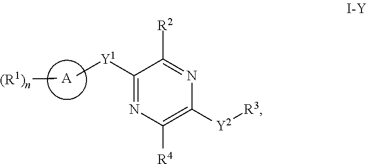

and pharmaceutically acceptable salts, prodrugs, solvates, hydrates, tautomers, or isomers thereof, wherein:

A is a 5- to 12-membered monocyclic or polycyclic cycloalkyl, heterocycloalkyl, aryl, or heteroaryl;

Y¹ is —S— or a direct bond;

Y² is —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, or —OC(O)O—; wherein the bond on the left side of Y², as drawn, is bound to the pyrazine ring and the bond on the right side of the Y² moiety, as drawn, is bound to R³;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R² is independently —OR^{b}, —CN, —C₁-C₆alkyl, —C₂-C₆alkenyl, —C₄-C₈cycloalkenyl, —C₂-C₆alkynyl, —C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

R^{a} is independently, at each occurrence, —H, -D, —OH, —C₃-C₈cycloalkyl, or —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;

R^{b} is independently, at each occurrence, —H, -D, —C₁-C₆alkyl, —C₃-C₈cycloalkyl, —C₂-C₆alkenyl, or heterocyclyl containing 1-5 heteroatoms selected from the group consisting of N, S, P, and O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, heteroaryl, —(CH₂)ₙOH, —C₁-C₆alkyl, —CF₃, —CHF₂, or —CH₂F;

R³ is independently —H, —C₁-C₆alkyl, a 3- to 12-membered monocyclic or polycyclic heterocycle, C₃-C₈cycloalkyl, or —(CH₂)ₙ—R^{b}, wherein each alkyl, heterocycle, or cycloalkyl is optionally substituted with one or more —C₁-C₆alkyl, —OH, —NH₂, —OR^{b}, —NHR^{b}, —(CH₂)ₙOH, heterocyclyl, or spiroheterocyclyl; or

R³ can combine with R^{a} to form a 3- to 12-membered monocyclic or polycyclic heterocycle or a 5- to 12-membered spiroheterocycle, wherein each heterocycle or spiroheterocycle is optionally substituted with one or more —C₁-C₆alkyl, —OH, —NH₂, heteroaryl, heterocyclyl, —(CH₂)ₙNH₂, —COOR^{b}, —CONHR^{b}, —CONH(CH₂)ₙCOOR^{b}, —NHCOOR^{b}, —CF₃, —CHF₂, or —CH₂F;

R⁴ is independently —H, -D, —C₁-C₆alkyl, —NH—NHR⁵, —NH—OR⁵, —O—NR⁵R⁶, —NHR⁵, —OR⁵, —NHC(O)R⁵, —NHC(O)NHR⁵, —NHS(O)₂R⁵, —NHS(O)₂NHR⁵, —S(O)₂OH, —C(O)OR⁵, —NH(CH₂)ₙOH, —C(O)NH(CH₂)ₙOH, —C(O)NH(CH₂)ₙR^{b}, —C(O)R^{b}, —NH₂, —OH, —CN, —C(O)NR⁵R⁶, —S(O)₂NR⁵R⁶, C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O, wherein each alkyl, cycloalkyl, or heterocyclyl is optionally substituted with one or more —OH, —NH₂, halogen, or oxo; wherein each aryl or heteroaryl is optionally substituted with one or more —OH, —NH₂, or halogen; or

R^{a} and R⁴, together with the atom or atoms to which they are attached, can combine to form a monocyclic or polycyclic C₃-C₁₂cycloalkyl or a monocyclic or polycyclic 3- to 12-membered heterocycle, wherein the cycloalkyl or heterocycle is optionally substituted with oxo; wherein the heterocycle optionally comprises —S(O)₂— in the heterocycle;

R⁵ and R⁶ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, or —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently, at each occurrence, 1, 2, 3, 4, 5 or 6; and

n is independently, at each occurrence, 0, 1, 2, 3, 4, 5, 6, 7, 8, 9, or 10.

One aspect of the disclosure relates to compounds of Formula I-Z:

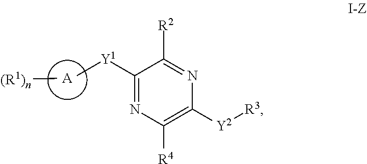

and pharmaceutically acceptable salts, prodrugs, solvates, hydrates, tautomers, or isomers thereof, wherein:

A is a 5- to 12-membered monocyclic or polycyclic cycloalkyl, heterocycloalkyl, aryl, or heteroaryl;

Y¹ is —S—, a direct bond, —NH—, —S(O)₂—, —S(O)₂—NH—, —C(═CH₂)—, —CH—, or —S(O)—;

Y² is —NR^{a}—, —(CR^{a}₂)ₘ—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —OC(O)N(R^{a})—, —N(R^{a})C(O)O—, —C(O)N(R^{a})O—, —N(R^{a})C(S)—, or —C(S)N(R^{a})—; wherein the bond on the left side of Y², as drawn, is bound to the pyrazine ring and the bond on the right side of the Y² moiety, as drawn, is bound to R³;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, OXO, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R² is independently —OR^{b}, —NH₂, —CN, —C₁-C₆alkyl, —C₂-C₆alkenyl, —C₄-C₈cycloalkenyl, —C₂-C₆alkynyl, halogen, —C(O)OR^{b}, —C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

R^{a} is independently, at each occurrence —OH, —C₃-C₈cycloalkyl, or —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;

R^{b} is independently, at each occurrence, —H, -D, —C₁-C₆alkyl, —C₃-C₈cycloalkyl, —C₂-C₆alkenyl, or heterocyclyl containing 1-5 heteroatoms selected from the group consisting of N, S, P, and O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, heteroaryl, —(CH₂)ₙOH, —C₁-C₆alkyl, —CF₃, —CHF₂, or —CH₂F;

R³ is independently —H, —C₁-C₆alkyl, a 3- to 12-membered monocyclic or polycyclic heterocycle, C₃-C₈cycloalkyl, or —(CH₂)ₙ—R^{b}, wherein each alkyl, heterocycle, or cycloalkyl is optionally substituted with one or more —C₁-C₆alkyl, —OH, —NH₂, —OR^{b}, —NHR^{b}, —(CH₂)ₙOH, heterocyclyl, or spiroheterocyclyl; or

R³ can combine with R^{a} to form a 3- to 12-membered monocyclic or polycyclic heterocycle or a 5- to 12-membered spiroheterocycle, wherein each heterocycle or spiroheterocycle is optionally substituted with one or more —C₁-C₆alkyl, —OH, —NH₂, heteroaryl, heterocyclyl, —(CH₂)ₙNH₂, —COOR^{b}, —CONHR^{b}, —CONH(CH₂)ₙCOOR^{b}, —NHCOOR^{b}, —CF₃, —CHF₂, or —CH₂F;

R⁴ is independently —C₁-C₆alkyl, —NH—NHR⁵, —NH—OR⁵, —O—NR⁵R⁶, —NHR⁵, —OR⁵, —NHC(O)R⁵, —NHC(O)NHR⁵, —NHS(O)₂R⁵, —NHS(O)₂NHR⁵, —S(O)₂OH, —C(O)OR⁵, —NH(CH₂)ₙOH, —C(O)NH(CH₂)ₙOH, —C(O)NH(CH₂)ₙR^{b}, —C(O)R^{b}, —NH₂, —OH, —C(O)NR⁵R⁶, —S(O)₂NR⁵R⁶, C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O, wherein each alkyl, cycloalkyl, or heterocyclyl is optionally substituted with one or more —OH, —NH₂, halogen, or oxo; wherein each aryl or heteroaryl is optionally substituted with one or more —OH, —NH₂, or halogen;

R^{a} and R⁴, together with the atom or atoms to which they are attached, are combined to form a monocyclic or polycyclic C₃-C₁₂cycloalkyl or a monocyclic or polycyclic 3- to 12-membered heterocycle, wherein the cycloalkyl or heterocycle is optionally substituted with oxo; wherein the heterocycle optionally comprises —S(O)₂— in the heterocycle;

R⁵ and R⁶ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, or —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently, at each occurrence, 1, 2, 3, 4, 5 or 6; and

n is independently, at each occurrence, 0, 1, 2, 3, 4, 5, 6, 7, 8, 9, or 10.

One aspect of the invention relates to compounds of Formula IV:

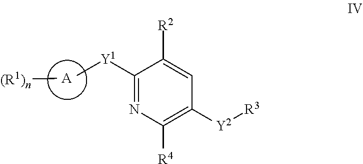

and pharmaceutically acceptable salts, prodrugs, solvates, hydrates, tautomers, or isomers thereof, wherein:

A is selected from the group consisting of 5- to 12-membered monocyclic or polycyclic cycloalkyl, heterocycloalkyl, aryl, or heteroaryl;

Y¹ is —S— or a direct bond;

Y² is selected from the group consisting of: —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the pyridine ring and the bond on the right side of the Y² moiety is bound to R³;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R² is independently —OR^{b}, —CN, —C₁-C₆alkyl, —C₂-C₆alkenyl, —C₄-C₈cycloalkenyl, —C₂-C₆alkynyl, —C₃-C₈cycloalkyl, aryl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

R^{a} is independently, at each occurrence, selected from the group consisting of —H, -D, —OH, —C₃-C₈cycloalkyl, and —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;

R^{b} is independently —H, -D, —C₁-C₆alkyl, —C₁-C₆cycloalkyl, —C₂-C₆alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R³ is independently, at each occurrence, selected from the group consisting of —C₁-C₆alkyl, or a 3- to 12-membered monocyclic or polycyclic heterocycle, wherein each alkyl or heterocycle is optionally substituted with one or more —C₁-C₆alkyl, —OH, or —NH₂; or

R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆alkyl, —OH, or —NH₂;

R⁴ is independently, at each occurrence, —H, -D, or —C₁-C₆alkyl, wherein each alkyl is optionally substituted with one or more —OH, —NH₂, halogen, or oxo; or

R^{a} and R⁴, together with the atom or atoms to which they are attached, can combine to form a monocyclic or polycyclic C₃-C₁₂cycloalkyl, or a monocyclic or polycyclic 3- to 12-membered heterocycle, wherein the cycloalkyl or heterocycle is optionally substituted with oxo;

R⁵ and R⁶ are each independently, at each occurrence, selected from the group consisting of —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, and —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently 1, 2, 3, 4, 5 or 6; and

n is independently 0, 1, 2, 3, 4, 5, 6, 7, 8, 9, or 10.

Another aspect of the invention relates to compounds of Formula V:

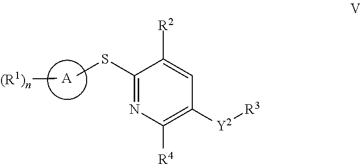

and pharmaceutically acceptable salts, prodrugs, solvates, hydrates, tautomers, or isomers thereof, wherein:

A is selected from the group consisting of 5- to 12-membered monocyclic or polycyclic cycloalkyl, heterocycloalkyl, aryl, or heteroaryl;

Y² is selected from the group consisting of: —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the pyridine ring and the bond on the right side of the Y² moiety is bound to R³;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R² is independently —OR^{b}, —CN, —C₁-C₆alkyl, —C₂-C₆alkenyl, —C₄-C₈cycloalkenyl, —C₂-C₆alkynyl, —C₃-C₈cycloalkyl, aryl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

R^{a} is independently, at each occurrence, selected from the group consisting of —H, -D, —OH, —C₃-C₈cycloalkyl, and —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;

R^{b} is independently —H, -D, —C₁-C₆alkyl, —C₁-C₆cycloalkyl, —C₂-C₆alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R³ is independently, at each occurrence, selected from the group consisting of —C₁-C₆alkyl, or a 3- to 12-membered monocyclic or polycyclic heterocycle, wherein each alkyl or heterocycle is optionally substituted with one or more —C₁-C₆alkyl, —OH, or —NH₂; or

R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆alkyl, —OH, or —NH₂;

R⁴ is independently, at each occurrence, —H, -D, or —C₁-C₆alkyl, wherein each alkyl is optionally substituted with one or more —OH, —NH₂, halogen, or oxo; or

R^{a} and R⁴, together with the atom or atoms to which they are attached, can combine to form a monocyclic or polycyclic C₃-C₁₂cycloalkyl, or a monocyclic or polycyclic 3- to 12-membered heterocycle, wherein the cycloalkyl or heterocycle is optionally substituted with oxo;

R⁵ and R⁶ are each independently, at each occurrence, selected from the group consisting of —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, and —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently 1, 2, 3, 4, 5 or 6; and

n is independently 0, 1, 2, 3, 4, 5, 6, 7, 8, 9, or 10.

Another aspect of the invention relates to compounds of Formula VI:

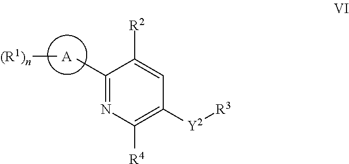

and pharmaceutically acceptable salts, prodrugs, solvates, hydrates, tautomers, or isomers thereof, wherein:

A is selected from the group consisting of 5- to 12-membered monocyclic or polycyclic cycloalkyl, heterocycloalkyl, aryl, or heteroaryl;

Y² is selected from the group consisting of: —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the pyridine ring and the bond on the right side of the Y² moiety is bound to R³;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R² is independently —OR^{b}, —CN, —C₁-C₆alkyl, —C₂-C₆alkenyl, —C₄-C₈cycloalkenyl, —C₂-C₆alkynyl, —C₃-C₈cycloalkyl, aryl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

R^{a} is independently, at each occurrence, selected from the group consisting of —H, -D, —OH, —C₃-C₈cycloalkyl, and —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;

R^{b} is independently —H, -D, —C₁-C₆alkyl, —C₁-C₆cycloalkyl, —C₂-C₆alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R³ is independently, at each occurrence, selected from the group consisting of —C₁-C₆alkyl, or a 3- to 12-membered monocyclic or polycyclic heterocycle, wherein each alkyl or heterocycle is optionally substituted with one or more —C₁-C₆alkyl, —OH, or —NH₂; or

R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆alkyl, —OH, or —NH₂;

R⁴ is independently, at each occurrence, —H, -D, or —C₁-C₆alkyl, wherein each alkyl is optionally substituted with one or more —OH, —NH₂, halogen, or oxo; or

R^{a} and R⁴, together with the atom or atoms to which they are attached, can combine to form a monocyclic or polycyclic C₃-C₁₂cycloalkyl, or a monocyclic or polycyclic 3- to 12-membered heterocycle, wherein the cycloalkyl or heterocycle is optionally substituted with oxo;

R⁵ and R⁶ are each independently, at each occurrence, selected from the group consisting of —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, and —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently 1, 2, 3, 4, 5 or 6; and

n is independently 0, 1, 2, 3, 4, 5, 6, 7, 8, 9, or 10.

One aspect of the invention relates to compounds of Formula IV-Y:

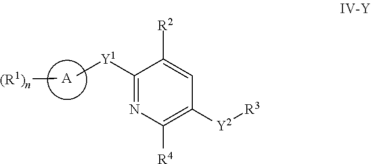

or a pharmaceutically acceptable salt, prodrug, solvate, hydrate, tautomer, or isomer thereof, wherein:

A is selected from the group consisting of 5- to 12-membered monocyclic or polycyclic cycloalkyl, heterocycloalkyl, aryl, or heteroaryl;

Y¹ is —S— or a direct bond;

Y² is selected from the group consisting of: —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the pyridine ring and the bond on the right side of the Y² moiety, as drawn, is bound to R³;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R² is independently —OR^{b}, —CN, —C₁-C₆alkyl, —C₂-C₆alkenyl, —C₄-C₈cycloalkenyl, —C₂-C₆alkynyl, —C₃-C₈cycloalkyl, aryl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

R^{a} is independently, at each occurrence, selected from the group consisting of —H, -D, —OH, —C₃-C₈cycloalkyl, and —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;

R^{b} is independently —H, -D, —C₁-C₆alkyl, —C₁-C₆cycloalkyl, —C₂-C₆alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, heteroaryl, —(CH₂)ₙOH, —C₁-C₆alkyl, CF₃, CHF₂, or CH₂F;

R³ is independently, at each occurrence, selected from the group consisting of —H, —C₁-C₆alkyl, a 3- to 12-membered monocyclic or polycyclic heterocycle, C₃-C₈cycloalkyl, or —(CH₂)ₙ—R^{b}, wherein each alkyl, heterocycle, or cycloalkyl is optionally substituted with one or more —C₁-C₆alkyl, —OH, —NH₂, —OR^{a}, —NHR^{a}, —(CH₂)ₙOH, heterocyclyl, or spiroheterocyclyl; or

R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆alkyl, —OH, —NH₂, heteroaryl, heterocyclyl, —(CH₂)ₙNH₂, —COOR^{a}, —CONHR^{b}, —CONH(CH₂)ₙCOOR^{a}, —NHCOOR³, —CF₃, CHF₂, or CH₂F;

R⁴ is independently, at each occurrence, —H, -D, —C₁-C₆alkyl, —NH—NHR⁵, —NH—OR⁵, —O—NR⁵R⁶, —NHR⁵, —OR⁵, —NHC(O)R⁵, —NHC(O)NHR⁵, —NHS(O)₂R⁵, —NHS(O)₂NHR⁵, —S(O)₂OH, —C(O)OR⁵, —NH(CH₂)ₙOH, —C(O)NH(CH₂)ₙOH, —C(O)NH(CH₂)ₙR^{b}, —C(O)R^{b}, NH₂, —OH, —CN, —C(O)NR⁵R⁶, —S(O)₂NR⁵R⁶, C₃-C₈cycloalkyl, aryl, heterocyclyl containing 1-5 heteroatoms selected from the group consisting of N, S, P, or O, heteroaryl containing 1-5 heteroatoms selected from the group consisting of N, S, P, or O, wherein each alkyl, cycloalkyl, or heterocyclyl is optionally substituted with one or more —OH, —NH₂, halogen, or oxo; wherein each aryl or heteroaryl is optionally substituted with one or more —OH, —NH₂, or halogen; or

R^{a} and R⁴, together with the atom or atoms to which they are attached, can combine to form a monocyclic or polycyclic C₃-C₁₂cycloalkyl, or a monocyclic or polycyclic 3- to 12-membered heterocycle, wherein the cycloalkyl or heterocycle is optionally substituted with oxo; wherein the heterocycle optionally comprises —S(O)₂— in the heterocycle;

R⁵ and R⁶ are each independently, at each occurrence, selected from the group consisting of —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, and —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently 1, 2, 3, 4, 5 or 6; and

n is independently 0, 1, 2, 3, 4, 5, 6, 7, 8, 9, or 10.

One aspect of the invention relates to compounds of Formula IV-Z:

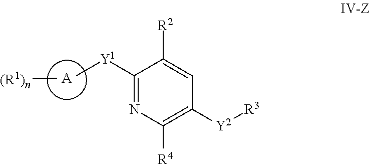

or a pharmaceutically acceptable salt, prodrug, solvate, hydrate, tautomer, or isomer thereof, wherein:

A is selected from the group consisting of 5- to 12-membered monocyclic or polycyclic cycloalkyl, heterocycloalkyl, aryl, or heteroaryl;

Y¹ is —S—, a direct bond, —NH—, —S(O)₂—, —S(O)₂—NH—, —C(═CH₂)—, —CH—, or —S(O)—;

Y² is selected from the group consisting of: —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the pyridine ring and the bond on the right side of the Y² moiety, as drawn, is bound to R³;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R² is independently —OR^{b}, —CN, —C₁-C₆alkyl, —C₂-C₆alkenyl, —C₄-C₈cycloalkenyl, —C₂-C₆alkynyl, —NH₂, halogen, —C(O)OR^{a}, —C₃-C₈cycloalkyl, aryl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

R^{a} is independently, at each occurrence, selected from the group consisting of —H, -D, —OH, —C₃-C₈cycloalkyl, and —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;

R^{b} is independently —H, -D, —C₁-C₆alkyl, —C₁-C₆cycloalkyl, —C₂-C₆alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, heteroaryl, —(CH₂)ₙOH, —C₁-C₆alkyl, CF₃, CHF₂, or CH₂F;

R³ is independently, at each occurrence, selected from the group consisting of —H, —C₁-C₆alkyl, a 3- to 12-membered monocyclic or polycyclic heterocycle, C₃-C₈cycloalkyl, or —(CH₂)ₙ—R^{b}, wherein each alkyl, heterocycle, or cycloalkyl is optionally substituted with one or more —C₁-C₆alkyl, —OH, —NH₂, —OR^{a}, —NHR^{a}, —(CH₂)ₙOH, heterocyclyl, or spiroheterocyclyl; or

R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆alkyl, —OH, —NH₂, heteroaryl, heterocyclyl, —(CH₂)ₙNH₂, —COOR^{a}, —CONHR^{b}, —CONH(CH₂)ₙCOOR^{a}, —NHCOOR³, —CF₃, CHF₂, or CH₂F;

R⁴ is independently, at each occurrence, —H, -D, —C₁-C₆alkyl, —NH—NHR⁵, —NH—OR⁵, —O—NR⁵R⁶, —NHR⁵, —OR⁵, —NHC(O)R⁵, —NHC(O)NHR⁵, —NHS(O)₂R⁵, —NHS(O)₂NHR⁵, —S(O)₂OH, —C(O)OR⁵, —NH(CH₂)ₙOH, —C(O)NH(CH₂)ₙOH, —C(O)NH(CH₂)ₙR^{b}, —C(O)R^{b}, NH₂, —OH, —CN, —C(O)NR⁵R⁶, —S(O)₂NR⁵R⁶, C₃-C₈cycloalkyl, aryl, heterocyclyl containing 1-5 heteroatoms selected from the group consisting of N, S, P, or O, heteroaryl containing 1-5 heteroatoms selected from the group consisting of N, S, P, or O, wherein each alkyl, cycloalkyl, or heterocyclyl is optionally substituted with one or more —OH, —NH₂, halogen, or oxo; wherein each aryl or heteroaryl is optionally substituted with one or more —OH, —NH₂, or halogen; or

R^{a} and R⁴, together with the atom or atoms to which they are attached, can combine to form a monocyclic or polycyclic C₃-C₁₂cycloalkyl, or a monocyclic or polycyclic 3- to 12-membered heterocycle, wherein the cycloalkyl or heterocycle is optionally substituted with oxo; wherein the heterocycle optionally comprises —S(O)₂— in the heterocycle;

R⁵ and R⁶ are each independently, at each occurrence, selected from the group consisting of —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, and —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently 1, 2, 3, 4, 5 or 6; and

n is independently 0, 1, 2, 3, 4, 5, 6, 7, 8, 9, or 10.

One aspect of the invention relates to compounds of Formula VII:

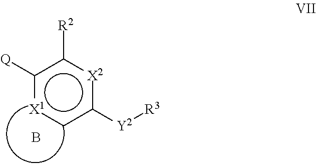

and pharmaceutically acceptable salts, prodrugs, solvates, hydrates, tautomers, or isomers thereof, wherein:

Q is H or

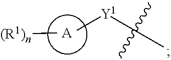

A is selected from the group consisting of 5- to 12-membered monocyclic or polycyclic cycloalkyl, heterocycloalkyl, aryl, or heteroaryl;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

Y¹ is —S—, a direct bond, —NH—, —S(O)₂—, —S(O)₂—NH—, —C(═CH₂)—, —CH—, or —S(O)—;

X¹ is N or C;

X² is N or CH;

B, including the atoms at the points of attachment, is a monocyclic or polycyclic 5- to 12-membered heterocycle or a monocyclic or polycyclic 5- to 12-membered heteroaryl;

R² is independently H, —OR^{b}, —NR⁵R⁶, —CN, —C₁-C₆alkyl, —C₂-C₆alkenyl, —C₄-C₈cycloalkenyl, —C₂-C₆alkynyl, —NH₂, halogen, —C(O)OR^{a}, —C₃-C₈cycloalkyl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

Y² is selected from the group consisting of: —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the ring and the bond on the right side of the Y² moiety, as drawn, is bound to R³;

R^{a} is independently, at each occurrence, selected from the group consisting of —H, -D, —OH, —C₃-C₈cycloalkyl, and —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;

R^{b} is independently —H, -D, —C₁-C₆alkyl, —C₁-C₆cycloalkyl, —C₂-C₆alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, heteroaryl, —(CH₂)ₙOH, —C₁-C₆alkyl, CF₃, CHF₂, or CH₂F;

R³ is independently, at each occurrence, selected from the group consisting of —H, —C₁-C₆alkyl, a 3- to 12-membered monocyclic or polycyclic heterocycle, C₃-C₈cycloalkyl, or —(CH₂)ₙ—R^{b}, wherein each alkyl, heterocycle, or cycloalkyl is optionally substituted with one or more —C₁-C₆alkyl, —OH, —NH₂, —OR^{a}, —NHR^{a}, —(CH₂)ₙOH, heterocyclyl, or spiroheterocyclyl; or

R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆alkyl, —OH, —NH₂, heteroaryl, heterocyclyl, —(CH₂)ₙNH₂, —COOR^{a}, —CONHR^{b}, —CONH(CH₂)ₙCOOR^{a}, —NHCOOR³, —CF₃, CHF₂, or CH₂F;

R⁵ and R⁶ are each independently, at each occurrence, selected from the group consisting of —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, and —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently 1, 2, 3, 4, 5 or 6; and

n is independently 0, 1, 2, 3, 4, 5, 6, 7, 8, 9, or 10.

Another aspect of the invention relates to compounds of Formula VIII:

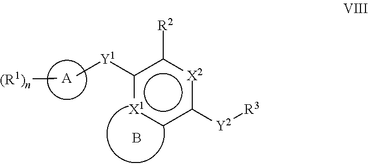

and pharmaceutically acceptable salts, prodrugs, solvates, hydrates, tautomers, or isomers thereof, wherein:

A is selected from the group consisting of 5- to 12-membered monocyclic or polycyclic cycloalkyl, heterocycloalkyl, aryl, or heteroaryl;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

Y¹ is —S—, a direct bond, —NH—, —S(O)₂—, —S(O)₂—NH—, —C(═CH₂)—, —CH—, or —S(O)—;

X¹ is N or C;

X² is N or CH;

B, including the atoms at the points of attachment, is a monocyclic or polycyclic 5- to 12-membered heterocycle or a monocyclic or polycyclic 5- to 12-membered heteroaryl;

R² is independently H, —OR^{b}, —NR⁵R⁶, —CN, —C₁-C₆alkyl, —C₂-C₆alkenyl, —C₄-C₈cycloalkenyl, —C₂-C₆alkynyl, —NH₂, halogen, —C(O)OR^{a}, —C₃-C₈cycloalkyl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

Y² is selected from the group consisting of: —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the ring and the bond on the right side of the Y² moiety, as drawn, is bound to R³;

R^{a} is independently, at each occurrence, selected from the group consisting of —H, -D, —OH, —C₃-C₈cycloalkyl, and —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;

R^{b} is independently —H, -D, —C₁-C₆alkyl, —C₁-C₆cycloalkyl, —C₂-C₆alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, heteroaryl, —(CH₂)ₙOH, —C₁-C₆alkyl, CF₃, CHF₂, or CH₂F;

R³ is independently, at each occurrence, selected from the group consisting of —H, —C₁-C₆alkyl, a 3- to 12-membered monocyclic or polycyclic heterocycle, C₃-C₈cycloalkyl, or —(CH₂)ₙ—R^{b}, wherein each alkyl, heterocycle, or cycloalkyl is optionally substituted with one or more —C₁-C₆alkyl, —OH, —NH₂, —OR^{a}, —NHR^{a}, —(CH₂)ₙOH, heterocyclyl, or spiroheterocyclyl; or

R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆alkyl, —OH, —NH₂, heteroaryl, heterocyclyl, —(CH₂)ₙNH₂, —COOR^{a}, —CONHR^{b}, —CONH(CH₂)ₙCOOR^{a}, —NHCOOR³, —CF₃, CHF₂, or CH₂F;

R⁵ and R⁶ are each independently, at each occurrence, selected from the group consisting of —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, and —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently 1, 2, 3, 4, 5 or 6; and

n is independently 0, 1, 2, 3, 4, 5, 6, 7, 8, 9, or 10.

Another aspect of the invention relates to compounds of Formula IX:

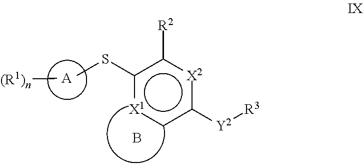

and pharmaceutically acceptable salts, prodrugs, solvates, hydrates, tautomers, or isomers thereof, wherein:

A is selected from the group consisting of 5- to 12-membered monocyclic or polycyclic cycloalkyl, heterocycloalkyl, aryl, or heteroaryl;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

X¹ is N or C;

X² is N or CH;

B, including the atoms at the points of attachment, is a monocyclic or polycyclic 5- to 12-membered heterocycle or a monocyclic or polycyclic 5- to 12-membered heteroaryl;

R² is independently H, —OR^{b}, —NR⁵R⁶, —CN, —C₁-C₆alkyl, —C₂-C₆alkenyl, —C₄-C₈cycloalkenyl, —C₂-C₆alkynyl, —NH₂, halogen, —C(O)OR^{a}, —C₃-C₈cycloalkyl, aryl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

Y² is selected from the group consisting of: —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the ring and the bond on the right side of the Y² moiety, as drawn, is bound to R³;

R^{a} is independently, at each occurrence, selected from the group consisting of —H, -D, —OH, —C₃-C₈cycloalkyl, and —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;

R^{b} is independently —H, -D, —C₁-C₆alkyl, —C₁-C₆cycloalkyl, —C₂-C₆alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, heteroaryl, —(CH₂)ₙOH, —C₁-C₆alkyl, CF₃, CHF₂, or CH₂F;

R³ is independently, at each occurrence, selected from the group consisting of —H, —C₁-C₆alkyl, a 3- to 12-membered monocyclic or polycyclic heterocycle, C₃-C₈cycloalkyl, or —(CH₂)ₙ—R^{b}, wherein each alkyl, heterocycle, or cycloalkyl is optionally substituted with one or more —C₁-C₆alkyl, —OH, —NH₂, —OR^{a}, —NHR^{a}, —(CH₂)ₙOH, heterocyclyl, or spiroheterocyclyl; or

R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆alkyl, —OH, —NH₂, heteroaryl, heterocyclyl, —(CH₂)ₙNH₂, —COOR^{a}, —CONHR^{b}, —CONH(CH₂)ₙCOOR^{a}, —NHCOOR³, —CF₃, CHF₂, or CH₂F;

R⁵ and R⁶ are each independently, at each occurrence, selected from the group consisting of —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, and —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently 1, 2, 3, 4, 5 or 6; and

n is independently 0, 1, 2, 3, 4, 5, 6, 7, 8, 9, or 10.

Another aspect of the invention relates to compounds of Formula X:

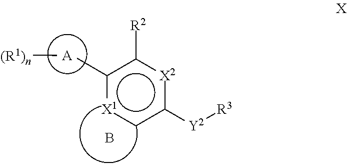

and pharmaceutically acceptable salts, prodrugs, solvates, hydrates, tautomers, or isomers thereof, wherein:

A is selected from the group consisting of 5- to 12-membered monocyclic or polycyclic cycloalkyl, heterocycloalkyl, aryl, or heteroaryl;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

X¹ is N or C;

X² is N or CH;

B, including the atoms at the points of attachment, is a monocyclic or polycyclic 5- to 12-membered heterocycle or a monocyclic or polycyclic 5- to 12-membered heteroaryl;

R² is independently H, —OR^{b}, —NR⁵R⁶, —CN, —C₁-C₆alkyl, —C₂-C₆alkenyl, —C₄-C₈cycloalkenyl, —C₂-C₆alkynyl, —NH₂, halogen, —C(O)OR^{a}, —C₃-C₈cycloalkyl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

Y² is selected from the group consisting of: —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the ring and the bond on the right side of the Y² moiety, as drawn, is bound to R³;

R^{a} is independently, at each occurrence, selected from the group consisting of —H, -D, —OH, —C₃-C₈cycloalkyl, and —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;

R^{b} is independently —H, -D, —C₁-C₆alkyl, —C₁-C₆cycloalkyl, —C₂-C₆alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, heteroaryl, —(CH₂)ₙOH, —C₁-C₆alkyl, CF₃, CHF₂, or CH₂F;

R³ is independently, at each occurrence, selected from the group consisting of —H, —C₁-C₆alkyl, a 3- to 12-membered monocyclic or polycyclic heterocycle, C₃-C₈cycloalkyl, or —(CH₂)ₙ—R^{b}, wherein each alkyl, heterocycle, or cycloalkyl is optionally substituted with one or more —C₁-C₆alkyl, —OH, —NH₂, —OR^{a}, —NHR^{a}, —(CH₂)ₙOH, heterocyclyl, or spiroheterocyclyl; or

R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆alkyl, —OH, —NH₂, heteroaryl, heterocyclyl, —(CH₂)ₙNH₂, —COOR^{a}, —CONHR^{b}, —CONH(CH₂)ₙCOOR^{a}, —NHCOOR³, —CF₃, CHF₂, or CH₂F;

R⁵ and R⁶ are each independently, at each occurrence, selected from the group consisting of —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, and —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently 1, 2, 3, 4, 5 or 6; and

n is independently 0, 1, 2, 3, 4, 5, 6, 7, 8, 9, or 10.

Another aspect of the present disclosure relates to compounds, and pharmaceutically acceptable salts, prodrugs, solvates, hydrates, tautomers, or isomers thereof, in Table 1.

TABLE 1

| Cmp | |
|---|---|
| d# | Structure |
| 1 | 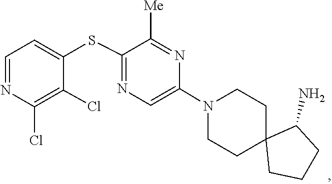 [SMILES: Cc1nc(N2CCC3(CCC[C@H]3N)CC2)cnc1Sc1ccnc(Cl)c1Cl] |
| 2 | 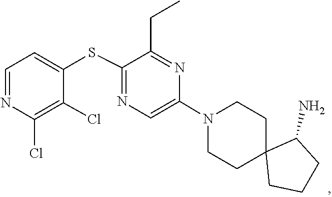 [SMILES: CCc1nc(N2CCC3(CCC[C@H]3N)CC2)cnc1Sc1ccnc(Cl)c1Cl] |
| 3 | 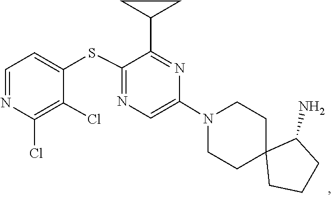 [SMILES: N[C@@H]1CCCC12CCN(c1cnc(Sc3ccnc(Cl)c3Cl)c(C3CC3)n1)CC2] |
| 4 | 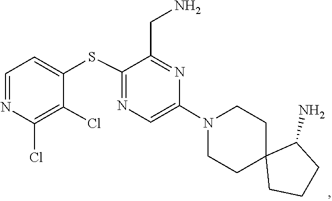 [SMILES: NCc1nc(N2CCC3(CCC[C@H]3N)CC2)cnc1Sc1ccnc(Cl)c1Cl] |
| 5 | 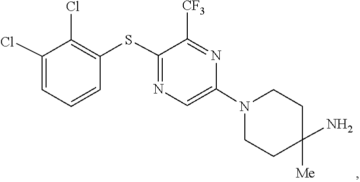 [SMILES: CC1(N)CCN(c2cnc(Sc3cccc(Cl)c3Cl)c(C(F)(F)F)n2)CC1] |
| 6 | 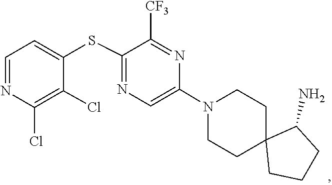 [SMILES: N[C@@H]1CCCC12CCN(c1cnc(Sc3ccnc(Cl)c3Cl)c(C(F)(F)F)n1)CC2] |
| 7 | 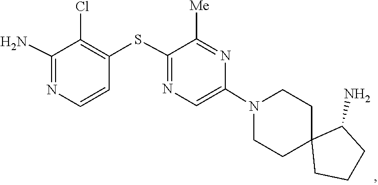 [SMILES: Cc1nc(N2CCC3(CCC[C@H]3N)CC2)cnc1Sc1ccnc(N)c1Cl] |
| 8 | 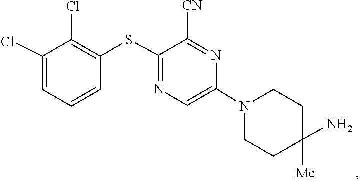 [SMILES: CC1(N)CCN(c2cnc(Sc3cccc(Cl)c3Cl)c(C#N)n2)CC1] |
| 9 | 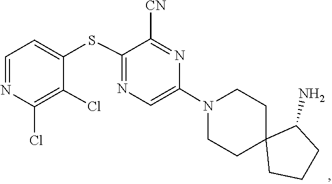 [SMILES: N#Cc1nc(N2CCC3(CCC[C@H]3N)CC2)cnc1Sc1ccnc(Cl)c1Cl] |
| 10 | 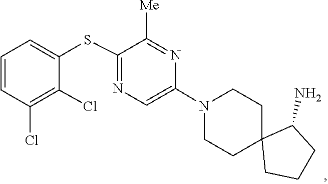 [SMILES: Cc1nc(N2CCC3(CCC[C@H]3N)CC2)cnc1Sc1cccc(Cl)c1Cl] |
| 11 | 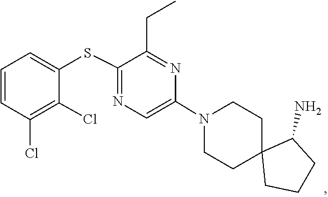 [SMILES: CCc1nc(N2CCC3(CCC[C@H]3N)CC2)cnc1Sc1cccc(Cl)c1Cl] |
| 12 | 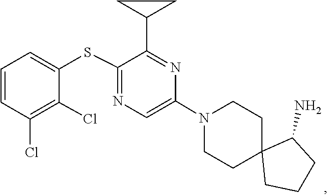 [SMILES: N[C@@H]1CCCC12CCN(c1cnc(Sc3cccc(Cl)c3Cl)c(C3CC3)n1)CC2] |
| 13 | 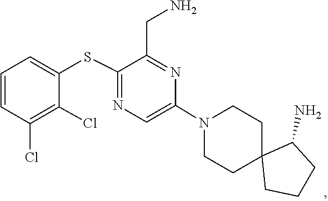 [SMILES: NCc1nc(N2CCC3(CCC[C@H]3N)CC2)cnc1Sc1cccc(Cl)c1Cl] |
| 14 | 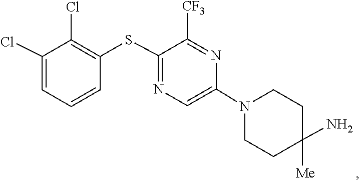 [SMILES: CC1(N)CCN(c2cnc(Sc3cccc(Cl)c3Cl)c(C(F)(F)F)n2)CC1] |
| 15 | 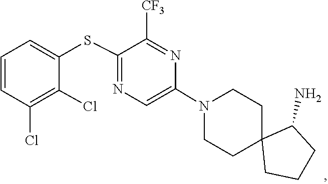 [SMILES: N[C@@H]1CCCC12CCN(c1cnc(Sc3cccc(Cl)c3Cl)c(C(F)(F)F)n1)CC2] |
| 16 | 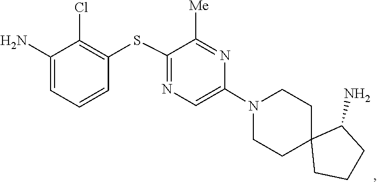 [SMILES: Cc1nc(N2CCC3(CCC[C@H]3N)CC2)cnc1Sc1cccc(N)c1Cl] |
| 17 | 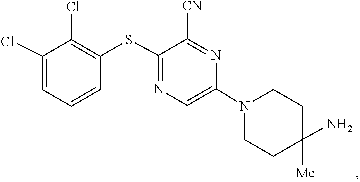 [SMILES: CC1(N)CCN(c2cnc(Sc3cccc(Cl)c3Cl)c(C#N)n2)CC1] |
| 18 | 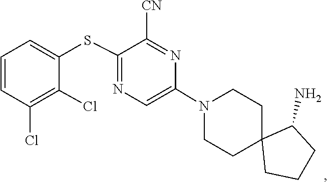 [SMILES: N#Cc1nc(N2CCC3(CCC[C@H]3N)CC2)cnc1Sc1cccc(Cl)c1Cl] |
| 19 | 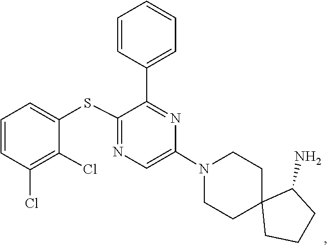 [SMILES: N[C@@H]1CCCC12CCN(c1cnc(Sc3cccc(Cl)c3Cl)c(-c3ccccc3)n1)CC2] |
| 20 | 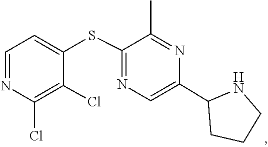 [SMILES: Cc1nc(C2CCCN2)cnc1Sc1ccnc(Cl)c1Cl] |
| 21 | 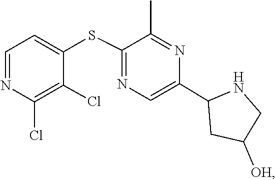 [SMILES: Cc1nc(C2CC(O)CN2)cnc1Sc1ccnc(Cl)c1Cl] |
| 22 | 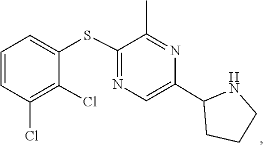 [SMILES: Cc1nc(C2CCCN2)cnc1Sc1cccc(Cl)c1Cl] |
| 23 | 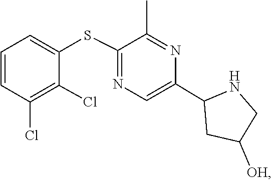 [SMILES: Cc1nc(C2CC(O)CN2)cnc1Sc1cccc(Cl)c1Cl] |
| 24 | 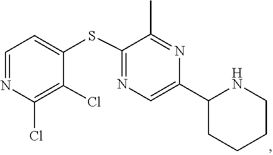 [SMILES: Cc1nc(C2CCCCN2)cnc1Sc1ccnc(Cl)c1Cl] |
| 25 | 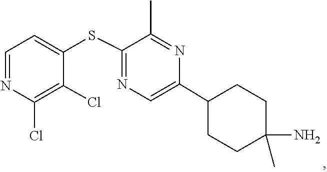 [SMILES: Cc1nc(C2CCC(C)(N)CC2)cnc1Sc1ccnc(Cl)c1Cl] |
| 26 | 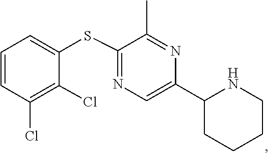 [SMILES: Cc1nc(C2CCCCN2)cnc1Sc1cccc(Cl)c1Cl] |
| 27 | 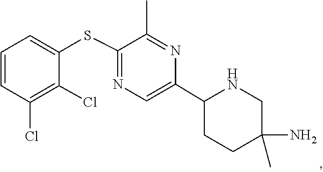 [SMILES: Cc1nc(C2CCC(C)(N)CN2)cnc1Sc1cccc(Cl)c1Cl] |
| 28 | 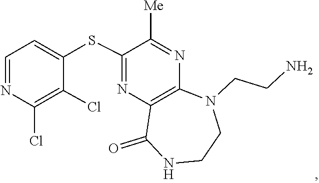 [SMILES: Cc1nc2c(nc1Sc1ccnc(Cl)c1Cl)C(=O)NCCN2CCN] |
| 29 | 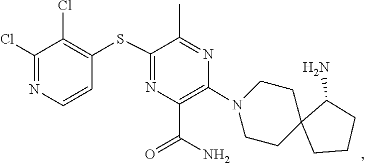 [SMILES: Cc1nc(N2CCC3(CCC[C@H]3N)CC2)c(C(N)=O)nc1Sc1ccnc(Cl)c1Cl] |
| 30 | 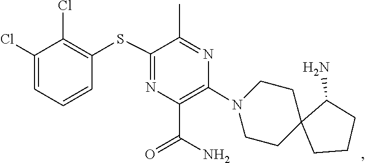 [SMILES: Cc1nc(N2CCC3(CCC[C@H]3N)CC2)c(C(N)=O)nc1Sc1cccc(Cl)c1Cl] |
| 31 | 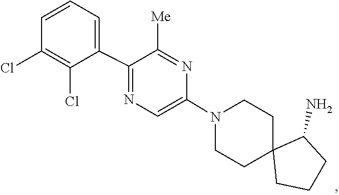 [SMILES: Cc1nc(N2CCC3(CCC[C@H]3N)CC2)cnc1-c1cccc(Cl)c1Cl] |
| 32 | 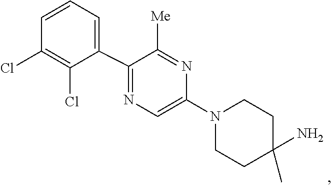 [SMILES: Cc1nc(N2CCC(C)(N)CC2)cnc1-c1cccc(Cl)c1Cl] |
| 33 | 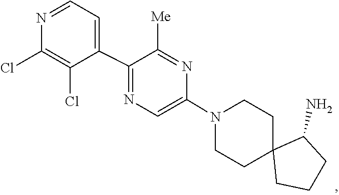 [SMILES: Cc1nc(N2CCC3(CCC[C@H]3N)CC2)cnc1-c1ccnc(Cl)c1Cl] |
| 34 | 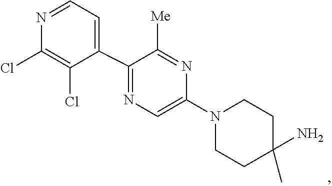 [SMILES: Cc1nc(N2CCC(C)(N)CC2)cnc1-c1ccnc(Cl)c1Cl] |
| 35 | 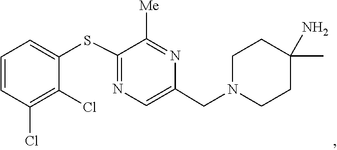 [SMILES: Cc1nc(CN2CCC(C)(N)CC2)cnc1Sc1cccc(Cl)c1Cl] |
| 36 | 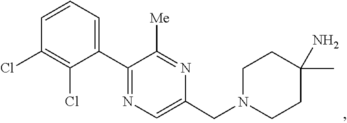 [SMILES: Cc1nc(CN2CCC(C)(N)CC2)cnc1-c1cccc(Cl)c1Cl] |
| 37 | 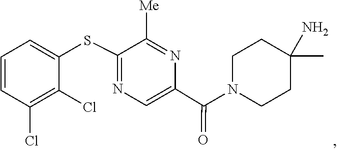 [SMILES: Cc1nc(C(=O)N2CCC(C)(N)CC2)cnc1Sc1cccc(Cl)c1Cl] |
| 38 | 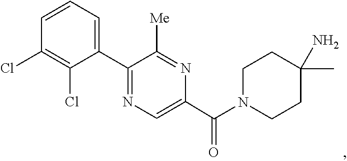 [SMILES: Cc1nc(C(=O)N2CCC(C)(N)CC2)cnc1-c1cccc(Cl)c1Cl] |
| 39 | 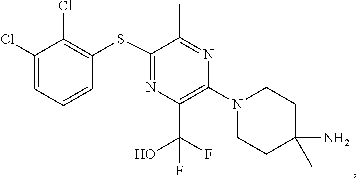 [SMILES: Cc1nc(N2CCC(C)(N)CC2)c(C(O)(F)F)nc1Sc1cccc(Cl)c1Cl] |
| 40 | 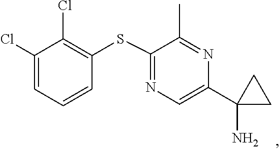 [SMILES: Cc1nc(C2(N)CC2)cnc1Sc1cccc(Cl)c1Cl] |
| 41 | 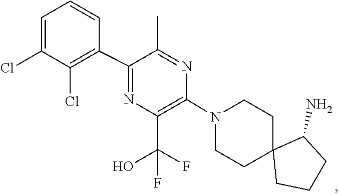 [SMILES: Cc1nc(N2CCC3(CCC[C@H]3N)CC2)c(C(O)(F)F)nc1-c1cccc(Cl)c1Cl] |
| A-1 | 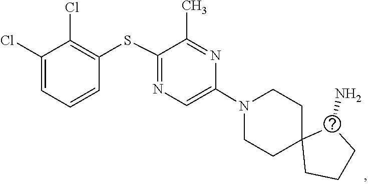 [SMILES: Cc1nc(N2CCC3(CCC[C@H]3N)CC2)cnc1Sc1cccc(Cl)c1Cl] |
| A-2 | 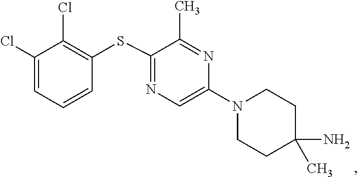 [SMILES: Cc1nc(N2CCC(C)(N)CC2)cnc1Sc1cccc(Cl)c1Cl] |
| A-3 | 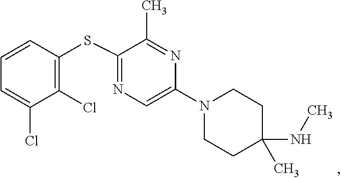 [SMILES: CNC1(C)CCN(c2cnc(Sc3cccc(Cl)c3Cl)c(C)n2)CC1] |
| A-4 | 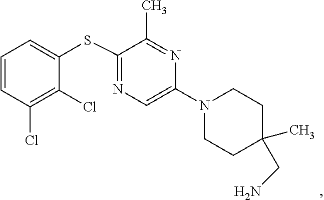 [SMILES: Cc1nc(N2CCC(C)(CN)CC2)cnc1Sc1cccc(Cl)c1Cl] |
| A-5 | 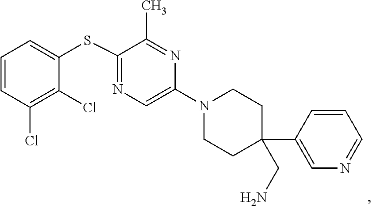 [SMILES: Cc1nc(N2CCC(CN)(c3cccnc3)CC2)cnc1Sc1cccc(Cl)c1Cl] |
| A-6 | 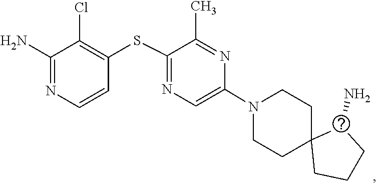 [SMILES: Cc1nc(N2CCC3(CCC[C@H]3N)CC2)cnc1Sc1ccnc(N)c1Cl] |
| A-7 | 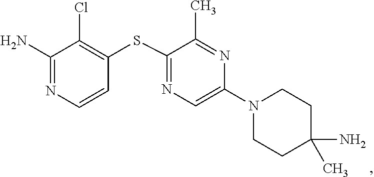 [SMILES: Cc1nc(N2CCC(C)(N)CC2)cnc1Sc1ccnc(N)c1Cl] |
| A-8 | 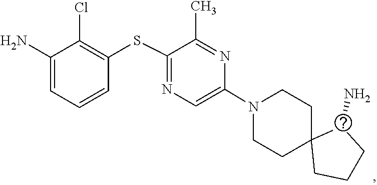 [SMILES: Cc1nc(N2CCC3(CCC[C@H]3N)CC2)cnc1Sc1cccc(N)c1Cl] |
| A-9 | 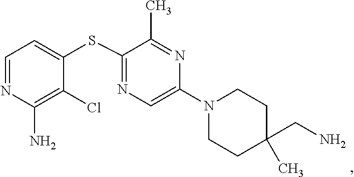 [SMILES: Cc1nc(N2CCC(C)(CN)CC2)cnc1Sc1ccnc(N)c1Cl] |
| A-10 | 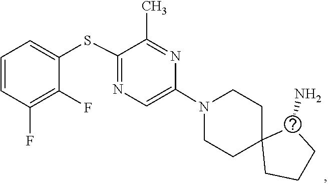 [SMILES: Cc1nc(N2CCC3(CCC[C@H]3N)CC2)cnc1Sc1cccc(F)c1F] |
| A-11 | 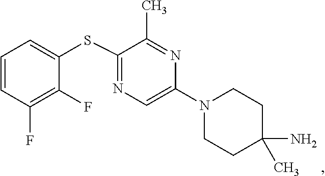 [SMILES: Cc1nc(N2CCC(C)(N)CC2)cnc1Sc1cccc(F)c1F] |
| A-12 | 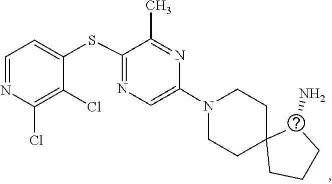 [SMILES: Cc1nc(N2CCC3(CCC[C@H]3N)CC2)cnc1Sc1ccnc(Cl)c1Cl] |
| A-13 | 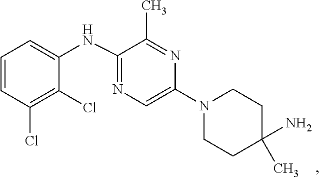 [SMILES: Cc1nc(N2CCC(C)(N)CC2)cnc1Nc1cccc(Cl)c1Cl] |
| A-14 | 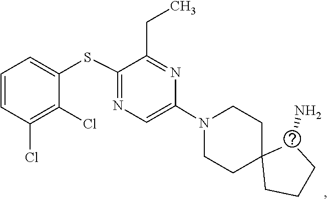 [SMILES: CCc1nc(N2CCC3(CCC[C@H]3N)CC2)cnc1Sc1cccc(Cl)c1Cl] |
| A-15 | 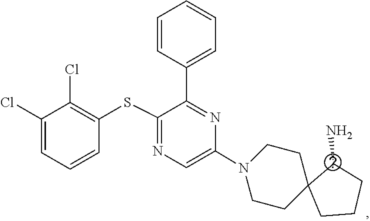 [SMILES: N[C@@H]1CCCC12CCN(c1cnc(Sc3cccc(Cl)c3Cl)c(-c3ccccc3)n1)CC2] |
| A-16 | 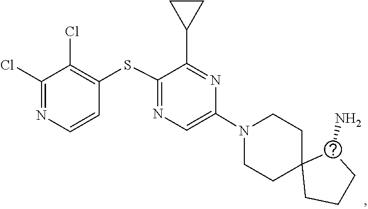 [SMILES: N[C@@H]1CCCC12CCN(c1cnc(Sc3ccnc(Cl)c3Cl)c(C3CC3)n1)CC2] |
| A-17 | 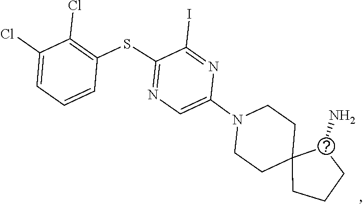 [SMILES: N[C@@H]1CCCC12CCN(c1cnc(Sc3cccc(Cl)c3Cl)c(I)n1)CC2] |
| A-18 | 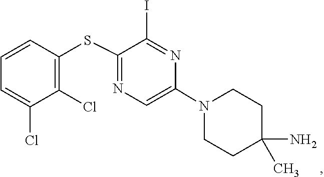 [SMILES: CC1(N)CCN(c2cnc(Sc3cccc(Cl)c3Cl)c(I)n2)CC1] |
| A-19 | 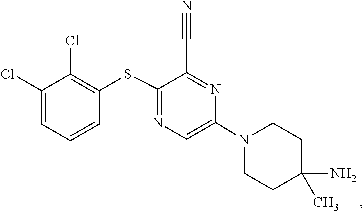 [SMILES: CC1(N)CCN(c2cnc(Sc3cccc(Cl)c3Cl)c(C#N)n2)CC1] |
| A-20 | 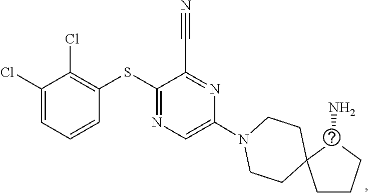 [SMILES: N#Cc1nc(N2CCC3(CCC[C@H]3N)CC2)cnc1Sc1cccc(Cl)c1Cl] |
| A-21 | 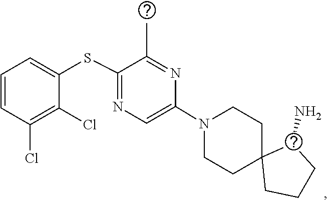 [SMILES: Cc1nc(N2CCC3(CCC[C@H]3N)CC2)cnc1Sc1cccc(Cl)c1Cl] |
| A-22 | 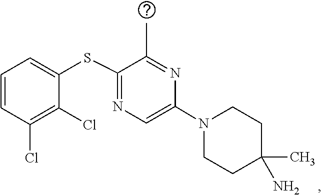 [SMILES: Cc1nc(N2CCC(C)(N)CC2)cnc1Sc1cccc(Cl)c1Cl] |
| A-23 | 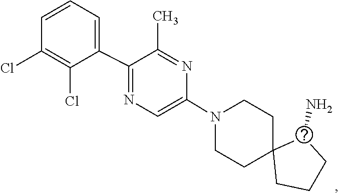 [SMILES: Cc1nc(N2CCC3(CCC[C@H]3N)CC2)cnc1-c1cccc(Cl)c1Cl] |
| A-24 | 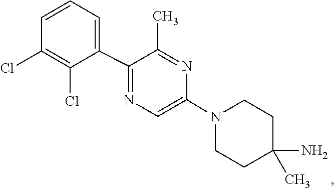 [SMILES: Cc1nc(N2CCC(C)(N)CC2)cnc1-c1cccc(Cl)c1Cl] |
| A-25 | 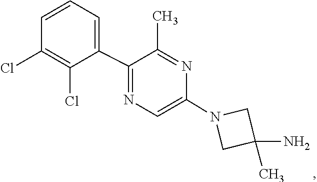 [SMILES: Cc1nc(N2CC(C)(N)C2)cnc1-c1cccc(Cl)c1Cl] |
| A-26 | 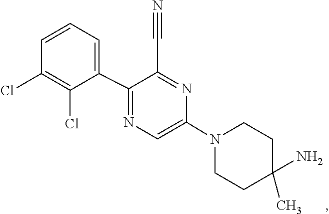 [SMILES: CC1(N)CCN(c2cnc(-c3cccc(Cl)c3Cl)c(C#N)n2)CC1] |
| A-27 | 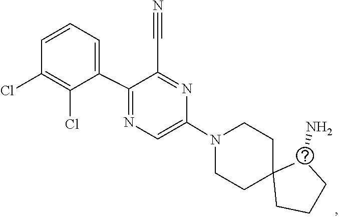 [SMILES: N#Cc1nc(N2CCC3(CCC[C@H]3N)CC2)cnc1-c1cccc(Cl)c1Cl] |
| A-28 | 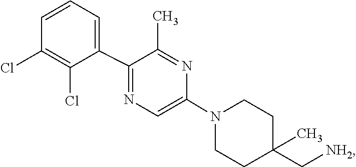 [SMILES: Cc1nc(N2CCC(C)(CN)CC2)cnc1-c1cccc(Cl)c1Cl] |
| A-29 | 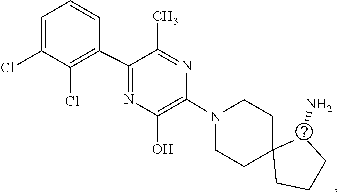 [SMILES: Cc1nc(N2CCC3(CCC[C@H]3N)CC2)c(O)nc1-c1cccc(Cl)c1Cl] |
| A-30 | 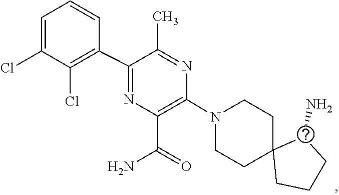 [SMILES: Cc1nc(N2CCC3(CCC[C@H]3N)CC2)c(C(N)=O)nc1-c1cccc(Cl)c1Cl] |
| A-31 | 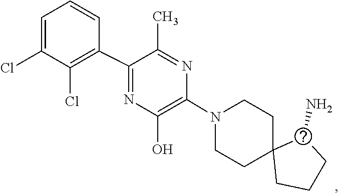 [SMILES: Cc1nc(N2CCC3(CCC[C@H]3N)CC2)c(O)nc1-c1cccc(Cl)c1Cl] |
| A-32 | 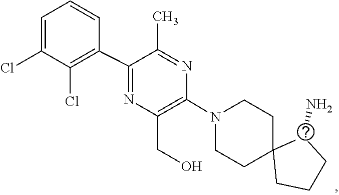 [SMILES: Cc1nc(N2CCC3(CCC[C@H]3N)CC2)c(CO)nc1-c1cccc(Cl)c1Cl] |
| A-33 | 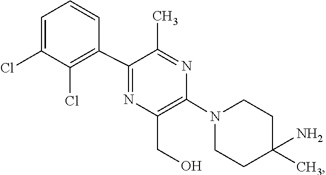 [SMILES: Cc1nc(N2CCC(C)(N)CC2)c(CO)nc1-c1cccc(Cl)c1Cl] |
| A-34 | 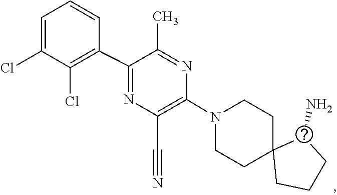 [SMILES: Cc1nc(N2CCC3(CCC[C@H]3N)CC2)c(C#N)nc1-c1cccc(Cl)c1Cl] |
| A-35 | 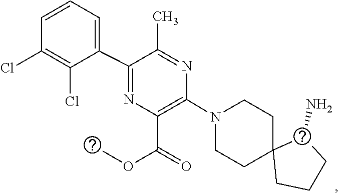 [SMILES: COC(=O)c1nc(-c2cccc(Cl)c2Cl)c(C)nc1N1CCC2(CCC[C@H]2N)CC1] |
| A-36 | 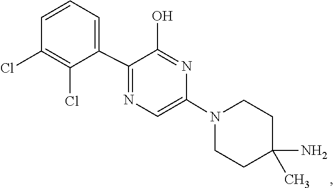 [SMILES: CC1(N)CCN(c2cnc(-c3cccc(Cl)c3Cl)c(O)n2)CC1] |
| A-37 | 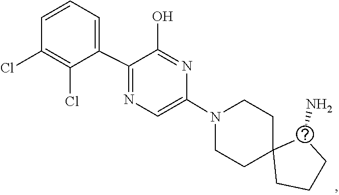 [SMILES: N[C@@H]1CCCC12CCN(c1cnc(-c3cccc(Cl)c3Cl)c(O)n1)CC2] |
| A-38 | 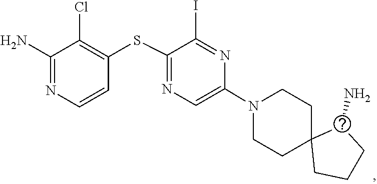 [SMILES: Nc1nccc(Sc2ncc(N3CCC4(CCC[C@H]4N)CC3)nc2I)c1Cl] |
| A-39 | 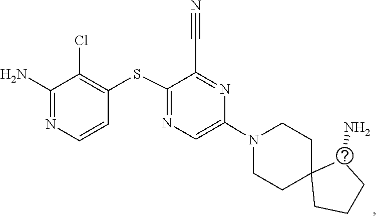 [SMILES: N#Cc1nc(N2CCC3(CCC[C@H]3N)CC2)cnc1Sc1ccnc(N)c1Cl] |
| A-40 | 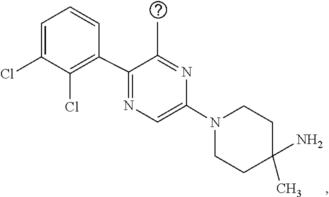 [SMILES: Cc1nc(N2CCC(C)(N)CC2)cnc1-c1cccc(Cl)c1Cl] |
| A-41 | 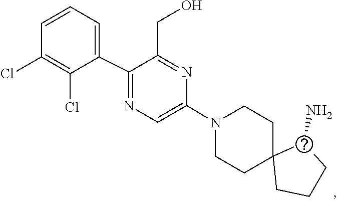 [SMILES: N[C@@H]1CCCC12CCN(c1cnc(-c3cccc(Cl)c3Cl)c(CO)n1)CC2] |
| A-42 | 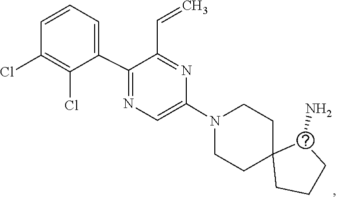 [SMILES: C=Cc1nc(N2CCC3(CCC[C@H]3N)CC2)cnc1-c1cccc(Cl)c1Cl] |
| A-43 | 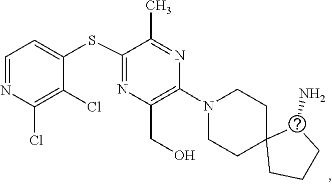 [SMILES: Cc1nc(N2CCC3(CCC[C@H]3N)CC2)c(CO)nc1Sc1ccnc(Cl)c1Cl] |
| A-44 | 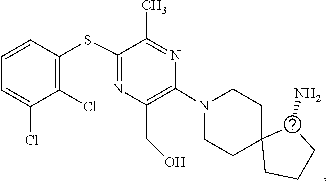 [SMILES: Cc1nc(N2CCC3(CCC[C@H]3N)CC2)c(CO)nc1Sc1cccc(Cl)c1Cl] |
| A-45 | 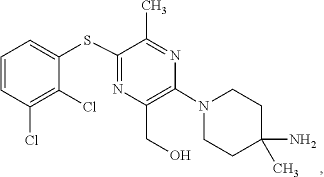 [SMILES: Cc1nc(N2CCC(C)(N)CC2)c(CO)nc1Sc1cccc(Cl)c1Cl] |
| A-46 | 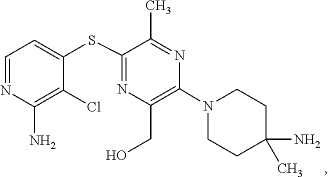 [SMILES: Cc1nc(N2CCC(C)(N)CC2)c(CO)nc1Sc1ccnc(N)c1Cl] |
| A-47 | 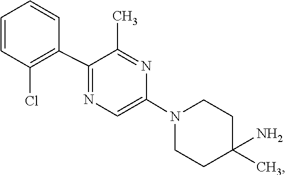 [SMILES: Cc1nc(N2CCC(C)(N)CC2)cnc1-c1ccccc1Cl] |
| A-48 | 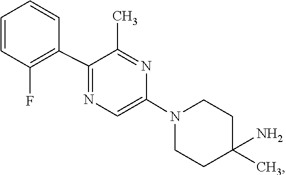 [SMILES: Cc1nc(N2CCC(C)(N)CC2)cnc1-c1ccccc1F] |
| A-49 | 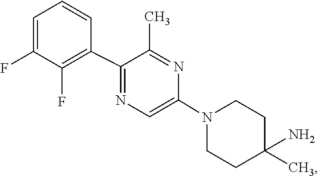 [SMILES: Cc1nc(N2CCC(C)(N)CC2)cnc1-c1cccc(F)c1F] |
| A-50 | 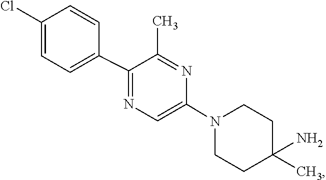 [SMILES: Cc1nc(N2CCC(C)(N)CC2)cnc1-c1ccc(Cl)cc1] |
| A-51 | 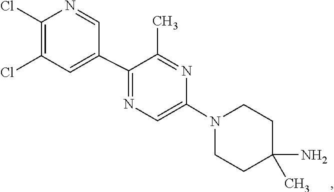 [SMILES: Cc1nc(N2CCC(C)(N)CC2)cnc1-c1cnc(Cl)c(Cl)c1] |
| A-52 | 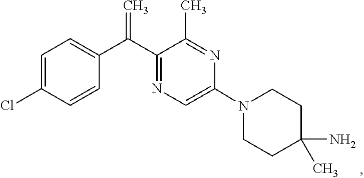 [SMILES: C=C(c1ccc(Cl)cc1)c1ncc(N2CCC(C)(N)CC2)nc1C] |
| A-53 | 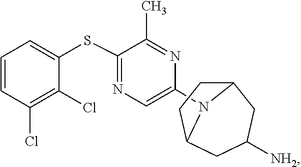 [SMILES: Cc1nc(N2C3CCC2CC(N)C3)cnc1Sc1cccc(Cl)c1Cl] |
| A-54 | 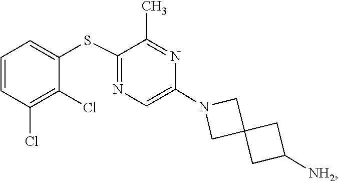 [SMILES: Cc1nc(N2CC3(CC(N)C3)C2)cnc1Sc1cccc(Cl)c1Cl] |
| A-55 | 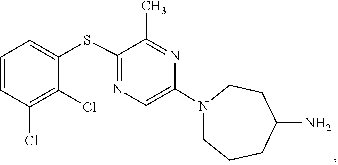 [SMILES: Cc1nc(N2CCCC(N)CC2)cnc1Sc1cccc(Cl)c1Cl] |
| A-56 | 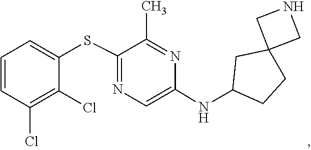 [SMILES: Cc1nc(NC2CCC3(CNC3)C2)cnc1Sc1cccc(Cl)c1Cl] |
| A-57 | 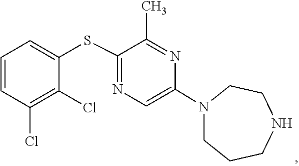 [SMILES: Cc1nc(N2CCCNCC2)cnc1Sc1cccc(Cl)c1Cl] |
| A-58 | 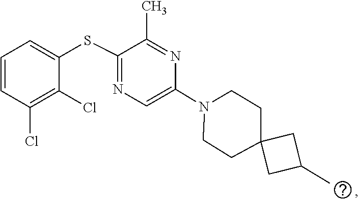 [SMILES: Cc1nc(N2CCC3(CC2)CC(C)C3)cnc1Sc1cccc(Cl)c1Cl] |
| A-59 | 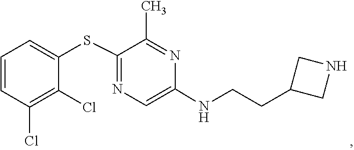 [SMILES: Cc1nc(NCCC2CNC2)cnc1Sc1cccc(Cl)c1Cl] |
| A-60 | 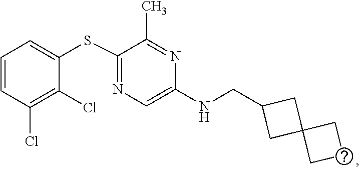 [SMILES: Cc1nc(NCC2CC3(CCC3)C2)cnc1Sc1cccc(Cl)c1Cl] |
| A-61 | 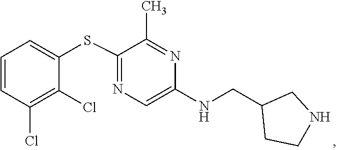 [SMILES: Cc1nc(NCC2CCNC2)cnc1Sc1cccc(Cl)c1Cl] |
| A-62 | 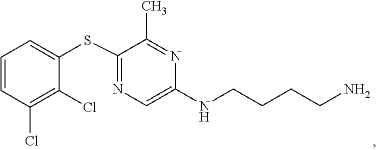 [SMILES: Cc1nc(NCCCCN)cnc1Sc1cccc(Cl)c1Cl] |
| A-63 | 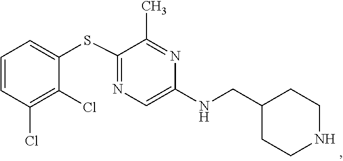 [SMILES: Cc1nc(NCC2CCNCC2)cnc1Sc1cccc(Cl)c1Cl] |
| A-64 | 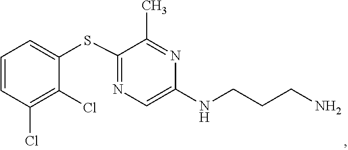 [SMILES: Cc1nc(NCCCN)cnc1Sc1cccc(Cl)c1Cl] |
| A-65 | 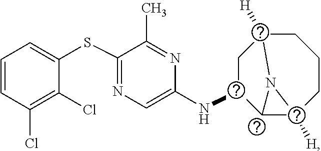 |
| A-66 | 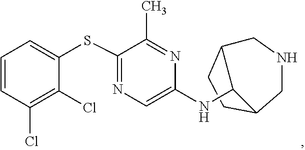 [SMILES: Cc1nc(NC2C3CCC2CNC3)cnc1Sc1cccc(Cl)c1Cl] |
| A-67 | 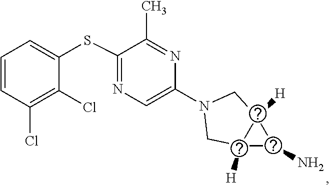 [SMILES: Cc1nc(N2C[C@@H]3[C@@H](N)[C@@H]3C2)cnc1Sc1cccc(Cl)c1Cl] |
| A-68 | 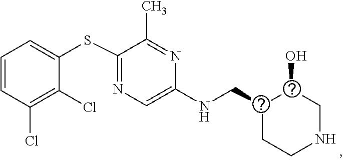 [SMILES: Cc1nc(NC[C@@H]2CCNC[C@@H]2O)cnc1Sc1cccc(Cl)c1Cl] |
| A-69 | 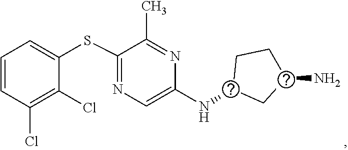 [SMILES: Cc1nc(N[C@@H]2CC[C@@H](N)C2)cnc1Sc1cccc(Cl)c1Cl] |
| A-70 | 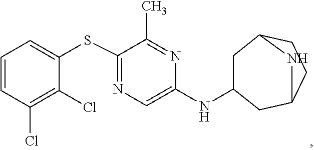 [SMILES: Cc1nc(NC2CC3CCC(C2)N3)cnc1Sc1cccc(Cl)c1Cl] |
| A-71 | 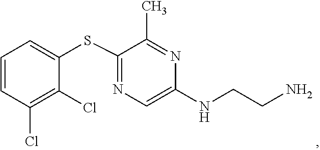 [SMILES: Cc1nc(NCCN)cnc1Sc1cccc(Cl)c1Cl] |
| A-72 | 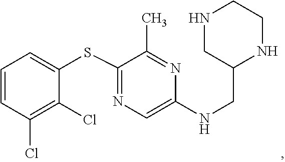 [SMILES: Cc1nc(NCC2CNCCN2)cnc1Sc1cccc(Cl)c1Cl] |
| A-73 | 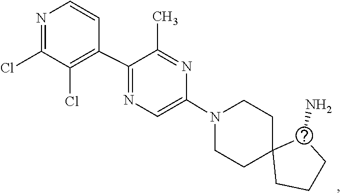 [SMILES: Cc1nc(N2CCC3(CCC[C@H]3N)CC2)cnc1-c1ccnc(Cl)c1Cl] |
| A-74 | 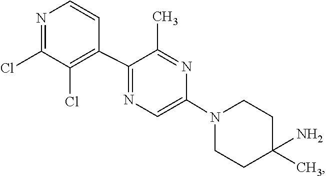 [SMILES: Cc1nc(N2CCC(C)(N)CC2)cnc1-c1ccnc(Cl)c1Cl] |
| A-75 | 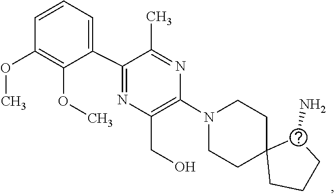 [SMILES: COc1cccc(-c2nc(CO)c(N3CCC4(CCC[C@H]4N)CC3)nc2C)c1OC] |
| A-76 | 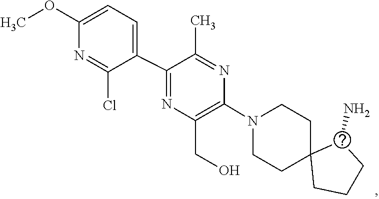 [SMILES: COc1ccc(-c2nc(CO)c(N3CCC4(CCC[C@H]4N)CC3)nc2C)c(Cl)n1] |
| A-77 | 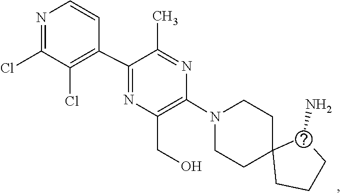 [SMILES: Cc1nc(N2CCC3(CCC[C@H]3N)CC2)c(CO)nc1-c1ccnc(Cl)c1Cl] |
| A-78 | 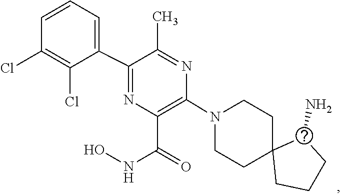 [SMILES: Cc1nc(N2CCC3(CCC[C@H]3N)CC2)c(C(=O)NO)nc1-c1cccc(Cl)c1Cl] |
| A-79 | 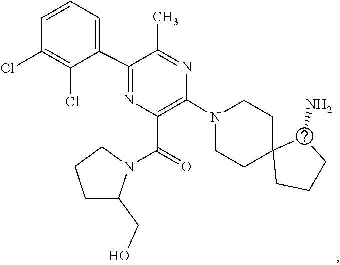 [SMILES: Cc1nc(N2CCC3(CCC[C@H]3N)CC2)c(C(=O)N2CCCC2CO)nc1-c1cccc(Cl)c1Cl] |
| A-80 | 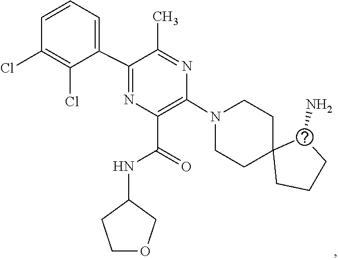 [SMILES: Cc1nc(N2CCC3(CCC[C@H]3N)CC2)c(C(=O)NC2CCOC2)nc1-c1cccc(Cl)c1Cl] |
| A-81 | 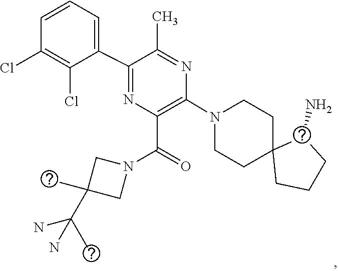 [SMILES: Cc1nc(N2CCC3(CCC[C@H]3N)CC2)c(C(=O)N2CC(C)(C(C)(N)N)C2)nc1-c1cccc(Cl)c1Cl] |
| A-82 | 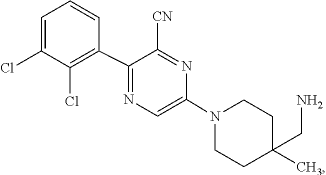 [SMILES: CC1(CN)CCN(c2cnc(-c3cccc(Cl)c3Cl)c(C#N)n2)CC1] |
| A-83 | 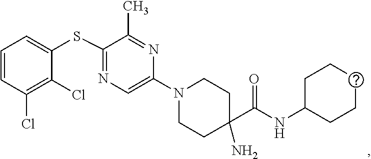 [SMILES: Cc1nc(N2CCC(N)(C(=O)NC3CCCCC3)CC2)cnc1Sc1cccc(Cl)c1Cl] |
| A-84 | 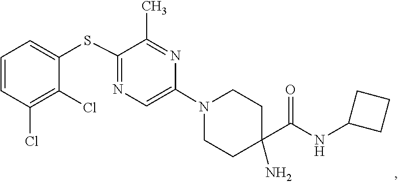 [SMILES: Cc1nc(N2CCC(N)(C(=O)NC3CCC3)CC2)cnc1Sc1cccc(Cl)c1Cl] |
| A-85 | 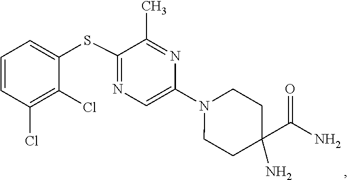 [SMILES: Cc1nc(N2CCC(N)(C(N)=O)CC2)cnc1Sc1cccc(Cl)c1Cl] |
| A-86 | 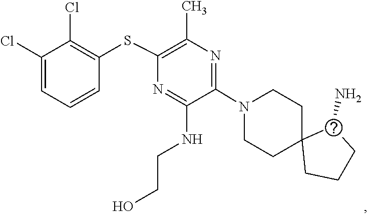 [SMILES: Cc1nc(N2CCC3(CCC[C@H]3N)CC2)c(NCCO)nc1Sc1cccc(Cl)c1Cl] |
| A-87 | 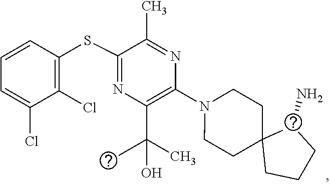 [SMILES: Cc1nc(N2CCC3(CCC[C@H]3N)CC2)c(C(C)(C)O)nc1Sc1cccc(Cl)c1Cl] |
| A-88 | 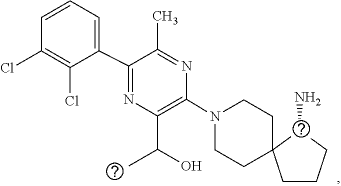 [SMILES: Cc1nc(N2CCC3(CCC[C@H]3N)CC2)c(C(C)O)nc1-c1cccc(Cl)c1Cl] |
| A-89 | 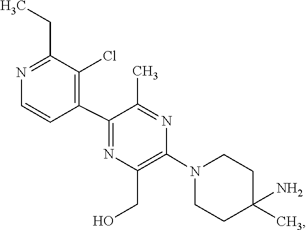 [SMILES: CCc1nccc(-c2nc(CO)c(N3CCC(C)(N)CC3)nc2C)c1Cl] |
| A-90 | 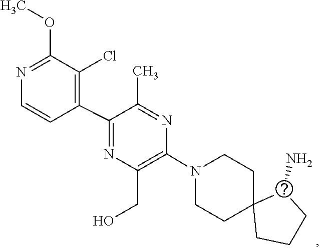 [SMILES: COc1nccc(-c2nc(CO)c(N3CCC4(CCC[C@H]4N)CC3)nc2C)c1Cl] |
| A-91 | 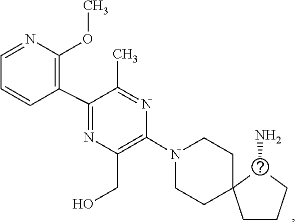 [SMILES: COc1ncccc1-c1nc(CO)c(N2CCC3(CCC[C@H]3N)CC2)nc1C] |
| A-92 | 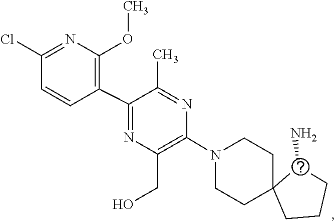 [SMILES: COc1nc(Cl)ccc1-c1nc(CO)c(N2CCC3(CCC[C@H]3N)CC2)nc1C] |
| A-93 | 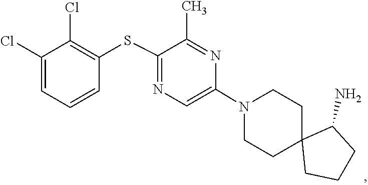 [SMILES: Cc1nc(N2CCC3(CCC[C@H]3N)CC2)cnc1Sc1cccc(Cl)c1Cl] |
| A-94 | 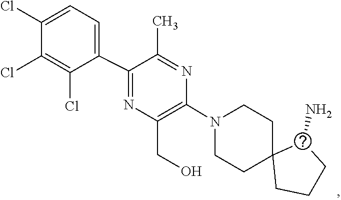 [SMILES: Cc1nc(N2CCC3(CCC[C@H]3N)CC2)c(CO)nc1-c1ccc(Cl)c(Cl)c1Cl] |
| A-95 | 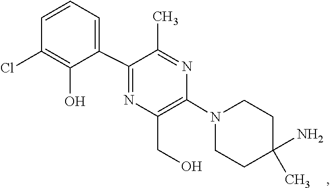 [SMILES: Cc1nc(N2CCC(C)(N)CC2)c(CO)nc1-c1cccc(Cl)c1O] |
| A-96 | 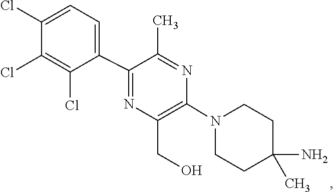 [SMILES: Cc1nc(N2CCC(C)(N)CC2)c(CO)nc1-c1ccc(Cl)c(Cl)c1Cl] |
| A-97 | 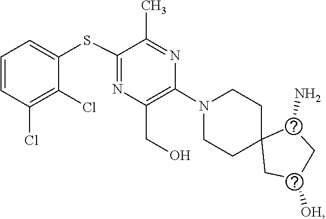 [SMILES: Cc1nc(N2CCC3(CC2)C[C@@H](O)C[C@H]3N)c(CO)nc1Sc1cccc(Cl)c1Cl] |
| A-98 | 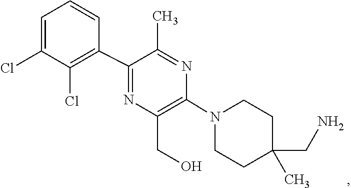 [SMILES: Cc1nc(N2CCC(C)(CN)CC2)c(CO)nc1-c1cccc(Cl)c1Cl] |
| A-99 | 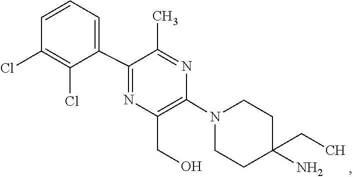 [SMILES: CCC1(N)CCN(c2nc(C)c(-c3cccc(Cl)c3Cl)nc2CO)CC1] |
| A-100 | 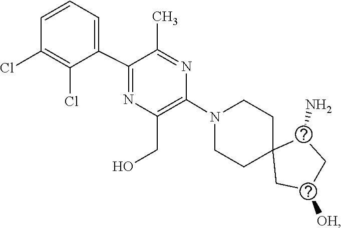 [SMILES: Cc1nc(N2CCC3(CC2)C[C@H](O)C[C@H]3N)c(CO)nc1-c1cccc(Cl)c1Cl] |
| A-101 | 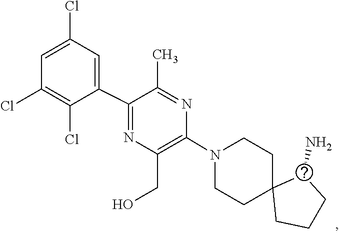 [SMILES: Cc1nc(N2CCC3(CCC[C@H]3N)CC2)c(CO)nc1-c1cc(Cl)cc(Cl)c1Cl] |
| A-102 | 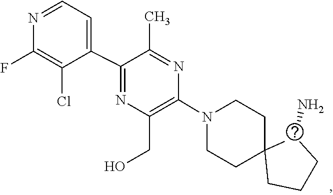 [SMILES: Cc1nc(N2CCC3(CCC[C@H]3N)CC2)c(CO)nc1-c1ccnc(F)c1Cl] |
| A-103 | 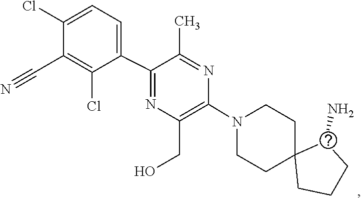 [SMILES: Cc1nc(N2CCC3(CCC[C@H]3N)CC2)c(CO)nc1-c1ccc(Cl)c(C#N)c1Cl] |
| A-104 | 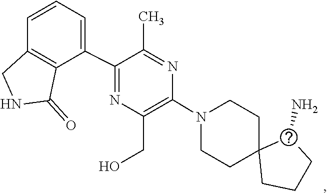 [SMILES: Cc1nc(N2CCC3(CCC[C@H]3N)CC2)c(CO)nc1-c1cccc2c1C(=O)NC2] |
| A-105 | 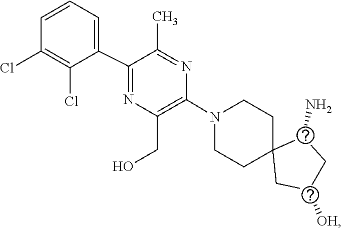 [SMILES: Cc1nc(N2CCC3(CC2)C[C@@H](O)C[C@H]3N)c(CO)nc1-c1cccc(Cl)c1Cl] |
| A-106 | 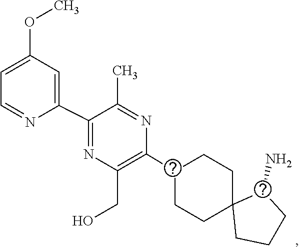 [SMILES: COc1ccnc(-c2nc(CO)c(C3CCC4(CCC[C@H]4N)CC3)nc2C)c1] |
| A-107 | 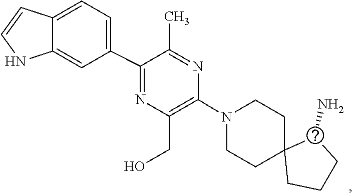 [SMILES: Cc1nc(N2CCC3(CCC[C@H]3N)CC2)c(CO)nc1-c1ccc2cc[nH]c2c1] |
| A-108 | 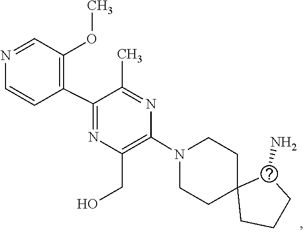 [SMILES: COc1cnccc1-c1nc(CO)c(N2CCC3(CCC[C@H]3N)CC2)nc1C] |
| A-109 | 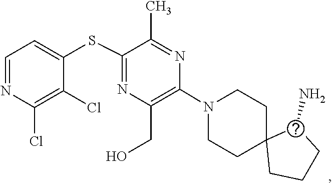 [SMILES: Cc1nc(N2CCC3(CCC[C@H]3N)CC2)c(CO)nc1Sc1ccnc(Cl)c1Cl] |
| A-110 | 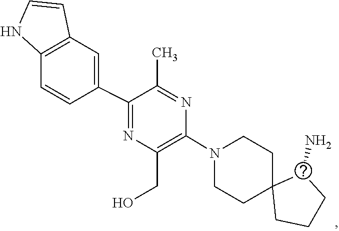 [SMILES: Cc1nc(N2CCC3(CCC[C@H]3N)CC2)c(CO)nc1-c1ccc2[nH]ccc2c1] |
| A-111 | 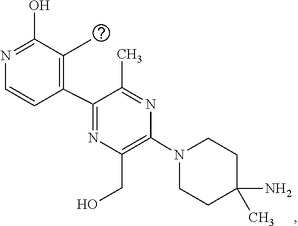 [SMILES: Cc1nc(N2CCC(C)(N)CC2)c(CO)nc1-c1ccnc(O)c1C] |
| A-112 | 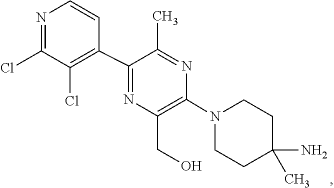 [SMILES: Cc1nc(N2CCC(C)(N)CC2)c(CO)nc1-c1ccnc(Cl)c1Cl] |
| A-113 | 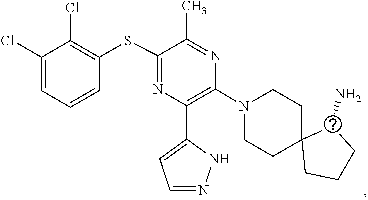 [SMILES: Cc1nc(N2CCC3(CCC[C@H]3N)CC2)c(-c2ccn[nH]2)nc1Sc1cccc(Cl)c1Cl] |
| A-114 | 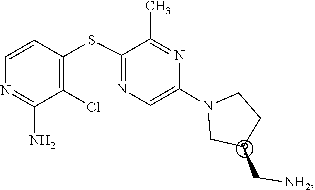 [SMILES: Cc1nc(N2CC[C@@H](CN)C2)cnc1Sc1ccnc(N)c1Cl] |
| A-115 | 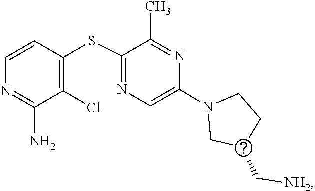 [SMILES: Cc1nc(N2CC[C@H](CN)C2)cnc1Sc1ccnc(N)c1Cl] |
| A-116 | 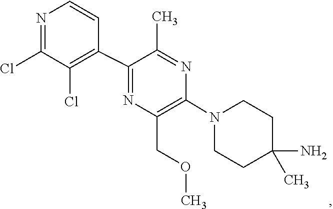 [SMILES: COCc1nc(-c2ccnc(Cl)c2Cl)c(C)nc1N1CCC(C)(N)CC1] |
| A-117 | 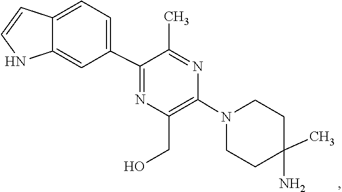 [SMILES: Cc1nc(N2CCC(C)(N)CC2)c(CO)nc1-c1ccc2cc[nH]c2c1] |
| A-118 | 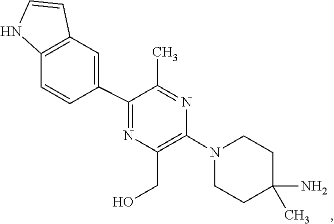 [SMILES: Cc1nc(N2CCC(C)(N)CC2)c(CO)nc1-c1ccc2[nH]ccc2c1] |
| A-119 | 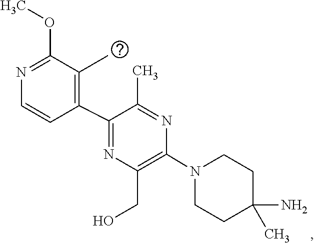 [SMILES: COc1nccc(-c2nc(CO)c(N3CCC(C)(N)CC3)nc2C)c1C] |
| A-120 | 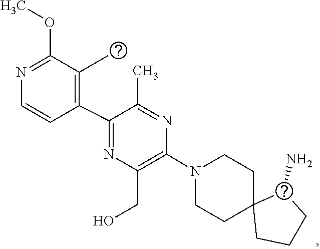 [SMILES: COc1nccc(-c2nc(CO)c(N3CCC4(CCC[C@H]4N)CC3)nc2C)c1C] |
| A-121 | 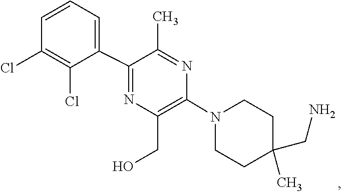 [SMILES: Cc1nc(N2CCC(C)(CN)CC2)c(CO)nc1-c1cccc(Cl)c1Cl] |
| A-122 | 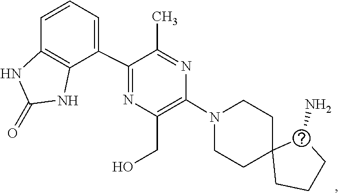 [SMILES: Cc1nc(N2CCC3(CCC[C@H]3N)CC2)c(CO)nc1-c1cccc2[nH]c(=O)[nH]c12] |
| A-123 | 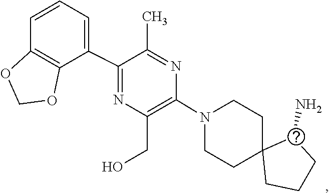 [SMILES: Cc1nc(N2CCC3(CCC[C@H]3N)CC2)c(CO)nc1-c1cccc2c1OCO2] |
| A-124 | 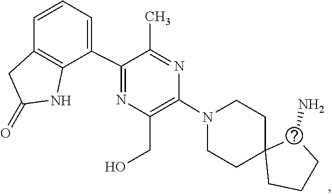 [SMILES: Cc1nc(N2CCC3(CCC[C@H]3N)CC2)c(CO)nc1-c1cccc2c1NC(=O)C2] |
| A-125 | 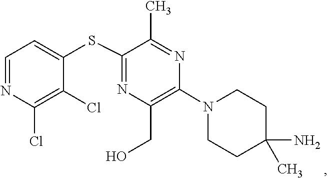 [SMILES: Cc1nc(N2CCC(C)(N)CC2)c(CO)nc1Sc1ccnc(Cl)c1Cl] |
| A-126 | 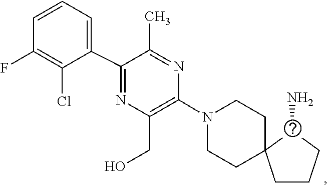 [SMILES: Cc1nc(N2CCC3(CCC[C@H]3N)CC2)c(CO)nc1-c1cccc(F)c1Cl] |
| A-127 | 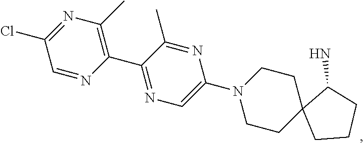 [SMILES: Cc1nc(Cl)cnc1-c1ncc(N2CCC3(CCC[C@H]3N)CC2)nc1C] |
| A-128 | 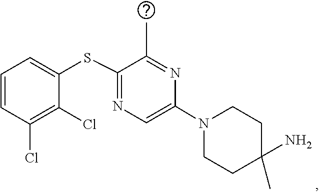 [SMILES: Cc1nc(N2CCC(C)(N)CC2)cnc1Sc1cccc(Cl)c1Cl] |
| A-129 | 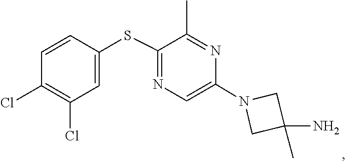 [SMILES: Cc1nc(N2CC(C)(N)C2)cnc1Sc1ccc(Cl)c(Cl)c1] |
| A-130 | 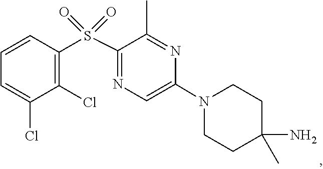 [SMILES: Cc1nc(N2CCC(C)(N)CC2)cnc1S(=O)(=O)c1cccc(Cl)c1Cl] |
| A-131 | 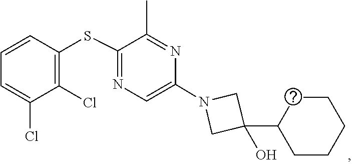 [SMILES: Cc1nc(N2CC(O)(C3CCCCC3)C2)cnc1Sc1cccc(Cl)c1Cl] |
| A-132 | 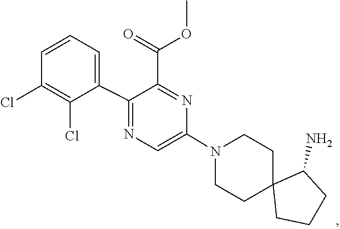 [SMILES: COC(=O)c1nc(N2CCC3(CCC[C@H]3N)CC2)cnc1-c1cccc(Cl)c1Cl] |
| A-133 | 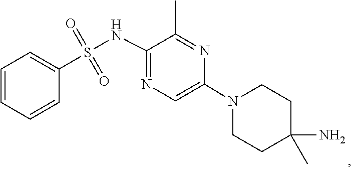 [SMILES: Cc1nc(N2CCC(C)(N)CC2)cnc1NS(=O)(=O)c1ccccc1] |
| A-134 | 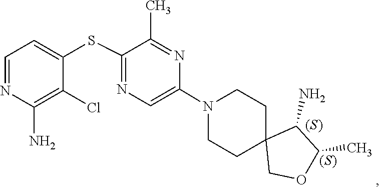 |
| A-135 | 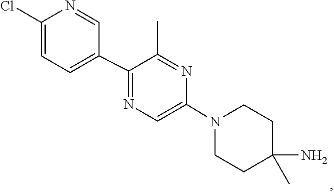 [SMILES: Cc1nc(N2CCC(C)(N)CC2)cnc1-c1ccc(Cl)nc1] |
| A-136 | 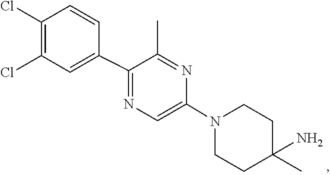 [SMILES: Cc1nc(N2CCC(C)(N)CC2)cnc1-c1ccc(Cl)c(Cl)c1] |
| A-137 | 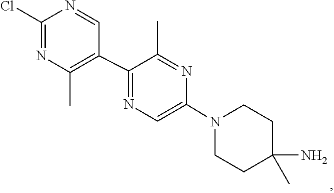 [SMILES: Cc1nc(Cl)ncc1-c1ncc(N2CCC(C)(N)CC2)nc1C] |
| A-138 | 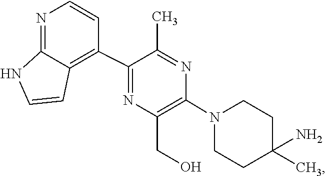 [SMILES: Cc1nc(N2CCC(C)(N)CC2)c(CO)nc1-c1ccnc2[nH]ccc12] |
| A-139 | 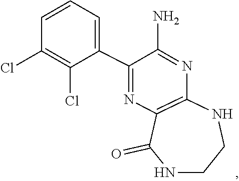 [SMILES: Nc1nc2c(nc1-c1cccc(Cl)c1Cl)C(=O)NCCN2] |
| A-140 | 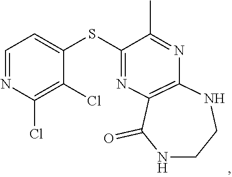 [SMILES: Cc1nc2c(nc1Sc1ccnc(Cl)c1Cl)C(=O)NCCN2] |
| A-141 | 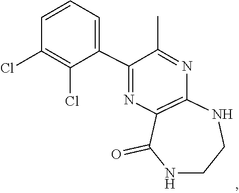 [SMILES: Cc1nc2c(nc1-c1cccc(Cl)c1Cl)C(=O)NCCN2] |
| A-142 | 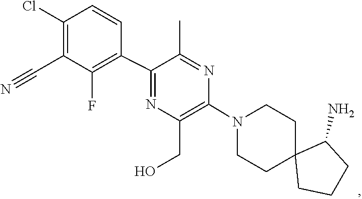 [SMILES: Cc1nc(N2CCC3(CCC[C@H]3N)CC2)c(CO)nc1-c1ccc(Cl)c(C#N)c1F] |
| A-143 | 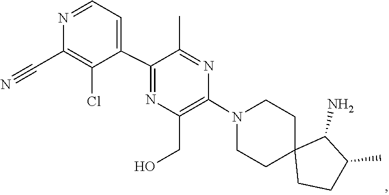 [SMILES: Cc1nc(N2CCC3(CC[C@@H](C)[C@H]3N)CC2)c(CO)nc1-c1ccnc(C#N)c1Cl] |
| A-144 | 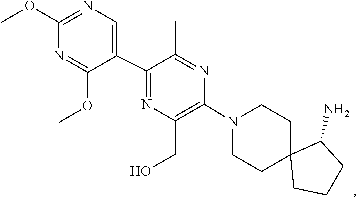 [SMILES: COc1ncc(-c2nc(CO)c(N3CCC4(CCC[C@H]4N)CC3)nc2C)c(OC)n1] |
| A-145 | 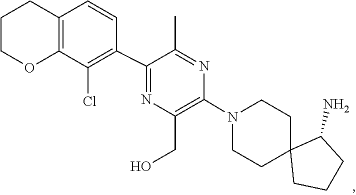 [SMILES: Cc1nc(N2CCC3(CCC[C@H]3N)CC2)c(CO)nc1-c1ccc2c(c1Cl)OCCC2] |
| A-146 | 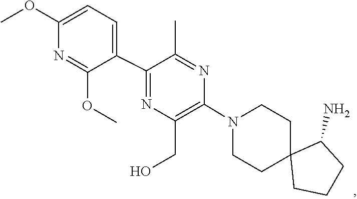 [SMILES: COc1ccc(-c2nc(CO)c(N3CCC4(CCC[C@H]4N)CC3)nc2C)c(OC)n1] |
| A-147 | 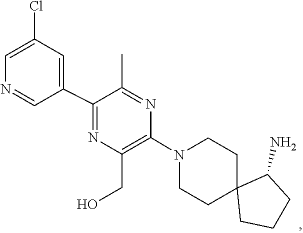 [SMILES: Cc1nc(N2CCC3(CCC[C@H]3N)CC2)c(CO)nc1-c1cncc(Cl)c1] |
| A-148 | 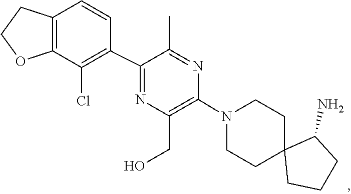 [SMILES: Cc1nc(N2CCC3(CCC[C@H]3N)CC2)c(CO)nc1-c1ccc2c(c1Cl)OCC2] |
| A-149 | 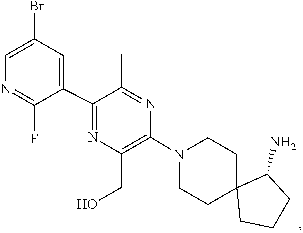 [SMILES: Cc1nc(N2CCC3(CCC[C@H]3N)CC2)c(CO)nc1-c1cc(Br)cnc1F] |
| A-150 | 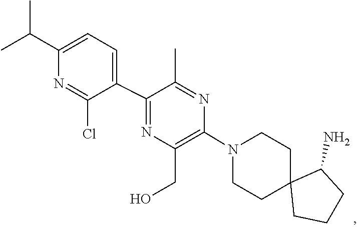 [SMILES: Cc1nc(N2CCC3(CCC[C@H]3N)CC2)c(CO)nc1-c1ccc(C(C)C)nc1Cl] |
| A-151 | 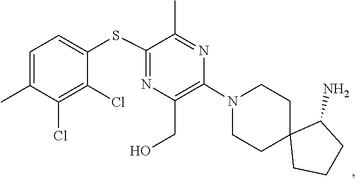 [SMILES: Cc1ccc(Sc2nc(CO)c(N3CCC4(CCC[C@H]4N)CC3)nc2C)c(Cl)c1Cl] |
| A-152 | 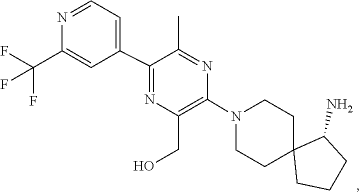 [SMILES: Cc1nc(N2CCC3(CCC[C@H]3N)CC2)c(CO)nc1-c1ccnc(C(F)(F)F)c1] |
| A-153 | 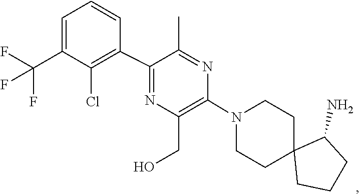 [SMILES: Cc1nc(N2CCC3(CCC[C@H]3N)CC2)c(CO)nc1-c1cccc(C(F)(F)F)c1Cl] |
| A-154 | 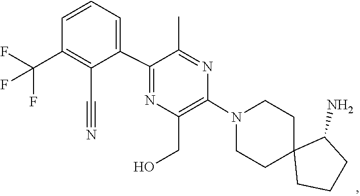 [SMILES: Cc1nc(N2CCC3(CCC[C@H]3N)CC2)c(CO)nc1-c1cccc(C(F)(F)F)c1C#N] |
| A-155 | 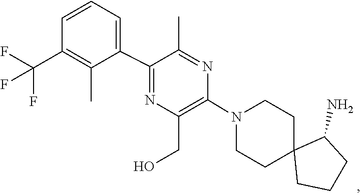 [SMILES: Cc1nc(N2CCC3(CCC[C@H]3N)CC2)c(CO)nc1-c1cccc(C(F)(F)F)c1C] |
| A-156 | 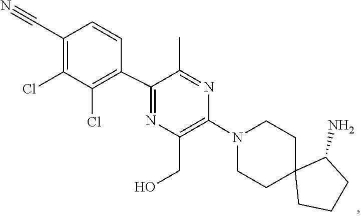 [SMILES: Cc1nc(N2CCC3(CCC[C@H]3N)CC2)c(CO)nc1-c1ccc(C#N)c(Cl)c1Cl] |
| A-157 | 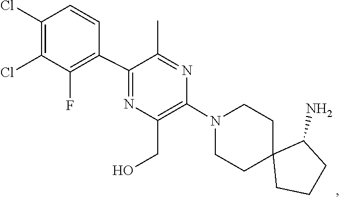 [SMILES: Cc1nc(N2CCC3(CCC[C@H]3N)CC2)c(CO)nc1-c1ccc(Cl)c(Cl)c1F] |
| A-158 | 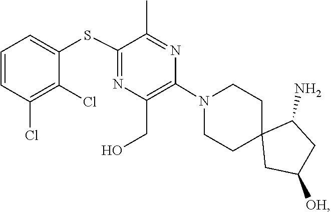 [SMILES: Cc1nc(N2CCC3(CC2)C[C@H](O)C[C@H]3N)c(CO)nc1Sc1cccc(Cl)c1Cl] |
| A-159 | 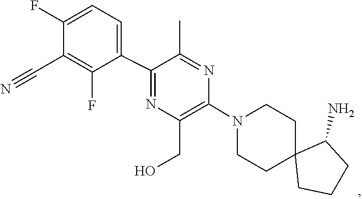 [SMILES: Cc1nc(N2CCC3(CCC[C@H]3N)CC2)c(CO)nc1-c1ccc(F)c(C#N)c1F] |
| A-160 | 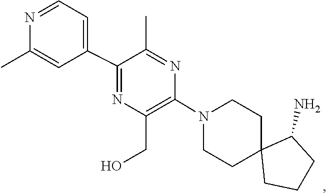 [SMILES: Cc1cc(-c2nc(CO)c(N3CCC4(CCC[C@H]4N)CC3)nc2C)ccn1] |
| A-161 | 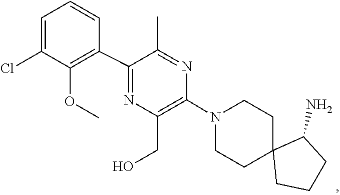 [SMILES: COc1c(Cl)cccc1-c1nc(CO)c(N2CCC3(CCC[C@H]3N)CC2)nc1C] |
| A-162 | 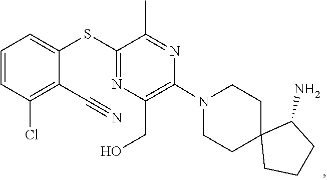 [SMILES: Cc1nc(N2CCC3(CCC[C@H]3N)CC2)c(CO)nc1Sc1cccc(Cl)c1C#N] |
| A-163 | 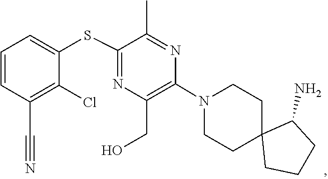 [SMILES: Cc1nc(N2CCC3(CCC[C@H]3N)CC2)c(CO)nc1Sc1cccc(C#N)c1Cl] |
| A-164 | 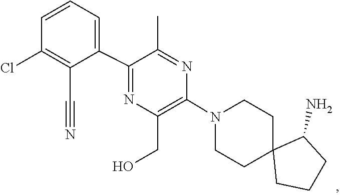 [SMILES: Cc1nc(N2CCC3(CCC[C@H]3N)CC2)c(CO)nc1-c1cccc(Cl)c1C#N] |
| A-165 | 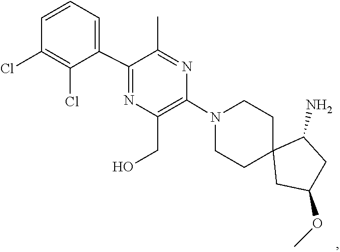 [SMILES: CO[C@@H]1C[C@@H](N)C2(CCN(c3nc(C)c(-c4cccc(Cl)c4Cl)nc3CO)CC2)C1] |
| A-166 | 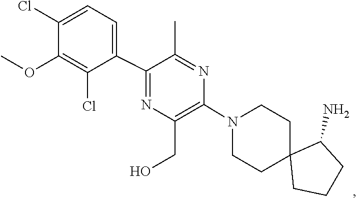 [SMILES: COc1c(Cl)ccc(-c2nc(CO)c(N3CCC4(CCC[C@H]4N)CC3)nc2C)c1Cl] |
| A-167 | 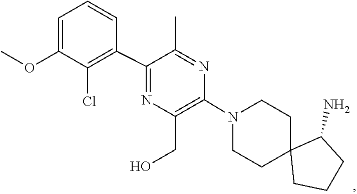 [SMILES: COc1cccc(-c2nc(CO)c(N3CCC4(CCC[C@H]4N)CC3)nc2C)c1Cl] |
| A-168 | 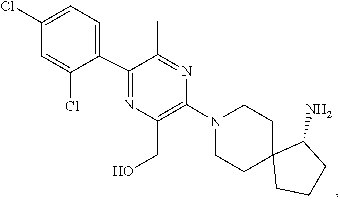 [SMILES: Cc1nc(N2CCC3(CCC[C@H]3N)CC2)c(CO)nc1-c1ccc(Cl)cc1Cl] |
| A-169 | 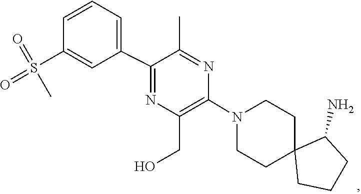 [SMILES: Cc1nc(N2CCC3(CCC[C@H]3N)CC2)c(CO)nc1-c1cccc(S(C)(=O)=O)c1] |
| A-170 | 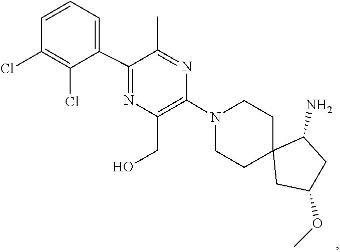 [SMILES: CO[C@H]1C[C@@H](N)C2(CCN(c3nc(C)c(-c4cccc(Cl)c4Cl)nc3CO)CC2)C1] |
| A-171 | 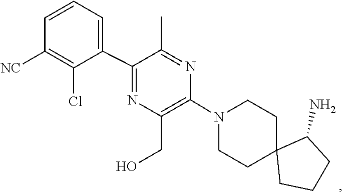 [SMILES: Cc1nc(N2CCC3(CCC[C@H]3N)CC2)c(CO)nc1-c1cccc(C#N)c1Cl] |
| A-172 | 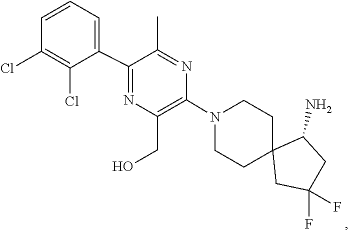 [SMILES: Cc1nc(N2CCC3(CC2)CC(F)(F)C[C@H]3N)c(CO)nc1-c1cccc(Cl)c1Cl] |
| A-173 | 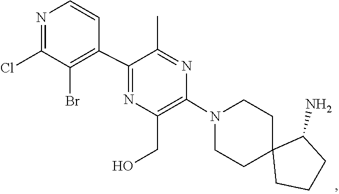 [SMILES: Cc1nc(N2CCC3(CCC[C@H]3N)CC2)c(CO)nc1-c1ccnc(Cl)c1Br] |
| A-174 | 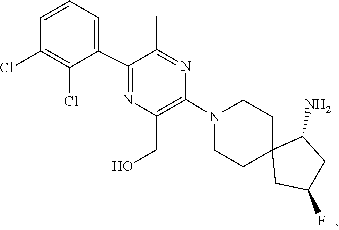 [SMILES: Cc1nc(N2CCC3(CC2)C[C@H](F)C[C@H]3N)c(CO)nc1-c1cccc(Cl)c1Cl] |
| A-175 | 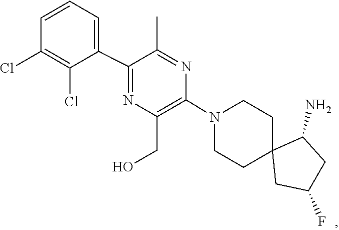 [SMILES: Cc1nc(N2CCC3(CC2)C[C@@H](F)C[C@H]3N)c(CO)nc1-c1cccc(Cl)c1Cl] |
| A-176 | 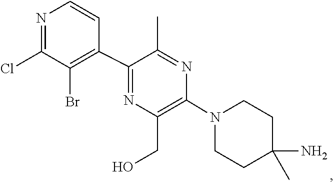 [SMILES: Cc1nc(N2CCC(C)(N)CC2)c(CO)nc1-c1ccnc(Cl)c1Br] |
| A-177 | 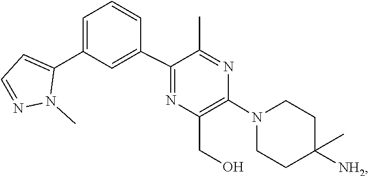 [SMILES: Cc1nc(N2CCC(C)(N)CC2)c(CO)nc1-c1cccc(-c2ccnn2C)c1] |
| A-178 | 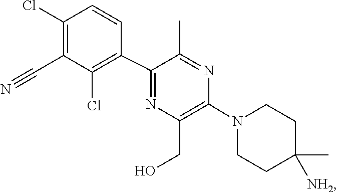 [SMILES: Cc1nc(N2CCC(C)(N)CC2)c(CO)nc1-c1ccc(Cl)c(C#N)c1Cl] |
| A-179 | 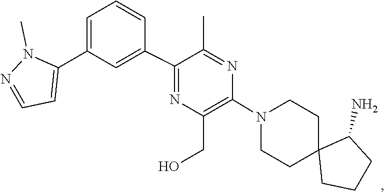 [SMILES: Cc1nc(N2CCC3(CCC[C@H]3N)CC2)c(CO)nc1-c1cccc(-c2ccnn2C)c1] |
| A-180 | 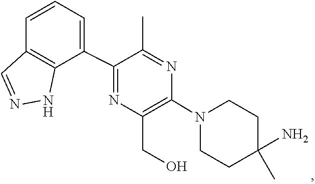 [SMILES: Cc1nc(N2CCC(C)(N)CC2)c(CO)nc1-c1cccc2cn[nH]c12] |
| A-181 | 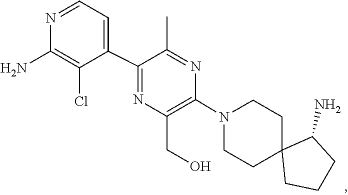 [SMILES: Cc1nc(N2CCC3(CCC[C@H]3N)CC2)c(CO)nc1-c1ccnc(N)c1Cl] |
| A-182 | 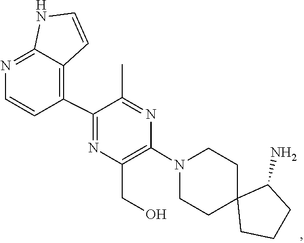 [SMILES: Cc1nc(N2CCC3(CCC[C@H]3N)CC2)c(CO)nc1-c1ccnc2[nH]ccc12] |
| A-183 | 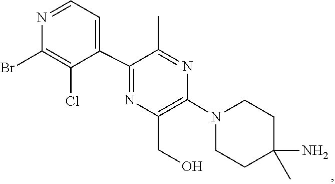 [SMILES: Cc1nc(N2CCC(C)(N)CC2)c(CO)nc1-c1ccnc(Br)c1Cl] |
| A-184 | 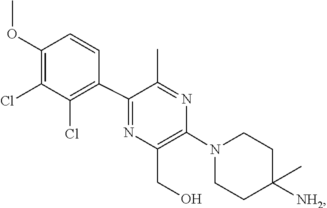 [SMILES: COc1ccc(-c2nc(CO)c(N3CCC(C)(N)CC3)nc2C)c(Cl)c1Cl] |
| A-185 | 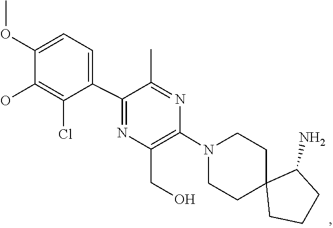 [SMILES: COc1ccc(-c2nc(CO)c(N3CCC4(CCC[C@H]4N)CC3)nc2C)c(Cl)c1O] ' |
| A-186 | 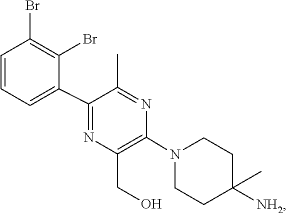 [SMILES: Cc1nc(N2CCC(C)(N)CC2)c(CO)nc1-c1cccc(Br)c1Br] |
| A-187 | 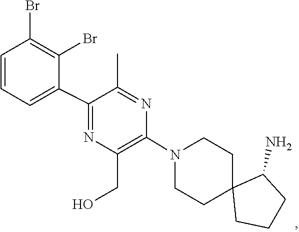 [SMILES: Cc1nc(N2CCC3(CCC[C@H]3N)CC2)c(CO)nc1-c1cccc(Br)c1Br] |
| A-188 | 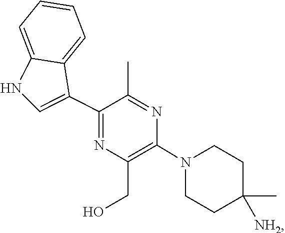 [SMILES: Cc1nc(N2CCC(C)(N)CC2)c(CO)nc1-c1c[nH]c2ccccc12] |
| A-189 | 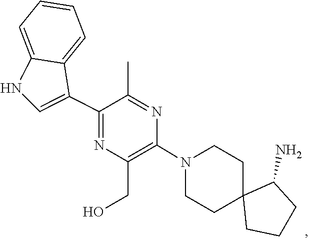 [SMILES: Cc1nc(N2CCC3(CCC[C@H]3N)CC2)c(CO)nc1-c1c[nH]c2ccccc12] |
| A-190 | 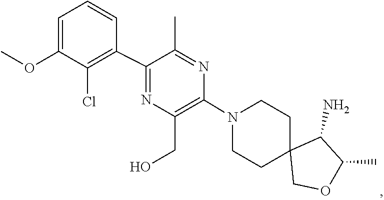 |
| A-191 | 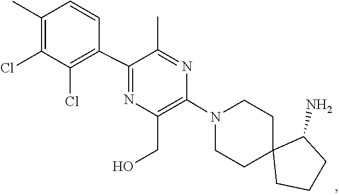 [SMILES: Cc1ccc(-c2nc(CO)c(N3CCC4(CCC[C@H]4N)CC3)nc2C)c(Cl)c1Cl] |
| A-192 | 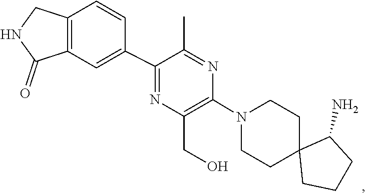 [SMILES: Cc1nc(N2CCC3(CCC[C@H]3N)CC2)c(CO)nc1-c1ccc2c(c1)C(=O)NC2] |
| A-193 | 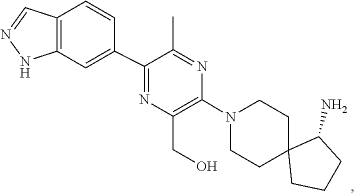 [SMILES: Cc1nc(N2CCC3(CCC[C@H]3N)CC2)c(CO)nc1-c1ccc2cn[nH]c2c1] |
| A-194 | 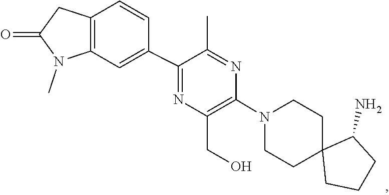 [SMILES: Cc1nc(N2CCC3(CCC[C@H]3N)CC2)c(CO)nc1-c1ccc2c(c1)N(C)C(=O)C2] |
| A-195 | 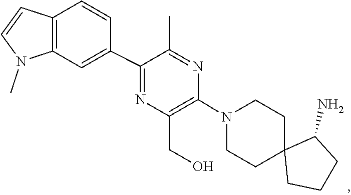 [SMILES: Cc1nc(N2CCC3(CCC[C@H]3N)CC2)c(CO)nc1-c1ccc2ccn(C)c2c1] |
| A-196 | 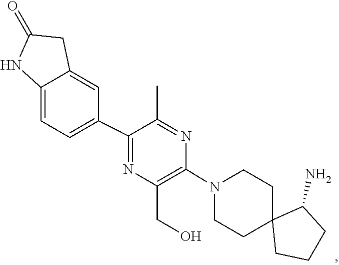 [SMILES: Cc1nc(N2CCC3(CCC[C@H]3N)CC2)c(CO)nc1-c1ccc2c(c1)CC(=O)N2] |
| A-197 | 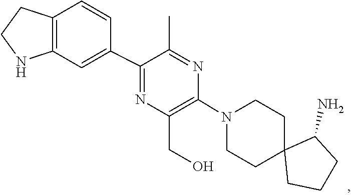 [SMILES: Cc1nc(N2CCC3(CCC[C@H]3N)CC2)c(CO)nc1-c1ccc2c(c1)NCC2] |
| A-198 | 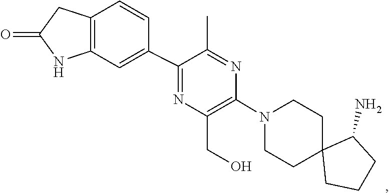 [SMILES: Cc1nc(N2CCC3(CCC[C@H]3N)CC2)c(CO)nc1-c1ccc2c(c1)NC(=O)C2] |
| A-199 | 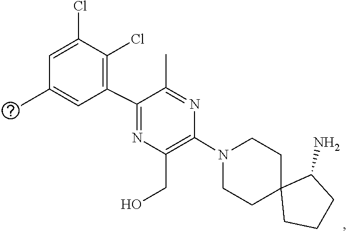 [SMILES: Cc1cc(Cl)c(Cl)c(-c2nc(CO)c(N3CCC4(CCC[C@H]4N)CC3)nc2C)c1] |
| A-200 | 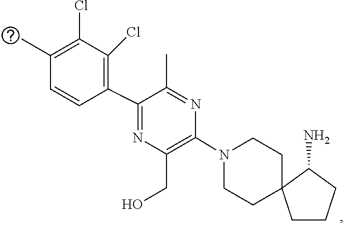 [SMILES: Cc1ccc(-c2nc(CO)c(N3CCC4(CCC[C@H]4N)CC3)nc2C)c(Cl)c1Cl] |
| A-201 | 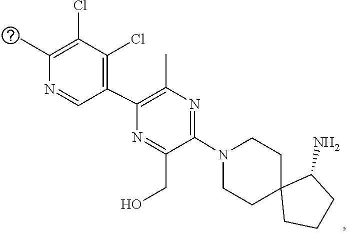 [SMILES: Cc1nc(N2CCC3(CCC[C@H]3N)CC2)c(CO)nc1-c1cnc(C)c(Cl)c1Cl] |
| A-202 | 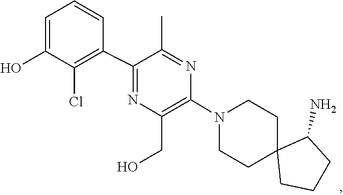 [SMILES: Cc1nc(N2CCC3(CCC[C@H]3N)CC2)c(CO)nc1-c1cccc(O)c1Cl] |
| A-203 | 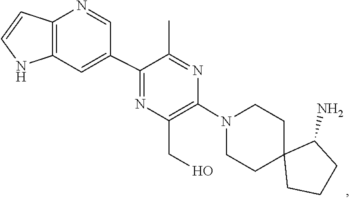 [SMILES: Cc1nc(N2CCC3(CCC[C@H]3N)CC2)c(CO)nc1-c1cnc2cc[nH]c2c1] |
| A-204 | 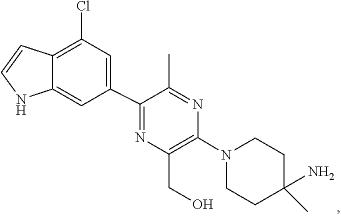 [SMILES: Cc1nc(N2CCC(C)(N)CC2)c(CO)nc1-c1cc(Cl)c2cc[nH]c2c1] |
| A-205 | 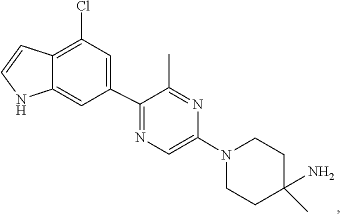 [SMILES: Cc1nc(N2CCC(C)(N)CC2)cnc1-c1cc(Cl)c2cc[nH]c2c1] |
| A-206 | 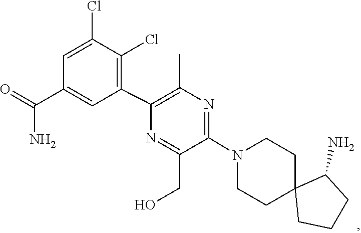 [SMILES: Cc1nc(N2CCC3(CCC[C@H]3N)CC2)c(CO)nc1-c1cc(C(N)=O)cc(Cl)c1Cl] |
| A-207 | 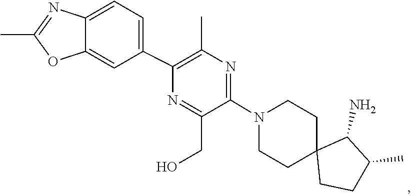 |
| A-208 | 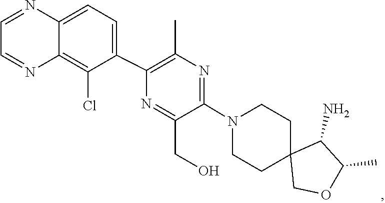 [SMILES: Cc1nc(N2CCC3(CC2)CO[C@@H](C)[C@H]3N)c(CO)nc1-c1ccc2nccnc2c1Cl] |
| A-209 | 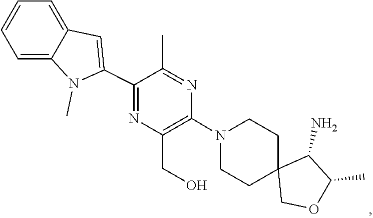 [SMILES: Cc1nc(N2CCC3(CC2)CO[C@@H](C)[C@H]3N)c(CO)nc1-c1cc2ccccc2n1C] |
| A-210 | 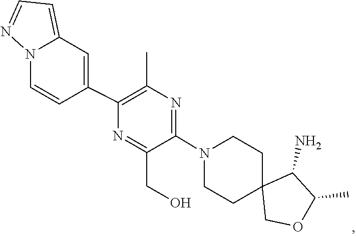 [SMILES: Cc1nc(N2CCC3(CC2)CO[C@@H](C)[C@H]3N)c(CO)nc1-c1ccn2nccc2c1] |
| A-211 | 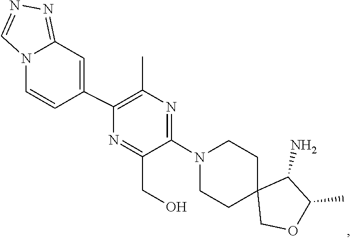 [SMILES: Cc1nc(N2CCC3(CC2)CO[C@@H](C)[C@H]3N)c(CO)nc1-c1ccn2cnnc2c1] |
| A-212 | 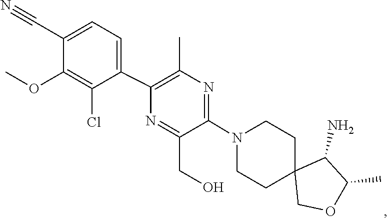 |
| A-213 | 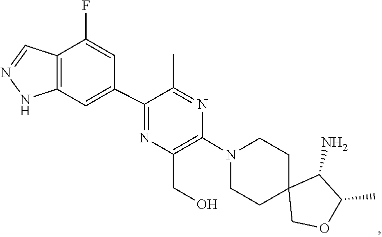 [SMILES: Cc1nc(N2CCC3(CC2)CO[C@@H](C)[C@H]3N)c(CO)nc1-c1cc(F)c2cn[nH]c2c1] |
| A-214 | 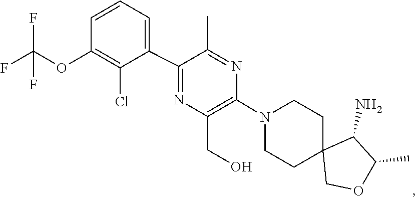 [SMILES: Cc1nc(N2CCC3(CC2)CO[C@@H](C)[C@H]3N)c(CO)nc1-c1cccc(OC(F)(F)F)c1Cl] |
| A-215 | 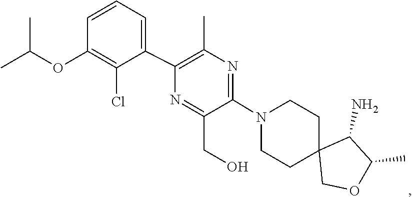 [SMILES: Cc1nc(N2CCC3(CC2)CO[C@@H](C)[C@H]3N)c(CO)nc1-c1cccc(OC(C)C)c1Cl] |
| A-216 | 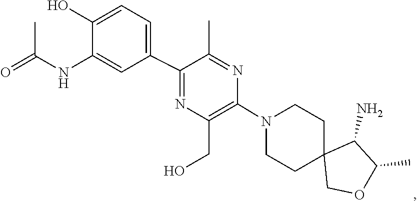 [SMILES: CC(=O)Nc1cc(-c2nc(CO)c(N3CCC4(CC3)CO[C@@H](C)[C@H]4N)nc2C)ccc1O] |
| A-217 | 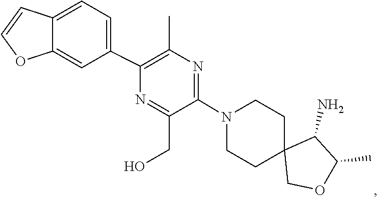 [SMILES: Cc1nc(N2CCC3(CC2)CO[C@@H](C)[C@H]3N)c(CO)nc1-c1ccc2ccoc2c1] |
| A-218 | 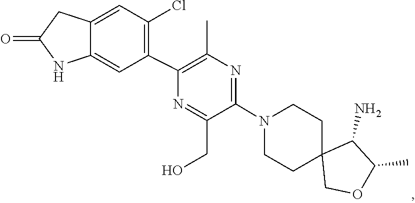 [SMILES: Cc1nc(N2CCC3(CC2)CO[C@@H](C)[C@H]3N)c(CO)nc1-c1cc2c(cc1Cl)CC(=O)N2] |
| A-219 | 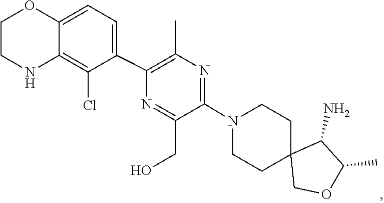 [SMILES: Cc1nc(N2CCC3(CC2)CO[C@@H](C)[C@H]3N)c(CO)nc1-c1ccc2c(c1Cl)NCCO2] |
| A-220 | 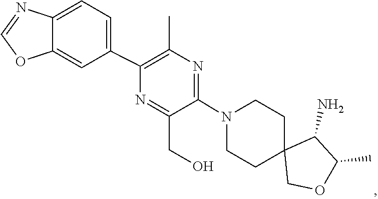 [SMILES: Cc1nc(N2CCC3(CC2)CO[C@@H](C)[C@H]3N)c(CO)nc1-c1ccc2ncoc2c1] |
| A-221 | 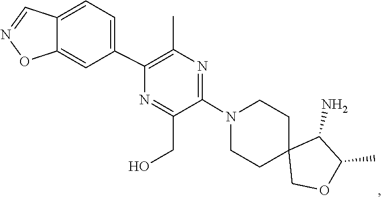 [SMILES: Cc1nc(N2CCC3(CC2)CO[C@@H](C)[C@H]3N)c(CO)nc1-c1ccc2cnoc2c1] |
| A-222 | 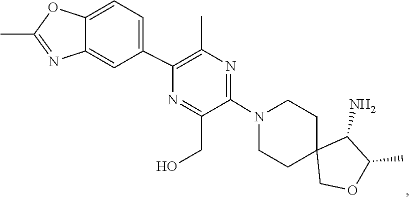 |
| A-223 | 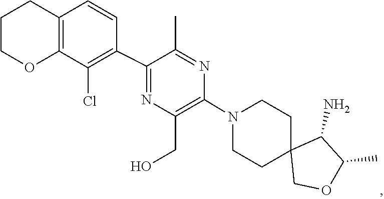 [SMILES: Cc1nc(N2CCC3(CC2)CO[C@@H](C)[C@H]3N)c(CO)nc1-c1ccc2c(c1Cl)OCCC2] |
| A-224 | 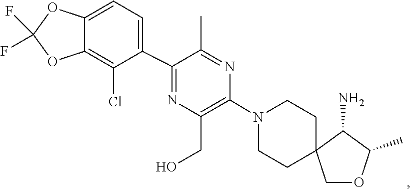 [SMILES: Cc1nc(N2CCC3(CC2)CO[C@@H](C)[C@H]3N)c(CO)nc1-c1ccc2c(c1Cl)OC(F)(F)O2] |
| A-225 | 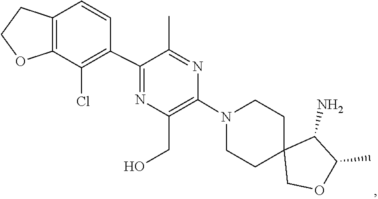 [SMILES: Cc1nc(N2CCC3(CC2)CO[C@@H](C)[C@H]3N)c(CO)nc1-c1ccc2c(c1Cl)OCC2] |
| A-226 | 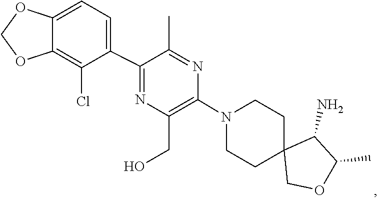 [SMILES: Cc1nc(N2CCC3(CC2)CO[C@@H](C)[C@H]3N)c(CO)nc1-c1ccc2c(c1Cl)OCO2] |
| A-227 | 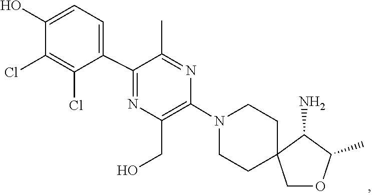 [SMILES: Cc1nc(N2CCC3(CC2)CO[C@@H](C)[C@H]3N)c(CO)nc1-c1ccc(O)c(Cl)c1Cl] |
| A-228 | 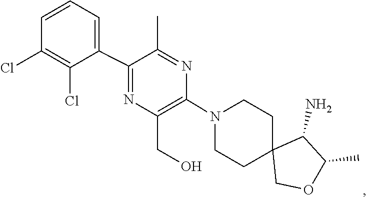 [SMILES: Cc1nc(N2CCC3(CC2)CO[C@@H](C)[C@H]3N)c(CO)nc1-c1cccc(Cl)c1Cl] |
| A-229 | 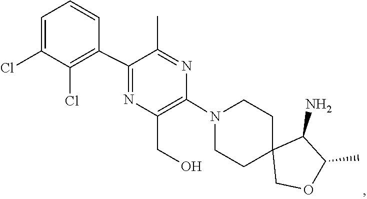 [SMILES: Cc1nc(N2CCC3(CC2)CO[C@@H](C)[C@@H]3N)c(CO)nc1-c1cccc(Cl)c1Cl] |
| A-230 | 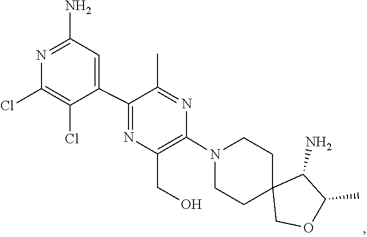 [SMILES: Cc1nc(N2CCC3(CC2)CO[C@@H](C)[C@H]3N)c(CO)nc1-c1cc(N)nc(Cl)c1Cl] |
| A-231 | 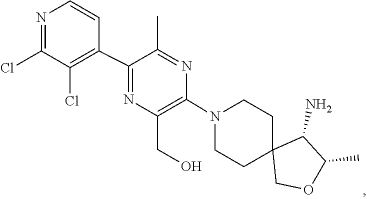 [SMILES: Cc1nc(N2CCC3(CC2)CO[C@@H](C)[C@H]3N)c(CO)nc1-c1ccnc(Cl)c1Cl] |
| A-232 | 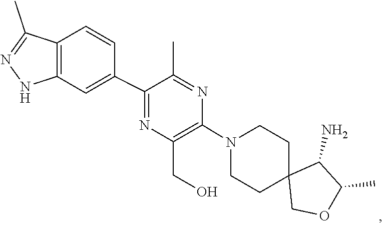 [SMILES: Cc1nc(N2CCC3(CC2)CO[C@@H](C)[C@H]3N)c(CO)nc1-c1ccc2c(C)n[nH]c2c1] |
| A-233 | 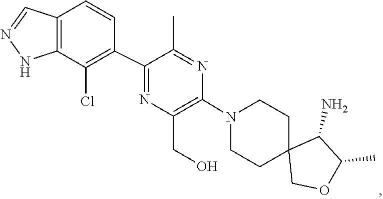 [SMILES: Cc1nc(N2CCC3(CC2)CO[C@@H](C)[C@H]3N)c(CO)nc1-c1ccc2cn[nH]c2c1Cl] |
| A-234 | 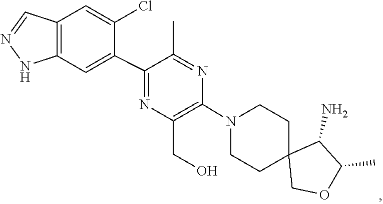 [SMILES: Cc1nc(N2CCC3(CC2)CO[C@@H](C)[C@H]3N)c(CO)nc1-c1cc2[nH]ncc2cc1Cl] |
| A-235 | 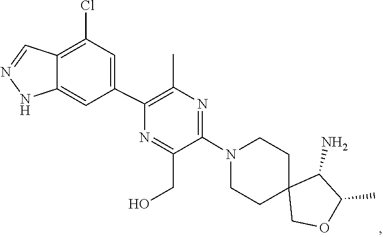 [SMILES: Cc1nc(N2CCC3(CC2)CO[C@@H](C)[C@H]3N)c(CO)nc1-c1cc(Cl)c2cn[nH]c2c1] |
| A-236 | 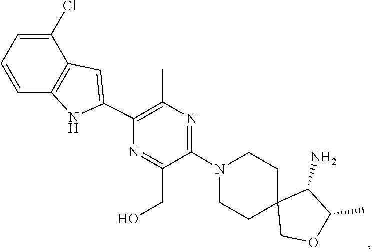 [SMILES: Cc1nc(N2CCC3(CC2)CO[C@@H](C)[C@H]3N)c(CO)nc1-c1cc2c(Cl)cccc2[nH]1] |
| A-237 | 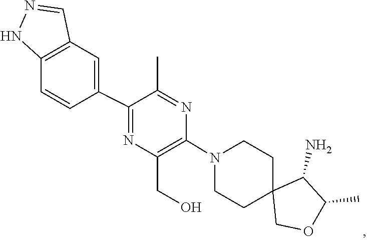 [SMILES: Cc1nc(N2CCC3(CC2)CO[C@@H](C)[C@H]3N)c(CO)nc1-c1ccc2[nH]ncc2c1] |
| A-238 | 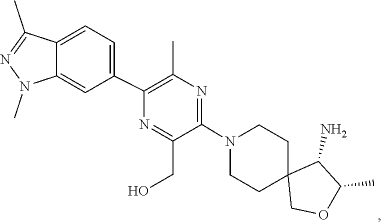 [SMILES: Cc1nc(N2CCC3(CC2)CO[C@@H](C)[C@H]3N)c(CO)nc1-c1ccc2c(C)nn(C)c2c1] |
| A-239 | 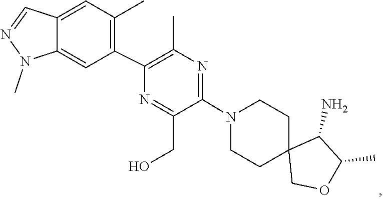 [SMILES: Cc1cc2cnn(C)c2cc1-c1nc(CO)c(N2CCC3(CC2)CO[C@@H](C)[C@H]3N)nc1C] |
| A-240 | 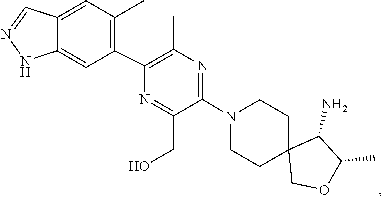 [SMILES: Cc1cc2cn[nH]c2cc1-c1nc(CO)c(N2CCC3(CC2)CO[C@@H](C)[C@H]3N)nc1C] |
| A-241 | 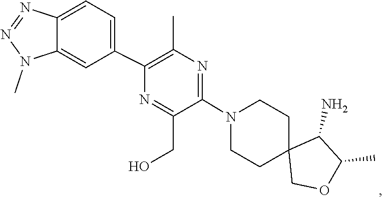 [SMILES: Cc1nc(N2CCC3(CC2)CO[C@@H](C)[C@H]3N)c(CO)nc1-c1ccc2nnn(C)c2c1] |
| A-242 | 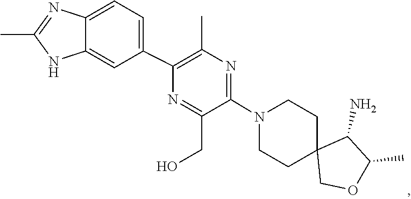 |
| A-243 | 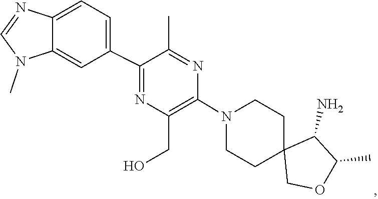 [SMILES: Cc1nc(N2CCC3(CC2)CO[C@@H](C)[C@H]3N)c(CO)nc1-c1ccc2ncn(C)c2c1] |
| A-244 | 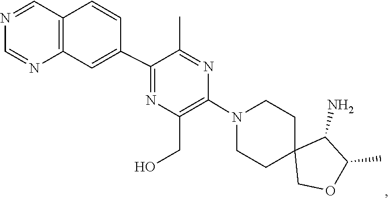 [SMILES: Cc1nc(N2CCC3(CC2)CO[C@@H](C)[C@H]3N)c(CO)nc1-c1ccc2cncnc2c1] |
| A-245 | 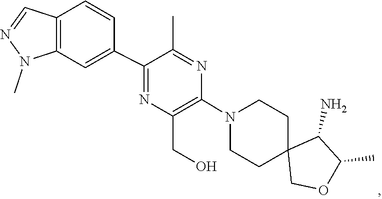 [SMILES: Cc1nc(N2CCC3(CC2)CO[C@@H](C)[C@H]3N)c(CO)nc1-c1ccc2cnn(C)c2c1] . |
| A-246 | 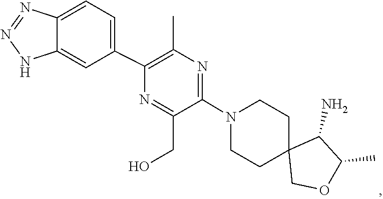 [SMILES: Cc1nc(N2CCC3(CC2)CO[C@@H](C)[C@H]3N)c(CO)nc1-c1ccc2nn[nH]c2c1] |
| A-247 | 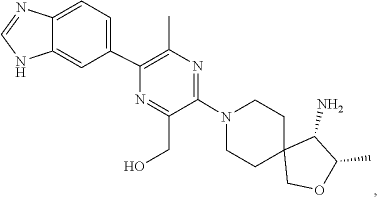 [SMILES: Cc1nc(N2CCC3(CC2)CO[C@@H](C)[C@H]3N)c(CO)nc1-c1ccc2nc[nH]c2c1] |
| A-248 | 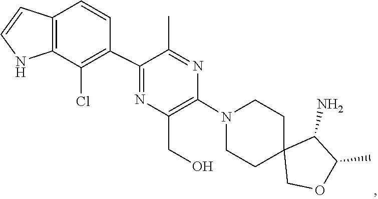 [SMILES: Cc1nc(N2CCC3(CC2)CO[C@@H](C)[C@H]3N)c(CO)nc1-c1ccc2cc[nH]c2c1Cl] |
| A-249 | 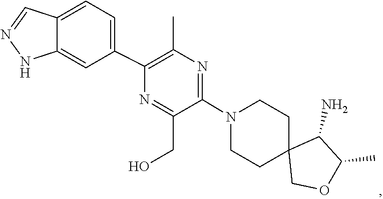 [SMILES: Cc1nc(N2CCC3(CC2)CO[C@@H](C)[C@H]3N)c(CO)nc1-c1ccc2cn[nH]c2c1] |
| A-250 | 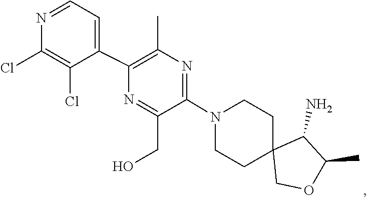 [SMILES: Cc1nc(N2CCC3(CC2)CO[C@H](C)[C@H]3N)c(CO)nc1-c1ccnc(Cl)c1Cl] |
| A-251 | 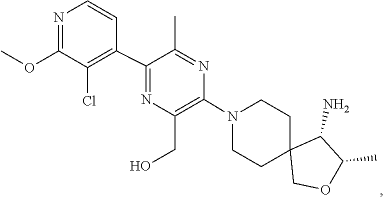 |
| A-252 | 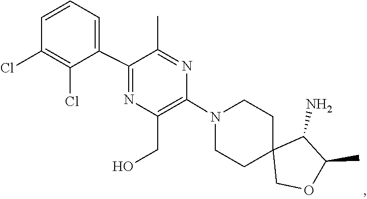 [SMILES: Cc1nc(N2CCC3(CC2)CO[C@H](C)[C@H]3N)c(CO)nc1-c1cccc(Cl)c1Cl] |
| A-253 | 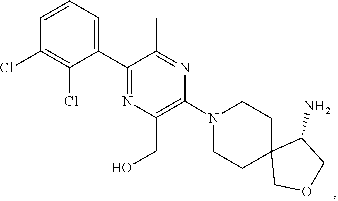 [SMILES: Cc1nc(N2CCC3(CC2)COC[C@H]3N)c(CO)nc1-c1cccc(Cl)c1Cl] |
| A-254 | 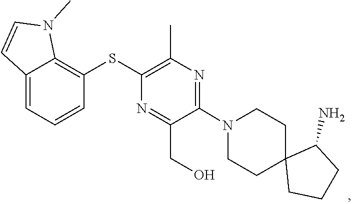 [SMILES: Cc1nc(N2CCC3(CCC[C@H]3N)CC2)c(CO)nc1Sc1cccc2ccn(C)c12] |
| A-255 | 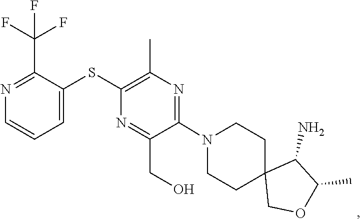 [SMILES: Cc1nc(N2CCC3(CC2)CO[C@@H](C)[C@H]3N)c(CO)nc1Sc1cccnc1C(F)(F)F] |
| A-256 | 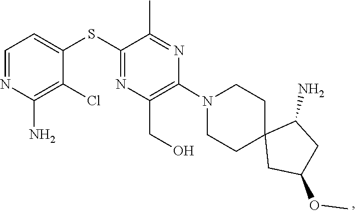 [SMILES: CO[C@@H]1C[C@@H](N)C2(CCN(c3nc(C)c(Sc4ccnc(N)c4Cl)nc3CO)CC2)C1] |
| A-257 | 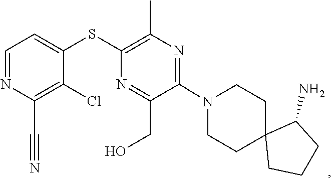 [SMILES: Cc1nc(N2CCC3(CCC[C@H]3N)CC2)c(CO)nc1Sc1ccnc(C#N)c1Cl] |
| A-258 | 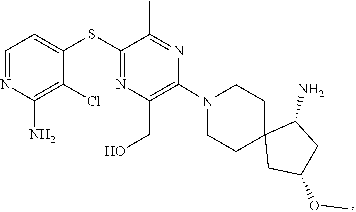 [SMILES: CO[C@H]1C[C@@H](N)C2(CCN(c3nc(C)c(Sc4ccnc(N)c4Cl)nc3CO)CC2)C1] |
| A-259 | 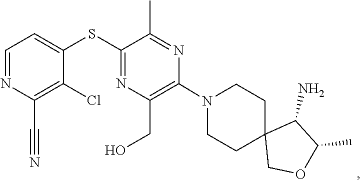 |
| A-260 | 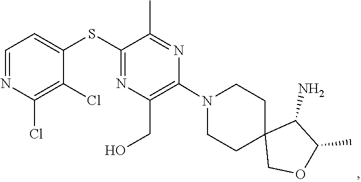 [SMILES: Cc1nc(N2CCC3(CC2)CO[C@@H](C)[C@H]3N)c(CO)nc1Sc1ccnc(Cl)c1Cl] |
| A-261 | 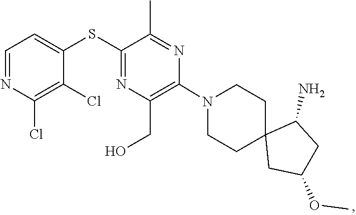 [SMILES: CO[C@H]1C[C@@H](N)C2(CCN(c3nc(C)c(Sc4ccnc(Cl)c4Cl)nc3CO)CC2)C1] |
| A-262 | 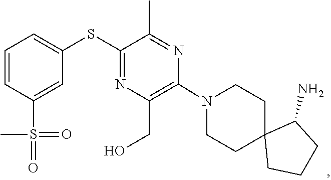 [SMILES: Cc1nc(N2CCC3(CCC[C@H]3N)CC2)c(CO)nc1Sc1cccc(S(C)(=O)=O)c1] |
| A-263 | 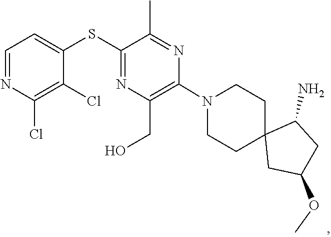 [SMILES: CO[C@@H]1C[C@@H](N)C2(CCN(c3nc(C)c(Sc4ccnc(Cl)c4Cl)nc3CO)CC2)C1] |
| A-264 | 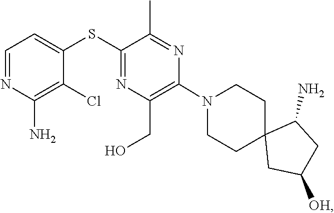 [SMILES: Cc1nc(N2CCC3(CC2)C[C@H](O)C[C@H]3N)c(CO)nc1Sc1ccnc(N)c1Cl] |
| A-265 | 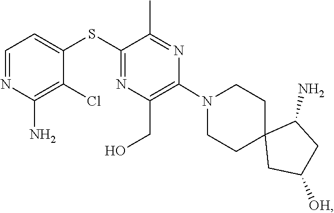 [SMILES: Cc1nc(N2CCC3(CC2)C[C@@H](O)C[C@H]3N)c(CO)nc1Sc1ccnc(N)c1Cl] |
| A-266 | 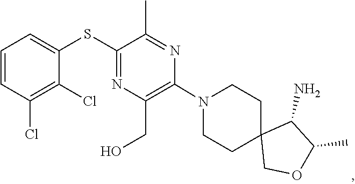 [SMILES: Cc1nc(N2CCC3(CC2)CO[C@@H](C)[C@H]3N)c(CO)nc1Sc1cccc(Cl)c1Cl] |
| A-267 | 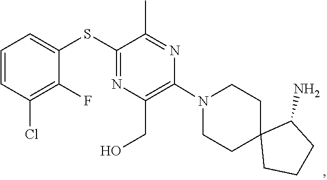 [SMILES: Cc1nc(N2CCC3(CCC[C@H]3N)CC2)c(CO)nc1Sc1cccc(Cl)c1F] |
| A-268 | 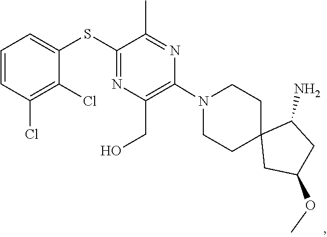 [SMILES: CO[C@@H]1C[C@@H](N)C2(CCN(c3nc(C)c(Sc4cccc(Cl)c4Cl)nc3CO)CC2)C1] |
| A-269 | 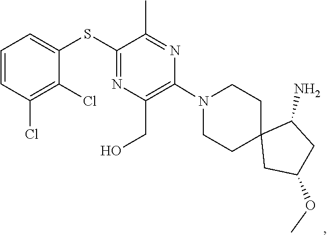 [SMILES: CO[C@H]1C[C@@H](N)C2(CCN(c3nc(C)c(Sc4cccc(Cl)c4Cl)nc3CO)CC2)C1] |
| A-270 | 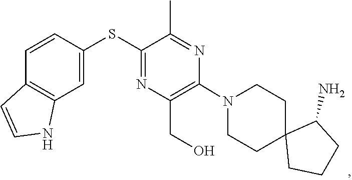 [SMILES: Cc1nc(N2CCC3(CCC[C@H]3N)CC2)c(CO)nc1Sc1ccc2cc[nH]c2c1] |
| A-271 | 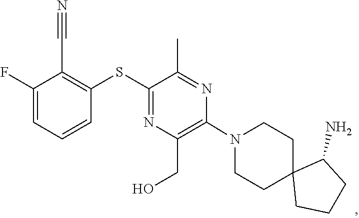 [SMILES: Cc1nc(N2CCC3(CCC[C@H]3N)CC2)c(CO)nc1Sc1cccc(F)c1C#N] |
| A-272 | 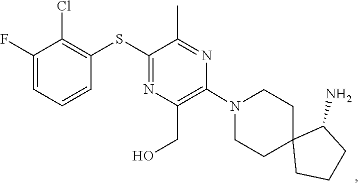 [SMILES: Cc1nc(N2CCC3(CCC[C@H]3N)CC2)c(CO)nc1Sc1cccc(F)c1Cl] |
| A-273 | 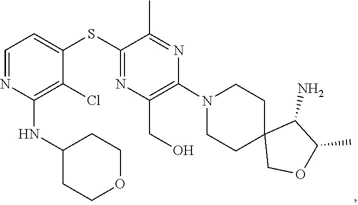 [SMILES: Cc1nc(N2CCC3(CC2)CO[C@@H](C)[C@H]3N)c(CO)nc1Sc1ccnc(NC2CCOCC2)c1Cl] |
| A-274 | 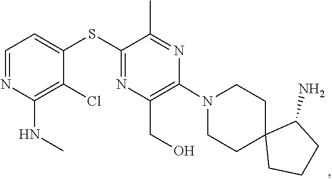 [SMILES: CNc1nccc(Sc2nc(CO)c(N3CCC4(CCC[C@H]4N)CC3)nc2C)c1Cl] |
| A-275 | 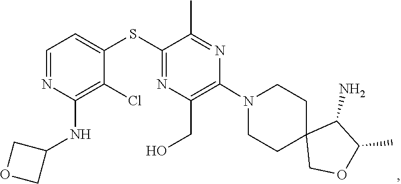 [SMILES: Cc1nc(N2CCC3(CC2)CO[C@@H](C)[C@H]3N)c(CO)nc1Sc1ccnc(NC2COC2)c1Cl] |
| A-276 | 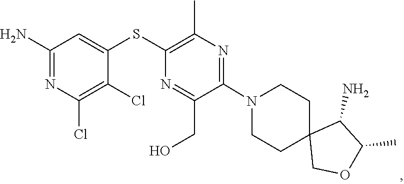 [SMILES: Cc1nc(N2CCC3(CC2)CO[C@@H](C)[C@H]3N)c(CO)nc1Sc1cc(N)nc(Cl)c1Cl] |
| A-277 | 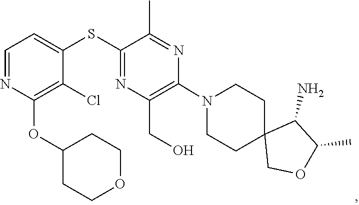 [SMILES: Cc1nc(N2CCC3(CC2)CO[C@@H](C)[C@H]3N)c(CO)nc1Sc1ccnc(OC2CCOCC2)c1Cl] |
| A-278 | 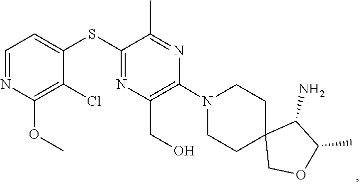 [SMILES: COc1nccc(Sc2nc(CO)c(N3CCC4(CC3)CO[C@@H](C)[C@H]4N)nc2C)c1Cl] |
| A-279 | 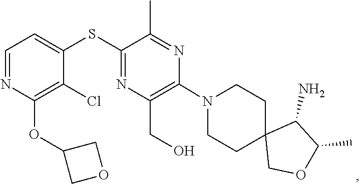 [SMILES: Cc1nc(N2CCC3(CC2)CO[C@@H](C)[C@H]3N)c(CO)nc1Sc1ccnc(OC2COC2)c1Cl] |
| A-280 | 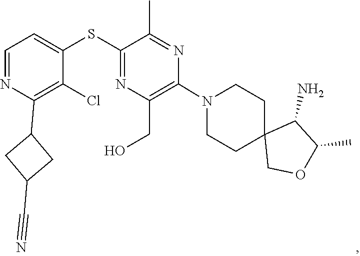 [SMILES: Cc1nc(N2CCC3(CC2)CO[C@@H](C)[C@H]3N)c(CO)nc1Sc1ccnc(C2CC(C#N)C2)c1Cl] |
| A-281 | 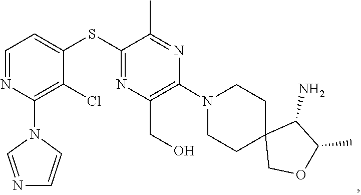 [SMILES: Cc1nc(N2CCC3(CC2)CO[C@@H](C)[C@H]3N)c(CO)nc1Sc1ccnc(-n2ccnc2)c1Cl] |
| A-282 | 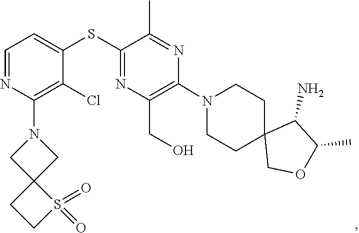 [SMILES: Cc1nc(N2CCC3(CC2)CO[C@@H](C)[C@H]3N)c(CO)nc1Sc1ccnc(N2CC3(CCS3(=O)=O)C2)c1Cl] |
| A-283 | 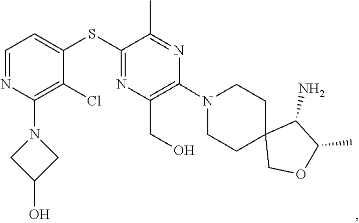 [SMILES: Cc1nc(N2CCC3(CC2)CO[C@@H](C)[C@H]3N)c(CO)nc1Sc1ccnc(N2CC(O)C2)c1Cl] |
| A-284 | 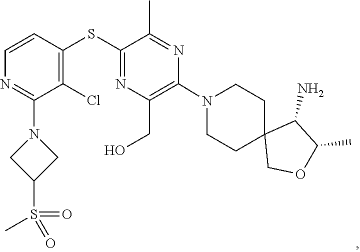 [SMILES: Cc1nc(N2CCC3(CC2)CO[C@@H](C)[C@H]3N)c(CO)nc1Sc1ccnc(N2CC(S(C)(=O)=O)C2)c1Cl] |
| A-285 | 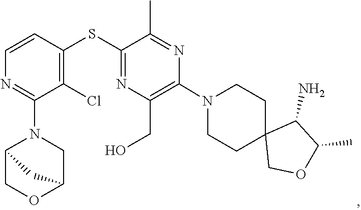 [SMILES: Cc1nc(N2CCC3(CC2)CO[C@@H](C)[C@H]3N)c(CO)nc1Sc1ccnc(N2C[C@@H]3C[C@H]2CO3)c1Cl] |
| A-286 | 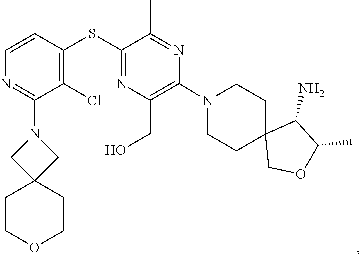 [SMILES: Cc1nc(N2CCC3(CC2)CO[C@@H](C)[C@H]3N)c(CO)nc1Sc1ccnc(N2CC3(CCOCC3)C2)c1Cl] |
| A-287 | 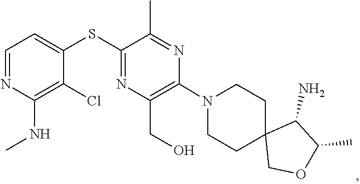 [SMILES: CNc1nccc(Sc2nc(CO)c(N3CCC4(CC3)CO[C@@H](C)[C@H]4N)nc2C)c1Cl] |
| A-288 | 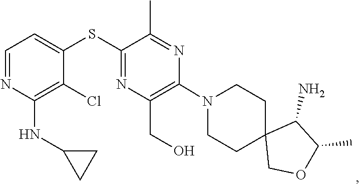 [SMILES: Cc1nc(N2CCC3(CC2)CO[C@@H](C)[C@H]3N)c(CO)nc1Sc1ccnc(NC2CC2)c1Cl] |
| A-289 | 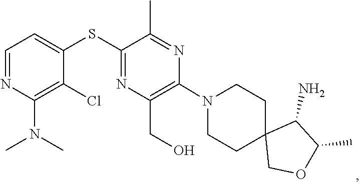 [SMILES: Cc1nc(N2CCC3(CC2)CO[C@@H](C)[C@H]3N)c(CO)nc1Sc1ccnc(N(C)C)c1Cl] |
| A-290 | 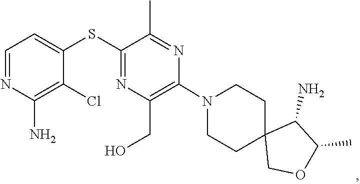 [SMILES: Cc1nc(N2CCC3(CC2)CO[C@@H](C)[C@H]3N)c(CO)nc1Sc1ccnc(N)c1Cl] |
| A-291 | 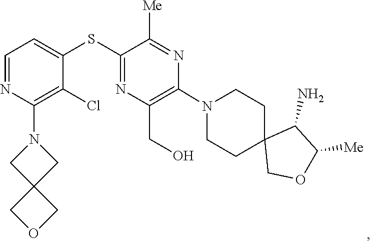 [SMILES: Cc1nc(N2CCC3(CC2)CO[C@@H](C)[C@H]3N)c(CO)nc1Sc1ccnc(N2CC3(COC3)C2)c1Cl] |
| A-292 | 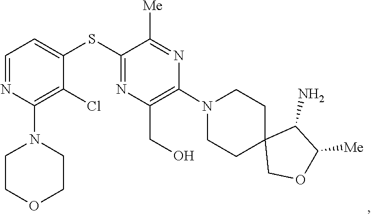 [SMILES: Cc1nc(N2CCC3(CC2)CO[C@@H](C)[C@H]3N)c(CO)nc1Sc1ccnc(N2CCOCC2)c1Cl] |
| A-293 | 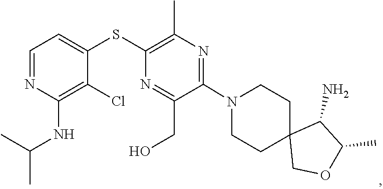 [SMILES: Cc1nc(N2CCC3(CC2)CO[C@@H](C)[C@H]3N)c(CO)nc1Sc1ccnc(NC(C)C)c1Cl] |
| A-294 | 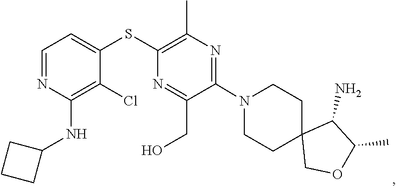 [SMILES: Cc1nc(N2CCC3(CC2)CO[C@@H](C)[C@H]3N)c(CO)nc1Sc1ccnc(NC2CCC2)c1Cl] |
| A-295 | 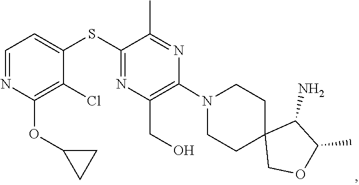 [SMILES: Cc1nc(N2CCC3(CC2)CO[C@@H](C)[C@H]3N)c(CO)nc1Sc1ccnc(OC2CC2)c1Cl] |
| A-296 | 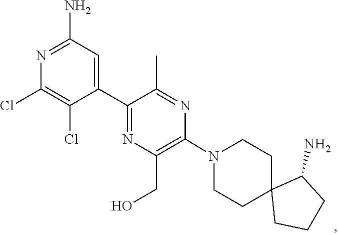 [SMILES: Cc1nc(N2CCC3(CCC[C@H]3N)CC2)c(CO)nc1-c1cc(N)nc(Cl)c1Cl] |
| A-297 | 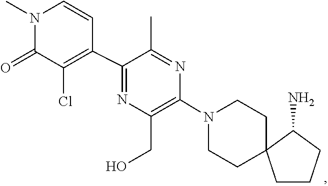 [SMILES: Cc1nc(N2CCC3(CCC[C@H]3N)CC2)c(CO)nc1-c1ccn(C)c(=O)c1Cl] |
| A-298 | 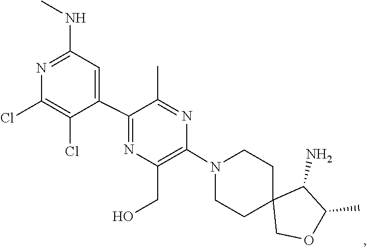 [SMILES: CNc1cc(-c2nc(CO)c(N3CCC4(CC3)CO[C@@H](C)[C@H]4N)nc2C)c(Cl)c(Cl)n1] |
| A-299 | 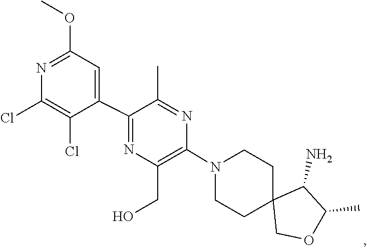 [SMILES: COc1cc(-c2nc(CO)c(N3CCC4(CC3)CO[C@@H](C)[C@H]4N)nc2C)c(Cl)c(Cl)n1] |
| A-300 | 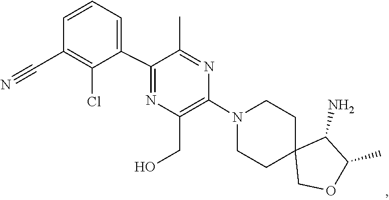 [SMILES: Cc1nc(N2CCC3(CC2)CO[C@@H](C)[C@H]3N)c(CO)nc1-c1cccc(C#N)c1Cl] |
| A-301 | 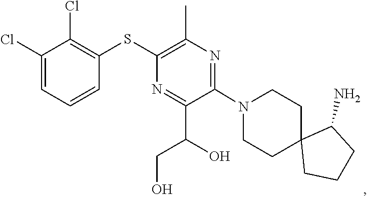 [SMILES: Cc1nc(N2CCC3(CCC[C@H]3N)CC2)c(C(O)CO)nc1Sc1cccc(Cl)c1Cl] |
| A-302 | 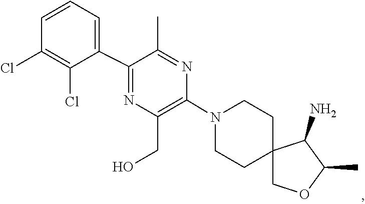 [SMILES: Cc1nc(N2CCC3(CC2)CO[C@H](C)[C@@H]3N)c(CO)nc1-c1cccc(Cl)c1Cl] |
| A-303 | 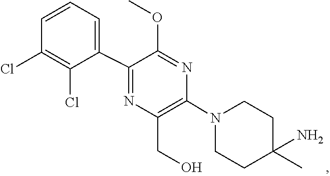 [SMILES: COc1nc(N2CCC(C)(N)CC2)c(CO)nc1-c1cccc(Cl)c1Cl] |
| A-304 | 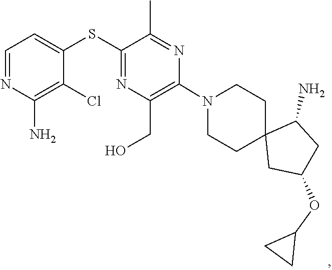 [SMILES: Cc1nc(N2CCC3(CC2)C[C@@H](OC2CC2)C[C@H]3N)c(CO)nc1Sc1ccnc(N)c1Cl] |
| A-305 | 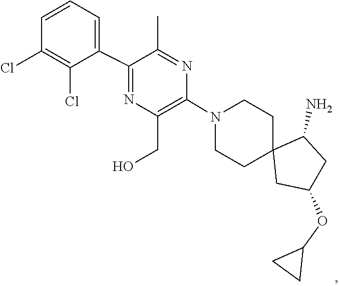 [SMILES: Cc1nc(N2CCC3(CC2)C[C@@H](OC2CC2)C[C@H]3N)c(CO)nc1-c1cccc(Cl)c1Cl] |
| A-306 | 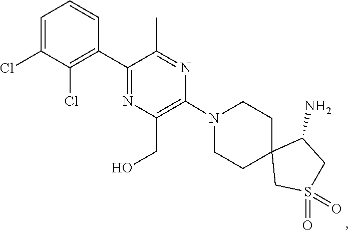 [SMILES: Cc1nc(N2CCC3(CC2)CS(=O)(=O)C[C@H]3N)c(CO)nc1-c1cccc(Cl)c1Cl] |
| A-307 | 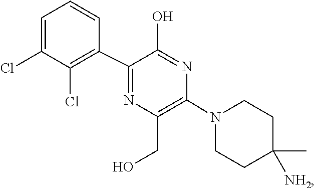 [SMILES: CC1(N)CCN(c2nc(O)c(-c3cccc(Cl)c3Cl)nc2CO)CC1] |
| A-308 | 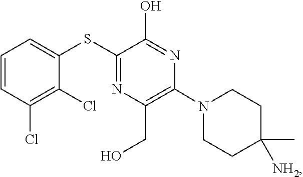 [SMILES: CC1(N)CCN(c2nc(O)c(Sc3cccc(Cl)c3Cl)nc2CO)CC1] |

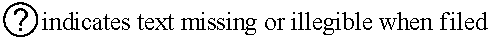

Another aspect of the present disclosure relates to compounds, and pharmaceutically acceptable salts, prodrugs, solvates, hydrates, tautomers, or isomers thereof, in Table 2.

TABLE 2
Structure

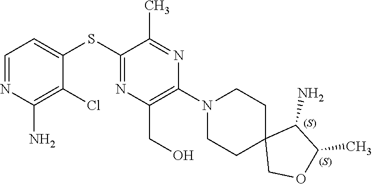

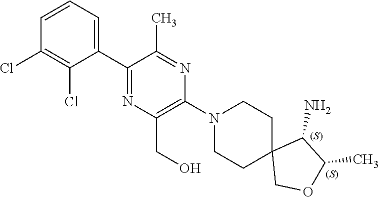

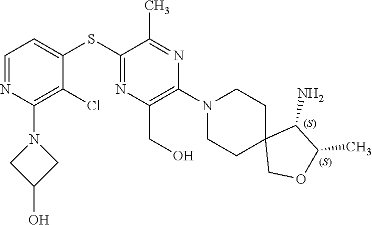

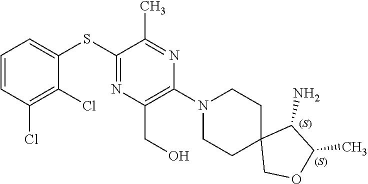

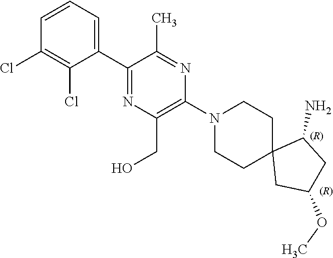

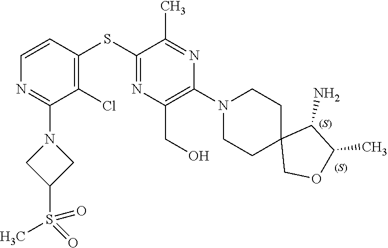

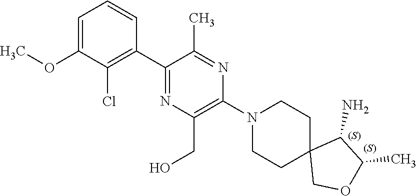

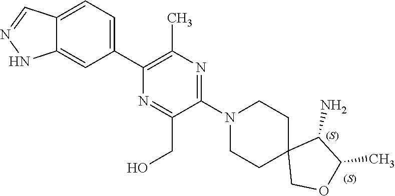

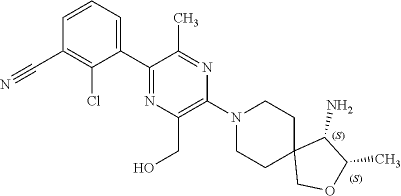

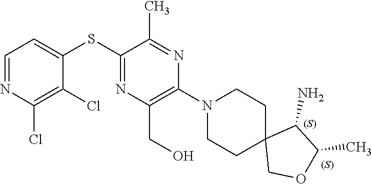

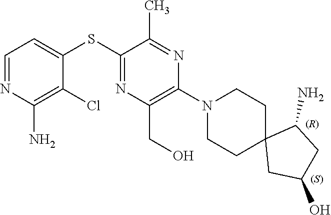

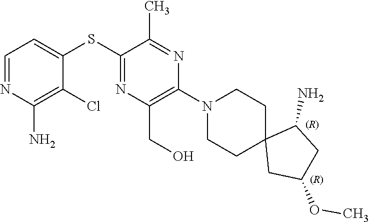

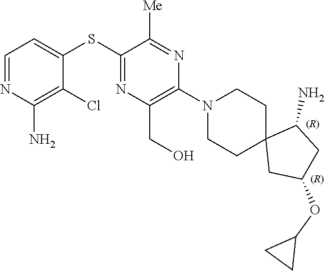

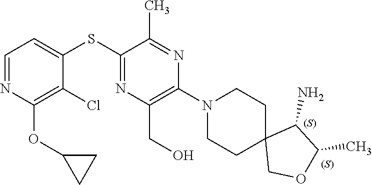

The term “aryl” refers to cyclic, aromatic hydrocarbon groups that have 1 to 2 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. Exemplary substituents include, but are not limited to, —H, halogen, —O—C₁-C₆alkyl, —C₁-C₆alkyl, —OC₂-C₆alkenyl, —OC₂-C₆alkynyl, —C₂-C₆alkenyl, —C₂-C₆alkynyl, —OH, —OP(O)(OH)₂, —OC(O)C₁-C₆alkyl, —C(O)C₁-C₆alkyl, —OC(O)OC₁-C₆alkyl, —NH₂, —NH(C₁-C₆alkyl), —N(C₁-C₆alkyl)₂, —S(O)₂—C₁-C₆alkyl, —S(O)NHC₁-C₆alkyl, and —S(O)N(C₁-C₆alkyl)₂. The substituents can themselves be optionally substituted.

Unless otherwise specifically defined, “heteroaryl” means a monovalent or multivalent monocyclic aromatic radical or a polycyclic aromatic radical of 5 to 24 ring atoms, containing one or more ring heteroatoms selected from N, S, P, and O, the remaining ring atoms being C. Heteroaryl as herein defined also means a bicyclic heteroaromatic group wherein the heteroatom is selected from N, S, P, and O. The aromatic radical is optionally substituted independently with one or more substituents described herein. Examples include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, thiophen-2-yl, quinolyl, benzopyranyl, isothiazolyl, thiazolyl, thiadiazolyl, benzo[d]imidazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, 1-methyl-1H-indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, dihydrobenzoxanyl, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, isoindolin-1-one, indolin-2-one, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydropyrrolo[1,2-a]pyrimidinyl, 3,4-dihydro-2H-λ²-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, 2-methylbenzo[d]oxazolyl, 1,2,3,4-tetrahydropyrrolo[1,2-a]pyrimidyl, 2,3-dihydrobenzofuranyl, benzooxazolyl, benzoisoxazolyl, benzo[d]isoxazolyl, benzo[d]oxazolyl, furo[2,3-b]pyridinyl, benzothiophenyl, 1,5-naphthyridinyl, furo[3,2-b]pyridinyl, [1,2,4]triazolo[1,5-a]pyridinyl, benzo[1,2,3]triazolyl, 1-methyl-1H-benzo[d][1,2,3]triazolyl, imidazo[1,2-a]pyrimidinyl, [1,2,4]triazolo[4,3-b]pyridazinyl, quinoxalinyl, benzo[c][1,2,5]thiadiazolyl, benzo[c][1,2,5]oxadiazolyl, 1,3-dihydro-2H-benzo[d]imidazol-2-one, 3,4-dihydro-2H-pyrazolo[1,5-b][1,2]oxazinyl, 3,4-dihydro-2H-benzo[b][1,4]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, 3H-indolyl, benzo[d][1,3]dioxolyl, pyrazolo[1,5-a]pyridinyl, and derivatives thereof.

“Alkyl” refers to a straight or branched chain saturated hydrocarbon. C₁-C₆alkyl groups contain 1 to 6 carbon atoms. Examples of a C₁-C₆alkyl group include, but are not limited to, methyl, ethyl, propyl, butyl, pentyl, isopropyl, isobutyl, sec-butyl and tert-butyl, isopentyl and neopentyl.

The term “alkenyl” means an aliphatic hydrocarbon group containing a carbon-carbon double bond and which may be straight or branched having about 2 to about 6 carbon atoms in the chain. Certain alkenyl groups have 2 to about 4 carbon atoms in the chain. Branched means that one or more lower alkyl groups such as methyl, ethyl, or propyl are attached to a linear alkenyl chain. Exemplary alkenyl groups include ethenyl, propenyl, n-butenyl, and z-butenyl. A C₂-C₆ alkenyl group is an alkenyl group containing between 2 and 6 carbon atoms.

The term “alkynyl” means an aliphatic hydrocarbon group containing a carbon-carbon triple bond and which may be straight or branched having about 2 to about 6 carbon atoms in the chain. Certain alkynyl groups have 2 to about 4 carbon atoms in the chain. Branched means that one or more lower alkyl groups such as methyl, ethyl, or propyl are attached to a linear alkynyl chain. Exemplary alkynyl groups include ethynyl, propynyl, n-butynyl, 2-butynyl, 3-methylbutynyl, and n-pentynyl. A C₂-C₆ alkynyl group is an alkynyl group containing between 2 and 6 carbon atoms.

The term “cycloalkyl” means monocyclic or polycyclic saturated carbon rings containing 3-18 carbon atoms. Examples of cycloalkyl groups include, without limitations, cyclopropyl, cyclobutyl, cyclopentyl, cyclohexyl, cycloheptanyl, cyclooctanyl, norboranyl, norborenyl, bicyclo[2.2.2]octanyl, or bicyclo[2.2.2]octenyl. A C₃-C₈ cycloalkyl is a cycloalkyl group containing between 3 and 8 carbon atoms. A cycloalkyl group can be fused (e.g., decalin) or bridged (e.g., norbornane).

The term “cycloalkenyl” means monocyclic, non-aromatic unsaturated carbon rings containing 4-18 carbon atoms. Examples of cycloalkenyl groups include, without limitation, cyclopentenyl, cyclohexenyl, cycloheptenyl, cyclooctenyl, and norborenyl. A C₄-C₈ cycloalkenyl is a cycloalkenyl group containing between 4 and 8 carbon atoms.

In some embodiments, the terms “heterocyclyl” or “heterocycloalkyl” or “heterocycle” refer to monocyclic or polycyclic 3 to 24-membered rings containing carbon and heteroatoms selected from oxygen, phosphorus, nitrogen, and sulfur and wherein there are no delocalized π electrons (aromaticity) shared among the ring carbon or heteroatoms. Heterocyclyl rings include, but are not limited to, oxetanyl, azetidinyl, tetrahydrofuranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, and homotropanyl. A heteroycyclyl or heterocycloalkyl ring can also be fused or bridged, e.g., can be a bicyclic ring.

In some embodiments “heterocyclyl” or “heterocycloalkyl” or “heterocycle” is a saturated, partially saturated or unsaturated, mono or bicyclic ring containing 3-24 atoms of which at least one atom is chosen from nitrogen, sulfur or oxygen, which may, unless otherwise specified, be carbon or nitrogen linked, wherein a —CH₂— group can optionally be replaced by a —C(O)— or a ring sulfur atom may be optionally oxidised to form the S-oxides. “Heterocyclyl” can be a saturated, partially saturated or unsaturated, mono or bicyclic ring containing 5 or 6 atoms of which at least one atom is chosen from nitrogen, sulfur or oxygen, which may, unless otherwise specified, be carbon or nitrogen linked, wherein a —CH₂— group can optionally be replaced by a —C(O)— or a ring sulfur atom may be optionally oxidised to form S-oxide(s). Non-limiting examples and suitable values of the term “heterocyclyl” are thiazolidinyl, pyrrolidinyl, pyrrolinyl, 2-pyrrolidonyl, 2,5-dioxopyrrolidinyl, 2-benzoxazolinonyl, 1,1-dioxotetrahydro thienyl, 2,4-dioxoimidazolidinyl, 2-oxo-1,3,4-(4-triazolinyl), 2-oxazolidinonyl, 5,6-dihydro uracilyl, 1,3-benzodioxolyl, 1,2,4-oxadiazolyl, 2-azabicyclo[2.2.1]heptyl, 4-thiazolidonyl, morpholino, 2-oxotetrahydrofuranyl, tetrahydrofuranyl, 2,3-dihydrobenzofuranyl, benzothienyl, tetrahydropyranyl, piperidyl, 1-oxo-1,3-dihydroisoindolyl, piperazinyl, thiomorpholino, 1,1-dioxothiomorpholino, tetrahydropyranyl, 1,3-dioxolanyl, homopiperazinyl, thienyl, isoxazolyl, imidazolyl, pyrrolyl, thiadiazolyl, isothiazolyl, 1,2,4-triazolyl, 1,3,4-triazolyl, pyranyl, indolyl, pyrimidyl, thiazolyl, pyrazinyl, pyridazinyl, pyridyl, 4-pyridonyl, quinolyl and 1-isoquinolonyl.

As used herein, the term “halo” or “halogen” means a fluoro, chloro, bromo, or iodo group.

The term “carbonyl” refers to a functional group comprising a carbon atom double-bonded to an oxygen atom. It can be abbreviated herein as “oxo,” as C(O), or as C═O.

“Spirocycle” or “spirocyclic” means carbogenic bicyclic ring systems with both rings connected through a single atom. The ring can be different in size and nature, or identical in size and nature. Examples include spiropentane, spirohexane, spiroheptane, spirooctane, spirononane, or spirodecane. One or both of the rings in a spirocycle can be fused to another carbocyclic, heterocyclic, aromatic, or heteroaromatic ring. One or more of the carbon atoms in the spirocycle can be substituted with a heteroatom (e.g., O, N, S, or P). A C₅-C₁₂ spirocycle is a spirocycle containing between 5 and 12 carbon atoms. In some embodiments, a C₅-C₁₂ spirocycle is a spirocycle containing from 5 to 12 carbon atoms. One or more of the carbon atoms can be substituted with a heteroatom.

The term “spirocyclic heterocycle,” “spiroheterocyclyl,” or “spiroheterocycle” is understood to mean a spirocycle wherein at least one of the rings is a heterocycle (e.g., at least one of the rings is furanyl, morpholinyl, or piperadinyl). A spirocyclic heterocycle can contain between 5 and 12 atoms, at least one of which is a heteroatom selected from N, O, S and P. In some embodiments, a spirocyclic heterocycle can contain from 5 to 12 atoms, at least one of which is a heteroatom selected from N, O, S and P.

The term “tautomers” refers to a set of compounds that have the same number and type of atoms, but differ in bond connectivity and are in equilibrium with one another. A “tautomer” is a single member of this set of compounds. Typically a single tautomer is drawn but it is understood that this single structure is meant to represent all possible tautomers that might exist. Examples include enol-ketone tautomerism. When a ketone is drawn it is understood that both the enol and ketone forms are part of the disclosure.

The SHP2 inhibitor may be administered alone as a monotherapy or in combination with one or more other therapeutic agent (e.g., an inhibitor of a MAP kinase pathway or an anti-cancer therapeutic agent) as a combination therapy. The SHP2 inhibitor may be administered as a pharmaceutical composition. The SHP2 inhibitor may be administered before, after, and/or concurrently with the one or more other therapeutic agent (e.g., an inhibitor of a MAP kinase pathway or an anti-cancer therapeutic agent). If administered concurrently with the one or more other therapeutic agent, such administration may be simultaneous (e.g., in a single composition) or may be via two or more separate compositions, optionally via the same or different modes of administration (e.g., local, systemic, oral, intravenous, etc.). In some embodiments, the SHP2 inhibitor may be administered in combination with a cancer immunotherapy, radiation therapy, and/or with surgical tumor resection and additionally or alternatively with one or more other therapeutic agent (e.g., an inhibitor of a MAP kinase pathway or an anti-cancer therapeutic agent).

Administration of the disclosed compositions and compounds (e.g., SHP2 inhibitors and/or other therapeutic agents) can be accomplished via any mode of administration for therapeutic agents. These modes include systemic or local administration such as oral, nasal, parenteral, transdermal, subcutaneous, vaginal, buccal, rectal or topical administration modes.

Depending on the intended mode of administration, the disclosed compounds or pharmaceutical compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, and all using forms well known to those skilled in the pharmaceutical arts. Pharmaceutical compositions suitable for the delivery of a SHP2 inhibitor (alone or, e.g., in combination with another therapeutic agent according to the present disclosure) and methods for their preparation will be readily apparent to those skilled in the art. Such compositions and methods for their preparation may be found, e.g., in Remington's Pharmaceutical Sciences, 19th Edition (Mack Publishing Company, 1995), incorporated herein in its entirety.

Illustrative pharmaceutical compositions are tablets and gelatin capsules comprising a SHP2 inhibitor alone or in combination with another therapeutic agent according to the disclosure and a pharmaceutically acceptable carrier, such as: a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algiic acid or its sodium salt, or effervescent mixtures; e) absorbent, colorant, flavorant and sweetener; f) an emulsifier or dispersing agent, such as Tween® 80, Labrasol®, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, a SHP2 inhibitor (alone or in combination with another therapeutic agent according to the disclosure) is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the SHP2 inhibitor (alone or in combination with another therapeutic agent according to the disclosure).

The SHP2 inhibitor can be also formulated as a suppository, alone or in combination with another therapeutic agent according to the disclosure, which can be prepared from fatty emulsions or suspensions; using polyalkylene glycols such as propylene glycol, as the carrier.

The SHP2 inhibitor can also be administered in the form of liposome delivery systems, such as small unilamellar vesicles, large unilamellar vesicles and multilamellar vesicles, either alone or in combination with another therapeutic agent according to the disclosure. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described for instance in U.S. Pat. No. 5,262,564, the contents of which are hereby incorporated by reference.

SHP2 inhibitors can also be delivered by the use of monoclonal antibodies as individual carriers to which the disclosed compounds are coupled. SHP2 inhibitors can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, a SHP2 inhibitor can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels. In one embodiment, disclosed compounds are not covalently bound to a polymer, e.g., a polycarboxylic acid polymer, or a polyacrylate.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

Another aspect of the invention relates to a pharmaceutical composition comprising a SHP2 inhibitor (alone or in combination with another therapeutic agent according to the present disclosure) and a pharmaceutically acceptable carrier. The pharmaceutically acceptable carrier can further include an excipient, diluent, or surfactant.

Thus, the present disclosure provides compositions (e.g., pharmaceutical compositions) comprising one or more SHP2 inhibitor for use in a method disclosed herein, e.g., a SHP2 monotherapy. Such compositions may comprise a SHP2 inhibitor and, e.g., one or more carrier, excipient, diluent, and/or surfactant.

The present disclosure provides compositions (e.g., pharmaceutical compositions) comprising one or more SHP2 inhibitor and one or more additional therapeutic agent for use in a method disclosed herein, e.g., a SHP2 combination therapy. Such compositions may comprise a SHP2 inhibitor, an additional therapeutic agent (e.g., a TKI, a MAPK pathway inhibitor, an EGFR inhibitor, an ALK inhibitor, a MEK inhibitor) and, e.g., one or more carrier, excipient, diluent, and/or surfactant.

The present disclosure provides compositions (e.g., pharmaceutical compositions) comprising one or more SHP2 inhibitors and one or more MEK inhibitors for use in a method disclosed herein, e.g., a SHP2 combination therapy. Such compositions may comprise a SHP2 inhibitor, a MEK inhibitor and, e.g., one or more carrier, excipient, diluent, and/or surfactant. Such compositions may consist essentially of a SHP2 inhibitor, a MEK inhibitor and, e.g., one or more carrier, excipient, diluent, and/or surfactant. Such compositions may consist of a SHP2 inhibitor, a MEK inhibitor and, e.g., one or more carrier, excipient, diluent, and/or surfactant. For example, one non-limiting example of a composition of the present disclosure may comprise, consist essentially of, or consist of (a) a SHP2 inhibitor; (b) a MEK inhibitor selected from one or more of Trametinib (GSK1120212); Selumetinib (AZD6244); Cobimetinib (GDC-0973/XL581), Binimetinib, Vemurafenib, Pimasertib, TAK733, RO4987655 (CH4987655); CI-1040; PD-0325901; Refametinib (RDEA 119/BAY 86-9766); RO5126766, AZD8330 (ARRY-424704/ARRY-704); and GSK1120212; and (c) one or more carrier, excipient, diluent, and/or surfactant. Another non-limiting example of a composition of the present disclosure may comprise, consist essentially of, or consist of (a) a MEK inhibitor; (b) a SHP2 inhibitor selected from (i) RMC-3943; (ii) RMC-4550; (iii) Compound C; (iv) SHP099; (v) a SHP2 inhibitor compound of any one of Formula I, of Formula II, of Formula III, of Formula I-V1, of Formula I-V2, of Formula I-W, of Formula I-X, of Formula I-Y, of Formula I-Z, of Formula IV, of Formula V, of Formula VI, of Formula IV-X, of Formula IV-Y, of Formula IV-Z, of Formula VII, of Formula VIII, of Formula IX, and of Formula X disclosed herein; (vi) TN0155; (vii) a compound from Table 1, disclosed herein; (viii) a compound from Table 2, disclosed herein, and (xi) a combination thereof; and (c) one or more carrier, excipient, diluent, and/or surfactant.

Compositions can be prepared according to conventional mixing, granulating or coating methods, respectively, and the present pharmaceutical compositions can contain from about 0.1% to about 99%, from about 5% to about 90%, or from about 1% to about 20% of the disclosed therapeutic agent by weight or volume. Accordingly, such compositions may contain from about 0.1% to about 99%, from about 5% to about 90%, or from about 1% to about 20% of the disclosed Compound C by weight or volume. Compositions may contain from about 0.1% to about 99%, from about 5% to about 90%, or from about 1% to about 20% of the disclosed RMC-4550 by weight or volume. Compositions may contain from about 0.1% to about 99%, from about 5% to about 90%, or from about 1% to about 20% of a SHP2 inhibitor compound listed in Table 1 by weight or volume. Compositions may contain from about 0.1% to about 99%, from about 5% to about 90%, or from about 1% to about 20% of a SHP2 inhibitor compound listed in Table 2 by weight or volume. Compositions may contain from about 0.1% to about 99%, from about 5% to about 90%, or from about 1% to about 20% of a combination of two or more SHP2 inhibitors by weight or volume, e.g., of Compound C and one or more additional SHP2 inhibitor by weight or by volume.

The dosage regimen utilizing the disclosed compound is selected in accordance with a variety of factors including type, species, age, weight, sex and medical condition of the patient; the severity of the condition to be treated; the route of administration; the renal or hepatic function of the patient; and the particular disclosed compound employed. A physician or veterinarian of ordinary skill in the art can readily determine and prescribe the effective amount of the drug required to prevent, counter or arrest the progress of the condition.

Effective dosage amounts of a SHP2 inhibitor, when used for the indicated effects, range from about 0.5 mg to about 5000 mg as needed to treat the condition. Compositions for in vivo or in vitro use can contain about 0.5, 5, 20, 50, 75, 100, 150, 250, 500, 750, 1000, 1250, 2500, 3500, or 5000 mg of the disclosed compound, or, in a range of from one amount to another amount in the list of doses. In one embodiment, the compositions are in the form of a tablet that can be scored.

The present invention also provides kits for treating a disease or disorder with a SHP2 inhibitor, one or more carrier, excipient, diluent, and/or surfactant, and a means for determining whether a sample from a subject (e.g., a tumor sample) is likely to be sensitive to SHP2 treatment. In some embodiments, the means for determining comprises a means for determining whether the sample comprises an RTK fusion. In some embodiments, the means for determining comprises a means for determining whether the sample comprises and RTK fusion that activates the MAPK pathway. In some embodiments, the means for determining comprises a means for determining whether the sample comprises any of the RTK fusion mutations described herein. Such means include, but are not limited to direct sequencing, and utilization of a high-sensitivity diagnostic assay (with CE-IVD mark), e.g., as described in Domagala, et al., Pol J Pathol 3: 145-164 (2012), incorporated herein by reference in its entirety, including TheraScreen® PCR; AmoyDx; PNAClamp; RealQuality; EntroGen; LightMix; StripAssay®; Hybcell plexA; Devyser; Surveyor; Cobas; and TheraScreen Pyro. In some embodiments, the means for determining comprises a means for determining whether a sample that comprises an RTK fusion mutations described herein activates the MAPK pathway. Thus, the means may be an immunoblot; immunofluorescence; or ELISA.

All of the U.S. patents, U.S. patent application publications, U.S. patent applications, PCT patent application, PCT patent application publications, foreign patents, foreign patent applications and non-patent publications referred to in this specification or listed in any Application Data Sheet are incorporated herein by reference in their entirety. From the foregoing it will be appreciated that, although specific embodiments of the invention have been described herein for purposes of illustration, various modifications may be made without deviating from the spirit and scope of the invention.

### Example Embodiments

Some embodiments of this disclosure are Embodiment I, as follows:

Embodiment I-1. A method for identifying whether a subject has a cancer that is sensitive to SHP2 inhibition, the method comprising determining whether the cancer comprises one or more cells containing an oncogenic tyrosine kinase fusion that causes MAPK activation, and, if so, identifying the subject as having a cancer that is sensitive to SHP2 inhibition.

Embodiment I-1a. A SHP2 inhibitor for use in a method for treating a subject having a cancer, wherein the cancer comprises a cell containing an oncogenic tyrosine kinase fusion that causes MAPK activation.

Embodiment I-1b. A method of selecting a subject having a cancer for treatment with a SHP2 inhibitor,
wherein the method comprises determining in vitro whether the cancer comprises one or more cells containing an oncogenic tyrosine kinase fusion that causes MAPK activation; and wherein the subject is selected for treatment with the SHP2 inhibitor if the biological sample contains an oncogenic tyrosine kinase fusion that causes MAPK activation.

Embodiment I-1c. A SHP2 inhibitor for use in a method for treating a subject having a cancer, wherein the method comprises
determining in vitro whether the cancer comprises one or more cells containing an oncogenic tyrosine kinase fusion that causes MAPK activation; and
administering to the subject the SHP2 inhibitor if the cancer comprises one or more cells containing an oncogenic tyrosine kinase fusion that causes MAPK activation.

Embodiment I-2. A method for using a SHP2 inhibitor to treat a subject with a cancer, the method comprising the steps of:
determining whether the cancer comprises one or more cells that contain an oncogenic tyrosine kinase fusion that causes MAPK activation; and
administering the SHP2 inhibitor to the patient if the cancer comprises a cell that contains an oncogenic tyrosine kinase fusion that causes MAPK activation.

Embodiment I-3. A method for killing cancer cells with a SHP2 inhibitor, the method comprising the steps of:
determining whether one or more of the cancer cells contain an oncogenic tyrosine kinase fusion that causes MAPK activation; and
contacting the cancer cells with the SHP2 inhibitor if one or more of the cancer cells contains an oncogenic tyrosine kinase fusion that causes MAPK activation.

Embodiment I-3a. A SHP2 inhibitor for use in a method for killing cancer cells, wherein one or more of the cancer cells contains an oncogenic tyrosine kinase fusion that causes MAPK activation.

Embodiment I-3b. Use of a SHP2 inhibitor for the manufacture of a medicament for killing a cell containing an oncogenic tyrosine kinase fusion that causes MAPK activation.

Embodiment I-3c. A SHP2 inhibitor for use in a method for killing cancer cells, wherein the method comprises
determining in vitro whether one or more of the cancer cells contains an oncogenic tyrosine kinase fusion that causes MAPK activation; and
contacting the cancer cells with the SHP2 inhibitor if one or more of the cancer cells contains an oncogenic tyrosine kinase fusion that causes MAPK activation.

Embodiment I-4. A method for treating a patient with a SHP2 inhibitor, wherein the patient has cancer, the method comprising the steps of:
determining whether the patient has a SHP2-sensitive cancer by:
obtaining or having obtained a biological sample from the patient; and
performing or having performed an assay on the biological sample to determine if the patient has a tumor comprising one or more cells that contain an oncogenic tyrosine kinase fusion that causes MAPK activation; and
administering the SHP2 inhibitor to the patient if the patient has a tumor comprising one or more cells containing an oncogenic tyrosine kinase fusion that causes MAPK activation.

Embodiment I-4b. A method of selecting a patient that has cancer for treatment with a SHP2 inhibitor;
wherein the method comprises determining in vitro whether the patient has a SHP2-sensitive cancer by:
obtaining or having obtained a biological sample from a patient; and
performing or having performed an in vitro assay on the biological sample to determine if the biological sample comprises one or more cells containing an oncogenic tyrosine kinase fusion that causes MAPK activation; and
wherein the patient is selected for treatment with the SHP2 inhibitor if the biological sample comprises one or more cells containing an oncogenic tyrosine kinase fusion that causes MAPK activation.

Embodiment I-4c. A SHP2 inhibitor for use in a method for treating a patient that has cancer, wherein the method comprises the steps of:
determining in vitro whether the patient has a SHP2-sensitive cancer by:
obtaining or having obtained a biological sample from a patient; and
performing or having performed an in vitro assay on the biological sample to determine if the sample comprises one or more cells containing an oncogenic tyrosine kinase fusion that causes MAPK activation; and
administering the SHP2 inhibitor to the patient if the biological sample comprises one or more cells containing an oncogenic tyrosine kinase fusion that causes MAPK activation.

Embodiment I-5. The method of any one of Embodiments I-1, I-1a, I-1b, I-1c, I-2, I-3, I-3a, I-3b, I-3c, I-4, I-4b, and I-4c, wherein the SHP2 inhibitor is selected from (i) NSC-87877; (ii) TN0155, (iii) of any one of Formula I, of Formula II, of Formula III, of Formula I-V1, of Formula I-V2, of Formula I-W, of Formula I-X, of Formula I-Y, of Formula I-Z, of Formula IV, of Formula V, of Formula VI, of Formula IV-X, of Formula IV-Y, of Formula IV-Z, of Formula VII, of Formula VIII, of Formula IX, and of Formula X disclosed in PCT/US2017/041577 (WO 2018/013597), incorporated herein by reference in its entirety; (iv) Compound C; (v) a SHP2 inhibitor listed on Table 1; (vi) a SHP2 inhibitor listed on Table 2; and (vii) combinations thereof.

Embodiment I-5b. The method of any one of Embodiments I-1, I-1a, I-1b, I-1c, I-2, I-3, I-3a, I-3b, I-3c, I-4, I-4b, and I-4c, wherein the SHP2 inhibitor is a pharmaceutically acceptable salt, prodrug, solvate, hydrate, tautomer, or isomer of a SHP2 inhibitor selected from (i) NSC-87877; (ii) TN0155, (iii) of any one of Formula I, of Formula II, of Formula III, of Formula I-V1, of Formula I-V2, of Formula I-W, of Formula I-X, of Formula I-Y, of Formula I-Z, of Formula IV, of Formula V, of Formula VI, of Formula IV-X, of Formula IV-Y, of Formula IV-Z, of Formula VII, of Formula VIII, of Formula IX, and of Formula X d herein; (iv) Compound C; (v) a SHP2 inhibitor listed on Table 1; or (vi) a SHP2 inhibitor listed on Table 2; or a combination of any two or more of such pharmaceutically acceptable salts, prodrugs, solvates, hydrates, tautomers, or isomers.

Embodiment I-6. The method of any one of Embodiments I-1, I-1a, I-1b, I-1c, I-2, I-3, I-3a, I-3b, I-3c, I-4, I-4b, I-4c, I-5; and I5-b, wherein the oncogenic tyrosine kinase fusion is selected from a ROS1 fusions, an ALK fusion, a RET fusion, an NTRK1 fusion, NTRK2 fusion, and NTRK3 fusion.

Embodiment I-7. The method of any one of Embodiments I-1, I-1a, I-1b, I-1c, I-2, I-3, I-3a, I-3b, I-3c, I-4, I-4b, I-4c, I-5, I5-b, and 1-6, wherein the oncogenic tyrosine kinase fusion is a SDC4-ROS1 fusion or an SLC34A2-ROS1 fusion.

Embodiment I-8. The method of any one of Embodiments I-1, I-1a, I-1b, I-1c, I-2, I-3, I-3a, I-3b, I-3c, I-4, I-4b, I-4c, I-5, I5-b, and I-6, wherein the oncogenic tyrosine kinase fusion is selected from a FIG-ROS1 fusion; a LRIG3-ROS1 fusion; an EZR-ROS1 fusion, and a TPM3-ROS1 fusion.

Embodiment I-9. The method of any one of Embodiments I-1, I-1a, I-1b, I-1c, I-2, I-3, I-3a, I-3b, I-3c, I-4, I-4b, I-4c, I-5, I5-b, and I-6, wherein the oncogenic tyrosine kinase fusion is selected from an EML4-ALK fusion.

Embodiment I-10. The method of any one of Embodiments I-1, I-1a, I-1b, I-1c, I-2, I-3, I-3a, I-3b, I-3c, I-4, I-4b, I-4c, I-5, I5-b, I-6. I-7, I-8, and I-9, wherein the MAPK activation is detected by measuring increased ERK phosphorylation.

Embodiment I-11. The method of any one of Embodiments I-1, I-1a, I-1b, I-1c, I-2, I-3, I-3a, I-3b, I-3c, I-4, I-4b, I-4c, I-5, I5-b, I-6. I-7, I-8, I-9, and I-10, wherein determining whether the cancer cells contain an oncogenic tyrosine kinase fusion that causes MAPK activation is achieved by genotyping a cell or cells in a biological sample obtained from the patient.

Embodiment I-12. The method of Embodiments I-1, I-1a, I-1b, I-1c, I-2, I-3, I-3a, I-3b, I-3c, I-4, I-4b, I-4c, I-5, I5-b, I-6. I-7, I-8, I-9, I-10, and I-11, wherein the genotyping determines whether the cancer comprises a cell containing an oncogenic tyrosine kinase fusion selected from EML4-ALK, SDC4-ROS1 and SLC34A2-ROS1.

Embodiment I-13. The method of any one of Embodiments I-1, I-1a, I-1b, I-1c, I-2, I-3, I-3a, I-3b, I-3c, I-4, I-4b, I-4c, I-5, I5-b; I-6. I-7, I-8, I-9, I-10, I-11, and I-12, wherein if the cancer does not comprise any cells containing an oncogenic tyrosine kinase fusion that causes MAPK activation, then the method comprises administering a cancer therapy selected from chemotherapy, radiation therapy, and/or surgical tumor resection.

Embodiment I-14. A method for treating a subject having a tumor with a SHP2 inhibitor, the method comprising:
determining whether a biological sample obtained from the subject contains an oncogenic tyrosine kinase fusion protein comprising a N-terminal fusion partner that causes the fusion protein to localize in an endosome; and
administering to the subject the SHP2 inhibitor if the biological sample contains an oncogenic tyrosine kinase fusion protein comprising a N-terminal fusion partner that causes the fusion protein to localize in an endosome.

Embodiment I-15. The method of Embodiment I-14, wherein the oncogenic tyrosine kinase fusion protein causes MAPK activation.

Embodiment I-16. The method of any one of Embodiments I-1, I-1c, I-2, I-4, I-4c, I-5, I5-b; I-6, I-7, I-8, I-9, I-10, I-11, I-12, I-14, and I-15, wherein the method further comprises administering a cancer therapy selected from chemotherapy, radiation therapy, and/or surgical tumor resection.

Embodiment I-17. The method of any one of Embodiments I-1, I-1c, I-2, I-4, I-4c, I-5, I5-b; I-6, I-7, I-8, I-9, I-10, I-11, I-12, I-14, I-15 and I-16, wherein the method further comprises administering an additional therapeutic agent (e.g., a TKI, a MAPK pathway inhibitor, an EGFR inhibitor, an ALK inhibitor, a MEK inhibitor.

Embodiment I-18. The method of Embodiment I-3, wherein the contacting occurs in vivo in a subject.

Embodiment I-19. The method of Embodiment I-18, wherein the contacting occurs via administration of the SHP2 inhibitor to the subject.

Embodiment I-20. The method of Embodiment I-19, wherein the method further comprises administering a cancer therapy selected from chemotherapy, radiation therapy, and/or surgical tumor resection.

Embodiment I-21. The method of Embodiment I-19 or I-20, wherein the method further comprises administering an additional therapeutic agent (e.g., a TKI, a MAPK pathway inhibitor, an EGFR inhibitor, an ALK inhibitor, a MEK inhibitor.

Embodiment I-22. The method of Embodiment I-19 or I-20, wherein the method further comprises administering an additional therapeutic agent, wherein the additional therapeutic agent is (i) a pharmaceutically acceptable salt, prodrug, solvate, hydrate, tautomer, or isomer of a TKI, a MAPK pathway inhibitor, an EGFR inhibitor, an ALK inhibitor, or a MEK inhibitor, or (ii) a combination of any two or more of such pharmaceutically acceptable salts, prodrugs, solvates, hydrates, tautomers, or isomers recited in (i).

Embodiment I-22. The method of any one of Embodiments I-1a, I-1b, I-1c, I-2, I-3. I-3a, I-3b, I-3c, I-4, I-4b, I-4c, I-5,15-b, I-6, I-7, I-8, I-9, I-10, I-11, I-12, I-13, I-14, I-15, I-16. I-17, I-18, I-19, I-20, and I-21, wherein the SHP2 inhibitor is Compound C.

Embodiment I-22b. The method of any one of Embodiments I-1a, I-1b, I-1c, I-2, I-3. I-3a, I-3b, I-3c, I-4, I-4b, I-4c, I-5,15-b, I-6, I-7, I-8, I-9, I-10, I-11, I-12, I-13, I-14, I-15, I-16. I-17, I-18, I-19, I-20, and I-21, wherein the SHP2 inhibitor is a pharmaceutically acceptable salt, prodrug, solvate, hydrate, tautomer, or isomer of Compound C.

Embodiment I-23. The method of any one of Embodiments I-1a, I-1b, I-1c, I-2, I-3. I-3a, I-3b, I-3c, I-4, I-4b, I-4c, I-5,15-b, I-6, I-7, I-8, I-9, I-10, I-11, I-12, I-13, I-14, I-15, I-16, I-17, I-18, I-19, I-20, and I-21, wherein the SHP2 inhibitor is selected from the group of SHP2 inhibitors consisting of:

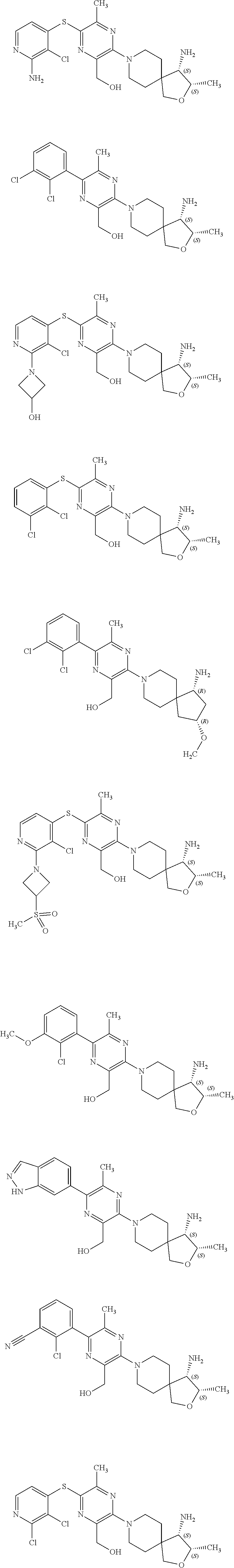

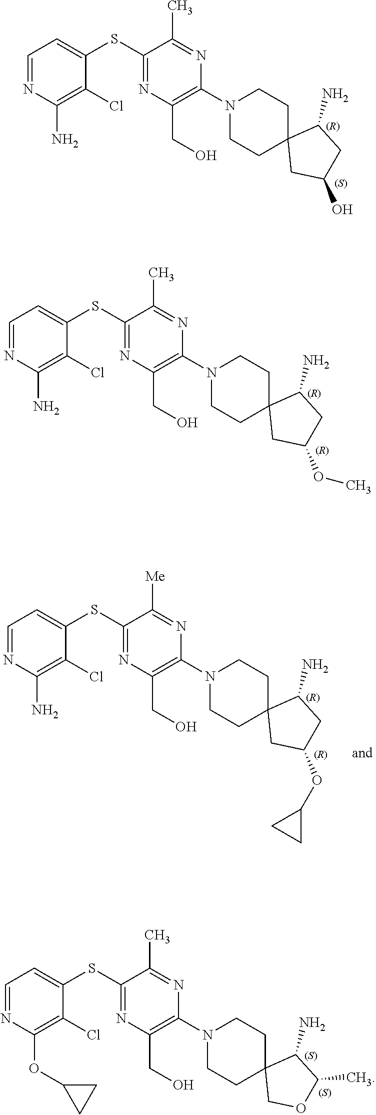

Embodiment I-23b. The method of any one of Embodiments I-1a, I-1b, I-1c, I-2, I-3. I-3a, I-3b, I-3c, I-4, I-4b, I-4c, I-5,15-b, I-6, I-7, I-8, I-9, I-10, I-11, I-12, I-13, I-14, I-15, I-16. I-17, I-18, I-19, I-20, and I-21, wherein the SHP2 inhibitor is a pharmaceutically acceptable salt, prodrug, solvate, hydrate, tautomer, or isomer of a compound selected from the group of SHP2 inhibitors consisting of:

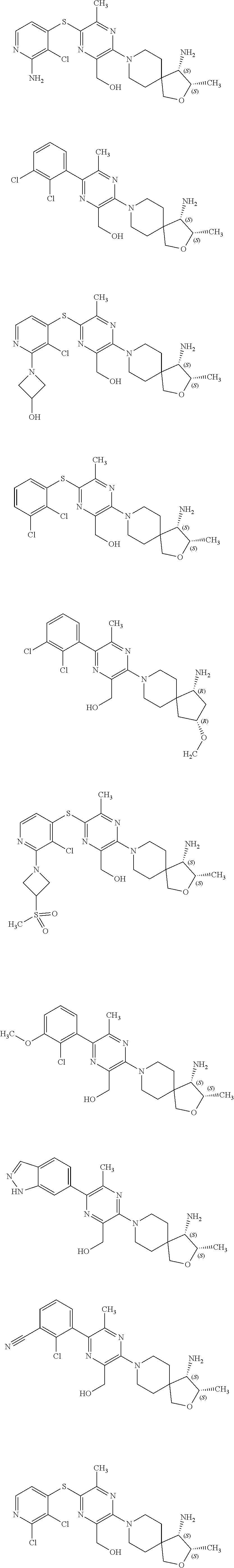

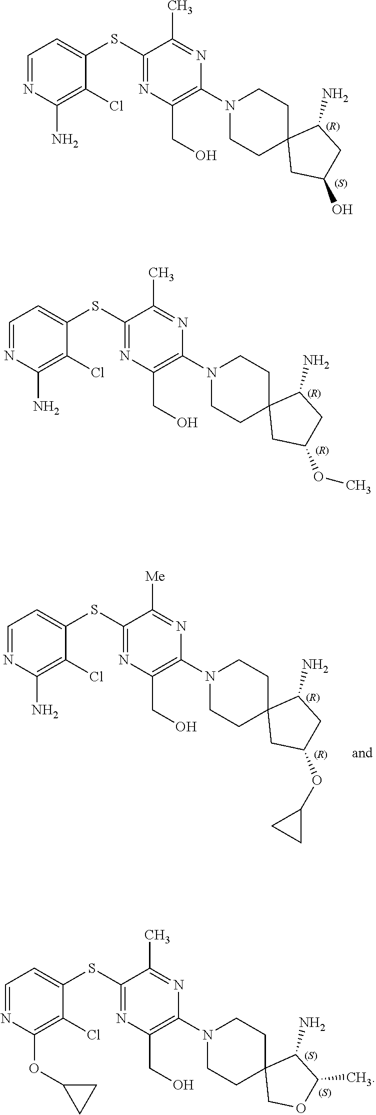

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1

### Materials & Methods

Unless otherwise stated, the Examples disclosed herein utilize the following materials and methods.

Cell Culture.

All cell lines were maintained in a humidified incubator at 37° C., 5% CO₂. The patient-derived ROS1-positive lung adenocarcinoma lines HCC78, CUTO-2, CUTO-23, and CUTO-33, and the normal lung epithelial line BEAS2-B were all maintained in RPMI-1640 supplemented with 10% FBS and 100 ug/mL of penicillin/streptomycin. HEK-293T cells and NIH-3T3 cells were maintained in DMEM-High Glucose supplemented with 10% FBS and 100 ug/mL of penicillin/streptomycin. CUTO-2, CUTO-23, and CUTO-33 cells were a generous gift from Dr. Robert Doebele (University of Colorado, Denver, Colo., USA).

Compounds. Crizotinib (Selleck Chemicals, Houston, Tex., USA) and the SHP2 inhibitors RMC-4550 (Revolution Medicines, Redwood City, Calif., USA), Compound C (Revolution Medicines, Redwood City, Calif., USA), and RMC-3943 (Revolution Medicines, Redwood City, Calif., USA) were dissolved in DMSO.

Antibodies. The following Cell Signaling Technology (Danvers, Mass., USA) antibodies were used: phospho-ROS1 (Y2274, #3078), ROS1 (#3287), phospho-ALK (Y1604, #3341), ALK (#3633), phospho-STAT3 (Y705, #9145), STAT3 (#9139), phospho-AKT (S473, #5012), AKT (#2920), phospho-ERK (Y202/204, #4370), ERK (#4694), phospho-MEK1/2 (Ser 217/221, #9121), MEK1 (#2352), Anti-rabbit IgG, HRP-linked Antibody (#7074), Anti-mouse IgG, HRP-linked Antibody (#7076). The following Sigma-Aldrich (St Louis, Mo., USA) antibodies were used: Beta-Actin (#A2228). The following Santa Cruz Biotechnology (Santa Cruz, Calif., USA) antibodies were used: EEA1 (sc-6415). The following Abeam (Cambridge, UK) antibodies were used: Calnexin-Alexa Fluor® 488 (ab202574), PTP1B (ab201974). The following Life Technologies Thermo Fisher Scientific (Waltham, Mass., USA) antibodies were used: Alexa Fluor® 488 Donkey Anti-Mouse (#21202), Alexa Fluor® 499 Donkey Anti-Goat (#11055), Alexa Fluor®594 Donkey Anti-Rabbit (#21207).

DNA Transfections.

293T cells were transiently transfected using TransIt®-LT1 transfection reagent (Mirus Bio LLC, Madison, Wis., USA).

Immunoblotting.

For immunoblotting, cells were washed with ice-cold PBS and scraped in ice-cold RIPA buffer [25 mM Tris-HCl (pH 7.6), 150 mM NaCl, 1% NP-40, 1% sodium deoxycholate, 0.1% SDS, supplemented with 1×HALT protease inhibitor cocktail and 1×HALT phosphatase inhibitor cocktail (Thermo Fisher Scientific, Waltham, Mass.)]. Lysates were clarified with sonication and centrifugation. Lysates were subject to SDS/PAGE followed by blotting with the indicated antibodies. Signal was detected using Amersham ECL Prime reagent (GE Healthcare Life Sciences) and chemiluminescnce on an ImageQuant LAS 4000 (GE Healthcare Life Science, Chicago, Ill., USA). 293T cells were serum starved (0% S) for 5 hours and ROS1 BEAS2-B cells were serum starved (0% S) for 24 hours prior to lysate collection.

siRNA Knockdown.

Cells were seeded in 6-well plates. The following day, siRNA were resuspended to a final concentration of 5 uM in serum-free medium with DharmaFECT transfection reagent (Thermo Fisher Scientific), then pipetted onto cells. Lysates were harvested 55 hours later. The following ROS1 siRNAs from Sigma-Aldrich were used: Hs01_00183685 (siROS1 #1) and Hs01_00183690 (siROS1 #2). Non-targeting control siRNA was purchased from Dharmacon (GE Life Sciences).

Constructs.

Lentiviral expression constructs for SDC4-ROS and CD74-ROS were generous gifts from Dr. Christine Lovly (Vanderbilt University, Nashville, Tenn., USA). Lentiviral expression construct for SLC34A2-ROS was a generous gift from Dr. Monika Davare (OHSU, Portland, Oreg., USA). The retroviral expression constructs for MEK-DD (#15268) and CA-STAT3 (#24983) were purchased on Addgene.

Viral Transduction.

293T viral packaging cells were plated in 10 cm dishes the day prior to transfection. They were transfected with lentiviral or retroviral expression constructs and the appropriate packaging plasmids using TransIt®-LT1 transfection reagent (Mirus Bio LLC, Madison, Wis., USA). Viral supernatants were collected 48-72 hours post-transfection and used to transduce cell lines in the presence of 1× Polybrene for 24 hours. 72 hours post-infection, media was changed to standard growth media plus the appropriate selectable marker (1 ug/mL puromycin for all lines except NIH-3T3, which were selected with 2 ug/mL puromycin). CA-STAT3-infected cells were sorted on a BD FACSAria II (BD Biosciences, San Jose, Calif.) for GFP-positivity.

Crystal Violet Assays.

Cells were seeded in 12-well plates at 10% confluency and treated with drug the following day. They were grown for 6-8 days, then fixed with 4% paraformaldehyde and stained with crystal violet. Pictures of stained cells were taking using transillumination on an ImageQuant LAS 4000 (GE Healthcare, Chicago, Ill., USA). Crystal violet was dissolved in 500 ul 1% SDS and quantified based on 470 nM absorbance using a SpectraMax spectrophotomer (Molecular Devices, Sunnyvale, Calif., USA). Relative cell viability was determined by normalizing to DMSO-treated control. All crystal violet images are representative and quantification values arise from ≥n=3 experiments. Statistical significance was determined by multiple t-test analysis using Prism 6 (Graphpad Software, La Jolla, Calif., USA).

Immunofluorescence.

Cells were seeded in 4-well Lab Tek® II Chamber Slides (Thermo Fisher Scientific). The following day, cells were fixed for 15 minutes with 4% paraformaldehyde, washed, and incubated in blocking buffer for 1 hour (1×PBS with 1% BSA and 0.3% Triton X-100). Blocking buffer was aspirated and cells were incubated with primary antibody overnight in the dark at 4° C. The following day, cells were washed, incubated with fluorophore-conjugated secondary antibody for 1 hour at room temperature in the dark, washed, then mounted using ProLong® Gold Antifade reagent with DAPI (Cell Signaling Technology, Danvers, Mass.). Slides were analyzed using a Nikon Ti microscope with a CSU-W1 spinning disk confocal (Nikon Imaging Center, UCSF).

Xenografts.

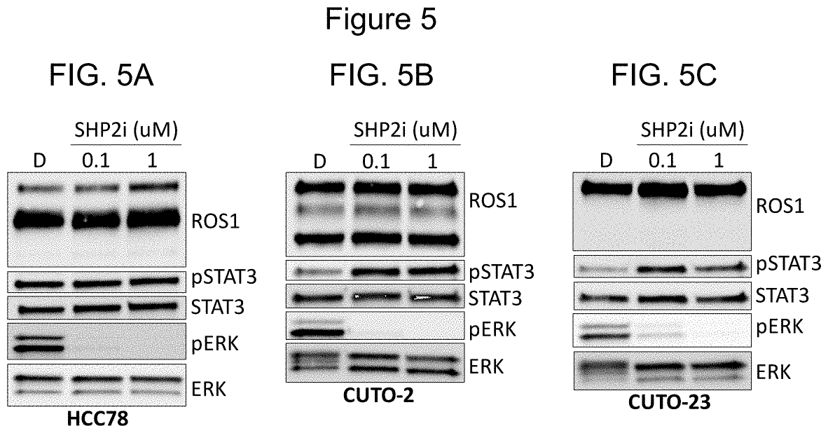

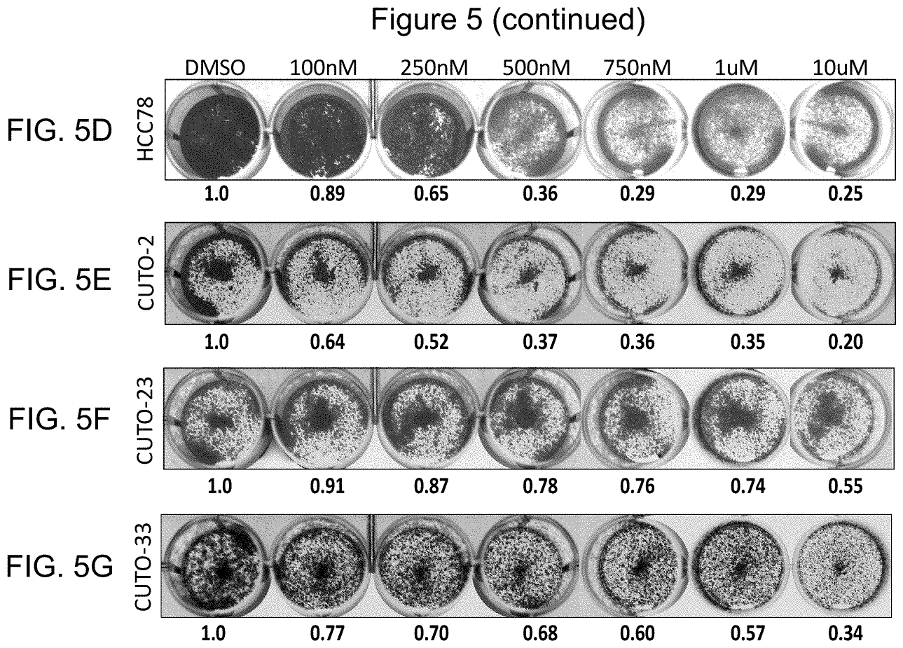

NIH-3T3 xenografts for were generated by injecting 1×10⁶ cells (FIG. 5b) or 5×10⁵ cells (FIG. 5c) in matrigel in flanks of 8-week old NOD/SCID mice. Mice were randomized to treatment groups once tumors reached a size of 150 mm³ (n=6 tumors per treatment group).

### Example 2

### ROS1 Fusion Oncoproteins Differentially Activate the RAS/MAPK Pathway

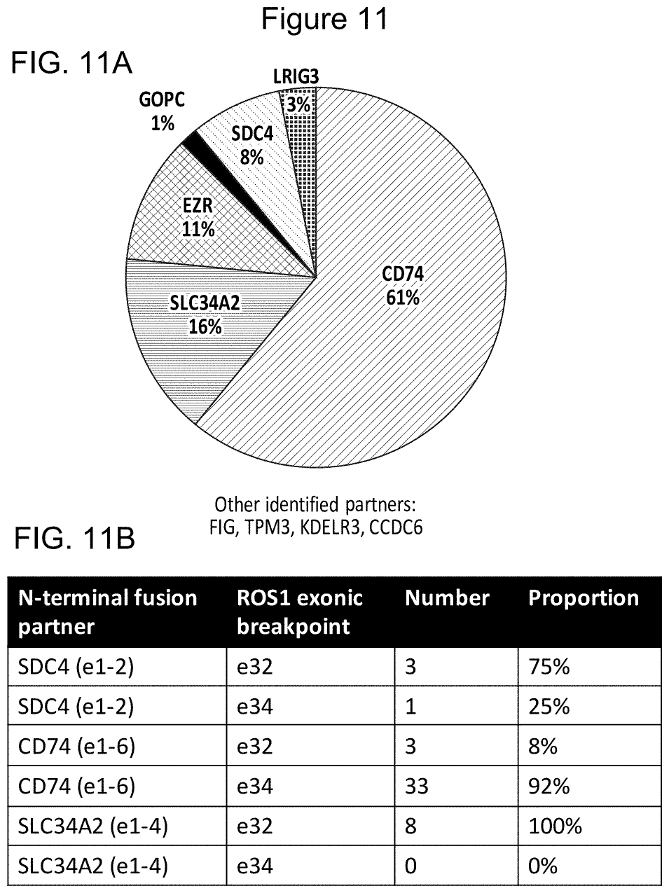

Fusions involving the RTK ROS1 are found in 1-2% of lung adenocarcinomas. (Bergethon et al., 2012; Takeuchi et al., 2012) ROS1 is one of the last remaining orphan receptor tyrosine kinases, and little is known about the wildtype function of the protein. Wildtype ROS1 contains a substantial N-terminal extracellular domain, whose structure suggests extracellular matrix proteins may serve as ligands. (Acquaviva et al., 2009) In cancer-driving ROS1 gene fusions this extracellular domain is not included, leaving the transmembrane and entire kinase domain of ROS1 fused to a variety of N-terminal fusion partners. (Davies and Doebele, 2013; Takeuchi et al., 2012) To date, 10 distinct N-terminal fusion partners for ROS1 kinase fusions have been identified in cancers (FIG. 11). (Forbes et al., 2017) The most common ROS1 fusion partner is CD74 (found in ˜50% of ROS1 fusions). (Kohno et al., 2015) Other commonly observed ROS1 fusion partners include SDC4, SLC34A2, LRIG3, EZR, and TPM3. (Davies and Doebele, 2013; Govindan et al., 2012; Seo et al., 2012) All of these N-terminal partners lack clearly unifying protein domains or functions, raising the possibility that not all of these fusion proteins promote oncogenic signaling and cancer growth identically. Whether the N-terminal partner in ROS1 oncoprotein fusions regulates the subcellular localization and oncogenic properties of each kinase fusion or response to TKI treatment is not well-understood.

We tested the hypothesis that that different ROS1 oncoprotein fusions engage distinct downstream signaling pathways and exhibit different oncogenic properties, and investigated the mechanistic role of specific N-terminal fusion partners in regulating such differential phenotypes through differential subcellular localization.

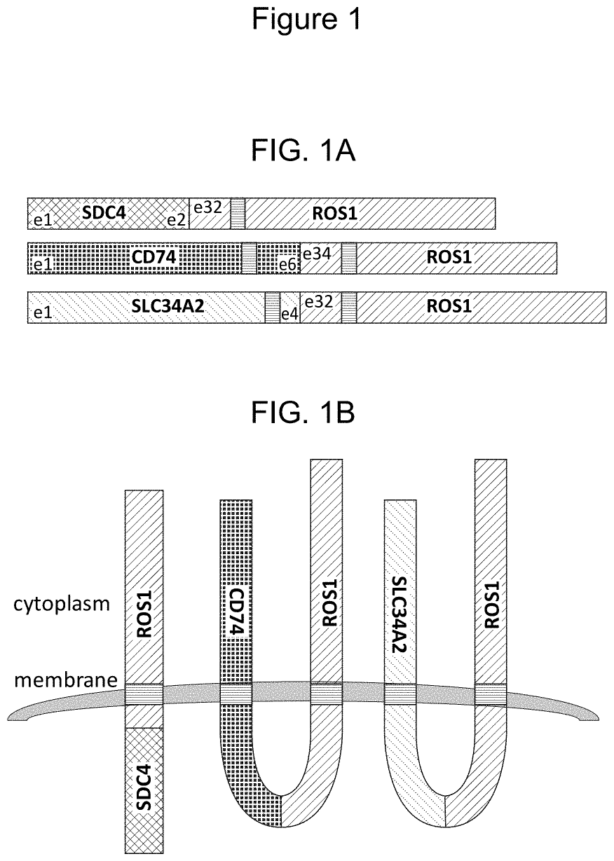

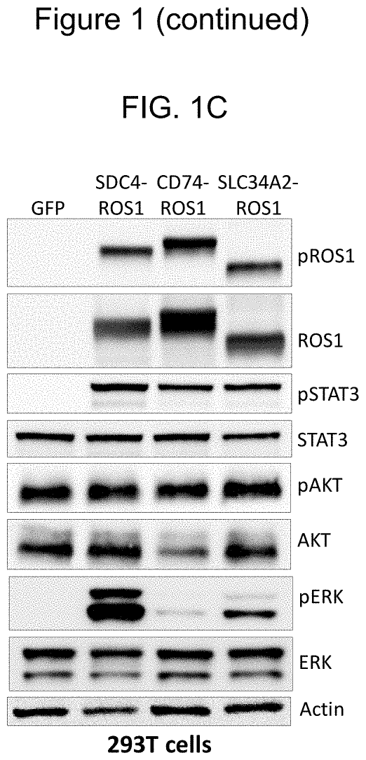

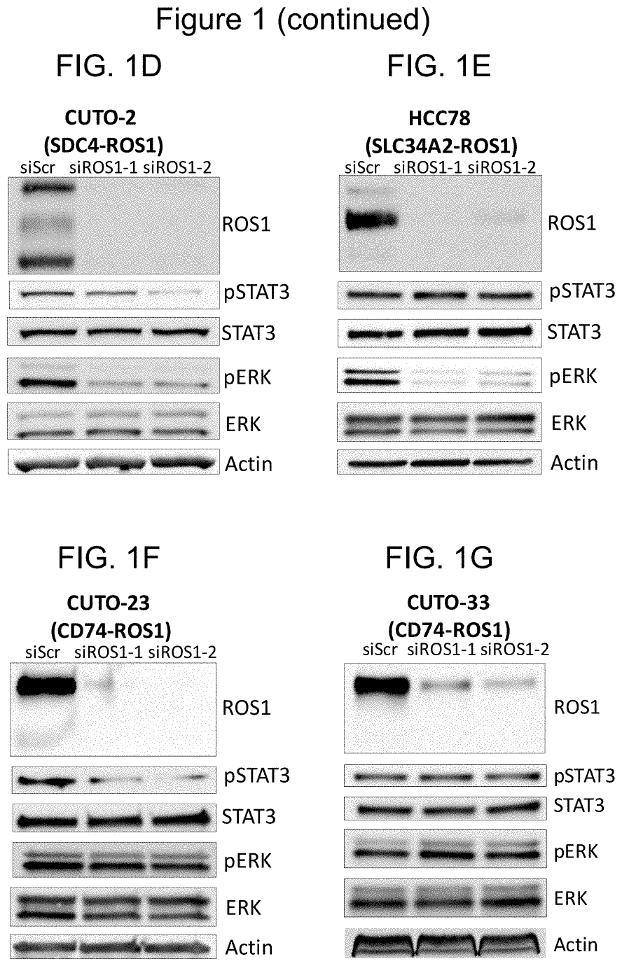

To investigate the potential differential functional properties of differential ROS1 oncoprotein fusions, first we engineered a genetically-controlled isogenic system to express some of the most common forms of ROS1 fusion oncoproteins that are present in patient tumors, including CD74-ROS1, SDC4-ROS1, and SLC34A2-ROS1 (FIG. 1A). (Forbes et al., 2017) Using an established predictive computational analysis, we determined that all of these fusions were topologically predicted to result in a cytoplasmic-facing kinase domain (FIG. 1B). (Dobson et al., 2015a; 2015b) All three ROS1 fusions demonstrate constitutive activation of the kinase, as measured by ROS1 phosphorylation (FIG. 1C). While each ROS1 fusion that we tested activated the JAK/STAT signaling pathway (measured by STAT3 phosphorylation) to an equivalent degree, the ability of ROS1 fusions to activate the RAS/MAPK pathway (measured by ERK phosphorylation) varied significantly across the different ROS1 fusion proteins tested (FIG. 1C). Both SDC4-ROS1 and SLC34A2-ROS1 fusions activated the MAPK pathway. In contrast, CD74-ROS1 fusions failed to as substantially induce RAS/MAPK pathway signaling (FIG. 1C). To confirm whether this differential activation of the MAPK pathway by different ROS1 fusions was recapitulated in patient-derived NSCLC models, we conducted short-term siRNA-mediated knockdown of ROS1 in ROS1 fusion-positive patient-derived NSCLC cell lines that express the same fusions studied in our isogenic system. We observed that knockdown of SDC4-ROS1 and SLC34A2-ROS1, but not CD74-ROS1, fusion proteins, resulted in suppression of the MAPK pathway (FIG. 1D-1G), thus corroborating our observations in the isogenic system.

### Example 3

### RAS/MAPK Pathway Signaling is Necessary and Sufficient for Survival of Cells Expressing ROS1 Fusion Oncoproteins that Specifically Activate RAS/MAPK Signaling

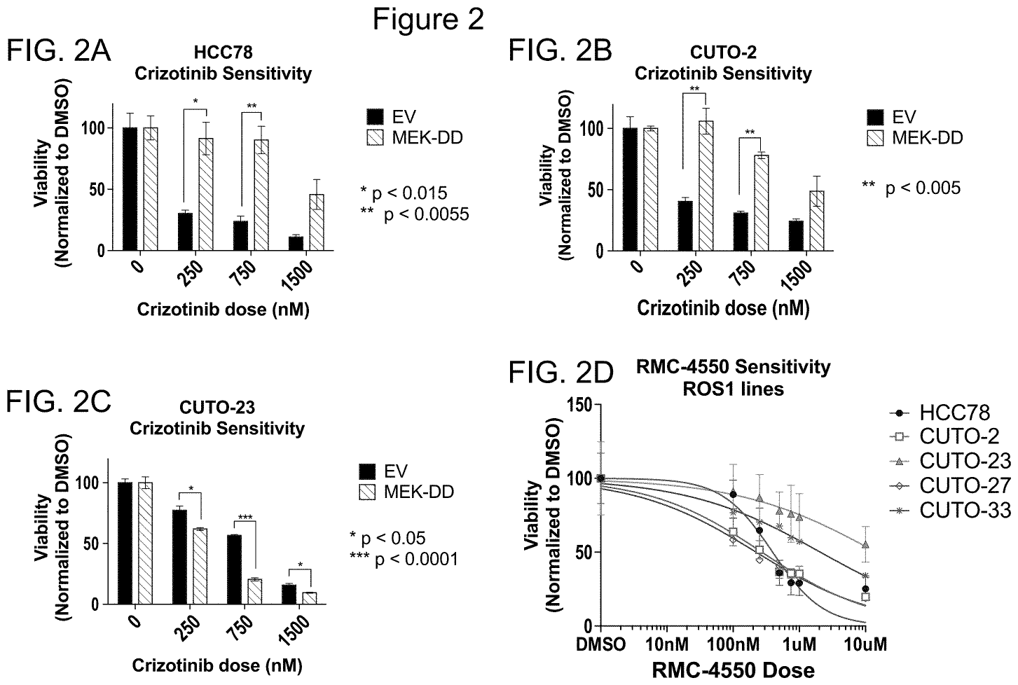

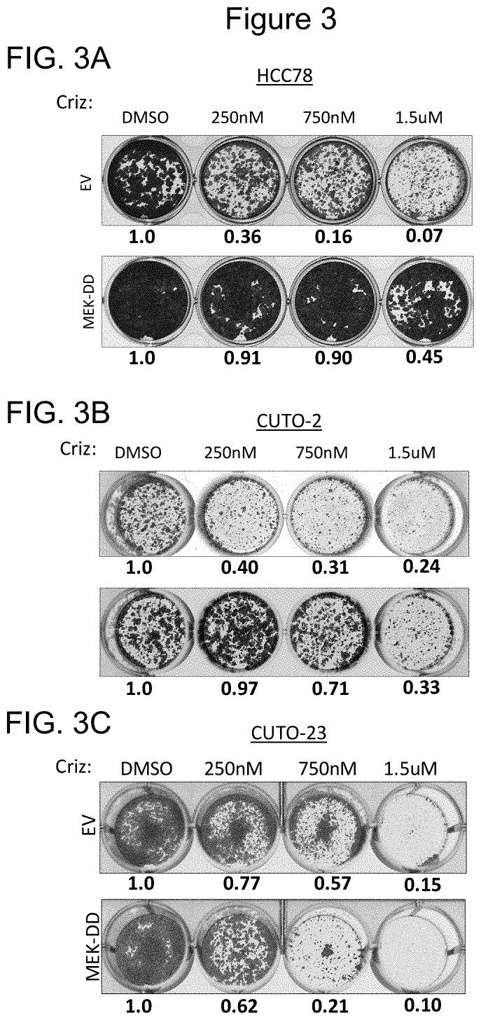

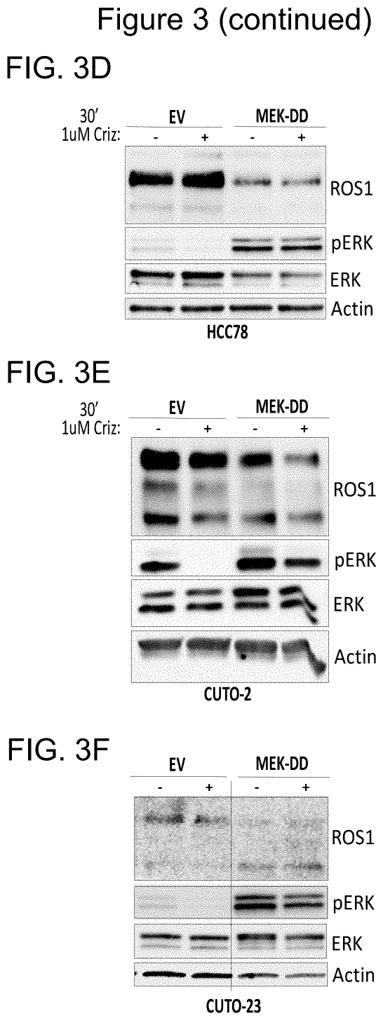

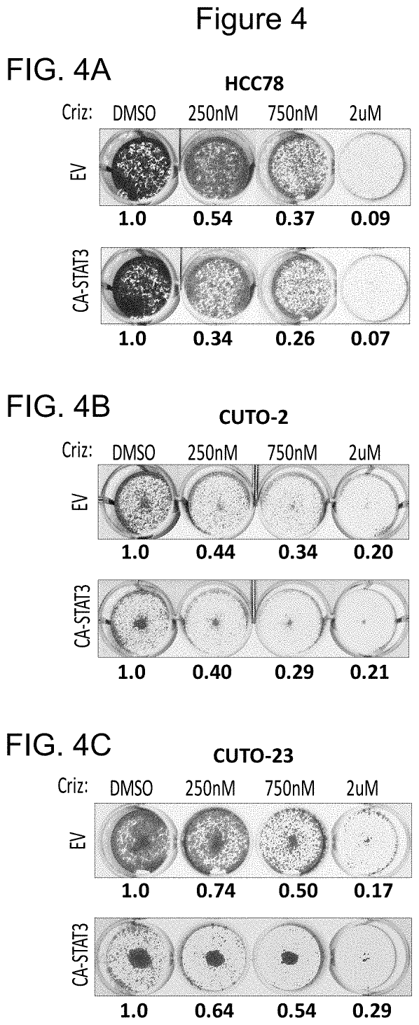

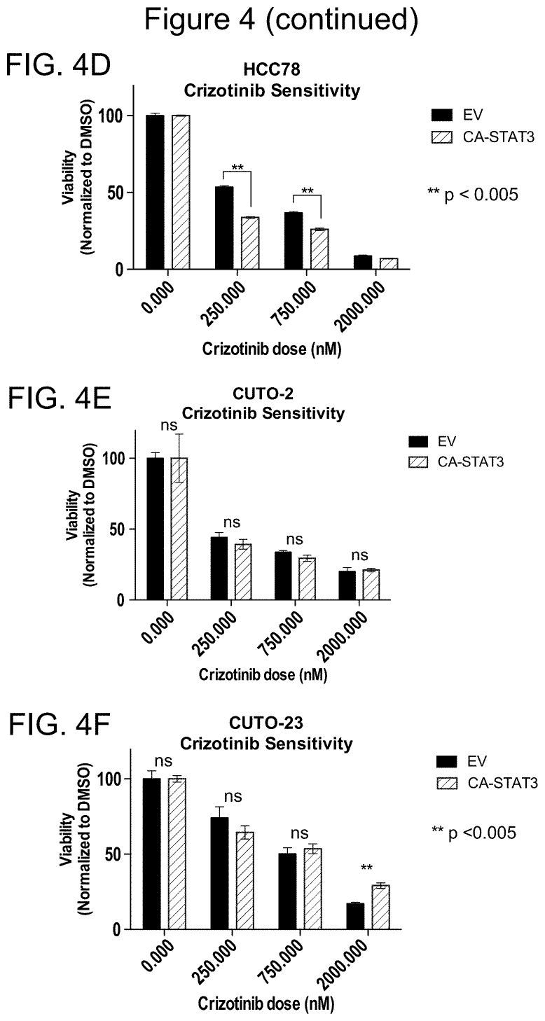

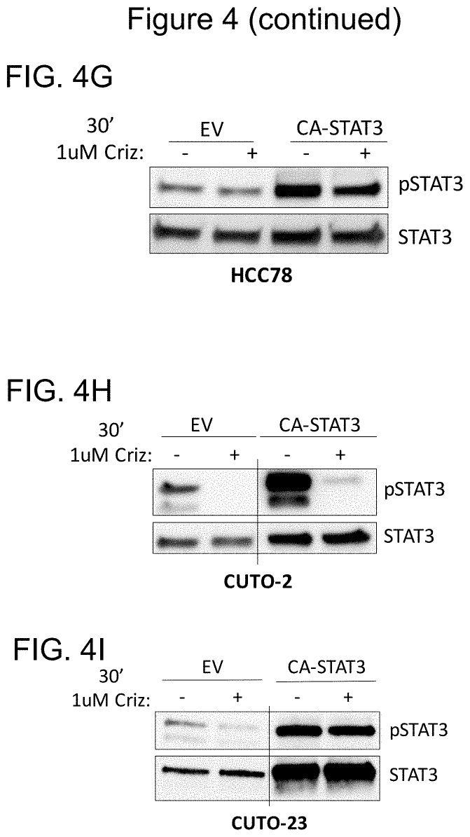

Based on these findings, we hypothesized that the MAPK pathway may play a more prominent role in controlling cell survival downstream of ROS1 fusion oncoproteins that we found can better engage this pathway, compared to those that are less capable. Indeed, we found that hyper-activation of the MAPK pathway by expression of a constitutively-active mutant form of MEK (MEK-DD) was sufficient to rescue cells expressing SDC4-ROS1 and SLC34A2-ROS1 fusions (which activate MAPK), but not a CD74-ROS1 fusion (which does not activate MAPK), from ROS1 inhibitor (crizotinib) sensitivity (FIG. 2A-C, FIG. 3) (Hrustanovic et al., 2015). In contrast, hyper-activation of JAK/STAT signaling by expression of a constitutively-active mutant form of STAT3 (CA-STAT3) was unable to rescue cells from crizotinib sensitivity across all ROS1 fusion oncoproteins tested, suggesting a less prominent role for JAK/STAT signaling in regulating cell survival in these systems (FIG. 4). (Hrustanovic et al., 2015)

### Example 4

### SHP2 Inhibition Effectively Kills Cancer Cells Harboring MAPK-Dependent ROS-1 Fusions

An emerging mechanism linking oncogenic RTK activation to downstream RAS/MAPK signaling involves the non-receptor protein tyrosine phosphatase SHP2, which is encoded by the PTPN11 gene and is critical for augmenting RAS-GTP levels and RAF-MEK-ERK activation. (Chen et al., 2016) SHP2 can also activate the JAK-STAT and/or the phosphoinositol 3-kinase-AKT pathways. SHP2 contributes to multiple cellular functions including proliferation, differentiation, cell cycle maintenance and migration.

Accordingly, we hypothesized that SHP2 promotes MAPK pathway activation downstream of the NSCLC ROS1 fusion oncoproteins. Indeed, SHP2 inhibition with an allosteric SHP2 inhibitor, RMC-4550, was effective in patient-derived NSCLC cell lines in which the MAPK pathway operated downstream of the ROS1 fusion (HCC78, CUTO-2), but not in cells in which the MAPK pathway was disconnected from the ROS1 fusion (CUTO-23, CUTO-33) (FIG. 2D-2E, FIG. 5). Our collective data show that in cells harboring SDC4-ROS1 and SLC34A2-ROS1 fusions, but not in those with the CD74-ROS1 fusion, MAPK pathway activation is necessary and sufficient for cell survival.

### Example 5

### Differential Subcellular Localization of ROS1 Oncoprotein Fusions Regulates Differential Signaling Pathway Activation

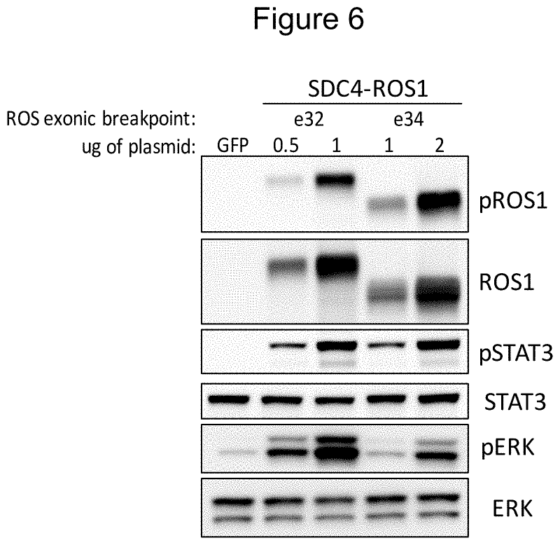

We examined several possible mechanisms that could underlie the differential signaling pathway activation operating downstream of the different ROS1 fusion oncoproteins. One possible mechanism is that the different exonic breakpoints that are present in the different fusion genes (e.g. ROS1 exon 32 versus exon 34 fused to an N-terminal partner) could contribute to differential pathway engagement. However, we found that the differential pathway activation observed downstream of a particular ROS1 fusion was similar whether the exonic breakpoint was in ROS1 exon 32 or 34 (FIG. 6). Furthermore, we noted that the entire ROS1 kinase domain was retained and identical between the different fusion forms based on DNA sequence analysis. We further found no significant differences in protein expression levels across the different fusion oncoproteins that could readily explain differential pathway engagement (FIG. 1C). Together, these data suggested a potential role for the N-terminal fusion partner in driving the differential signal pathway activation.

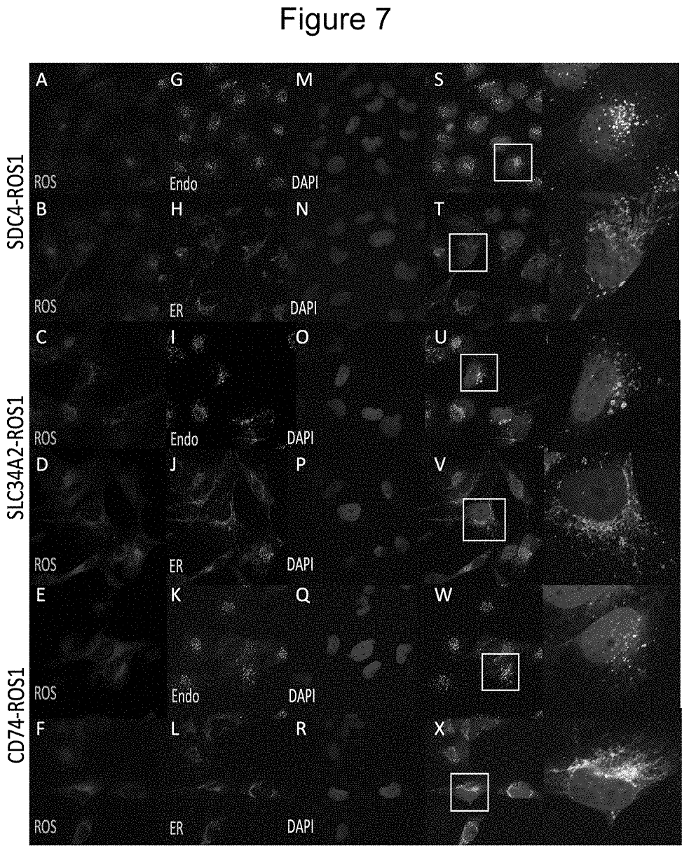

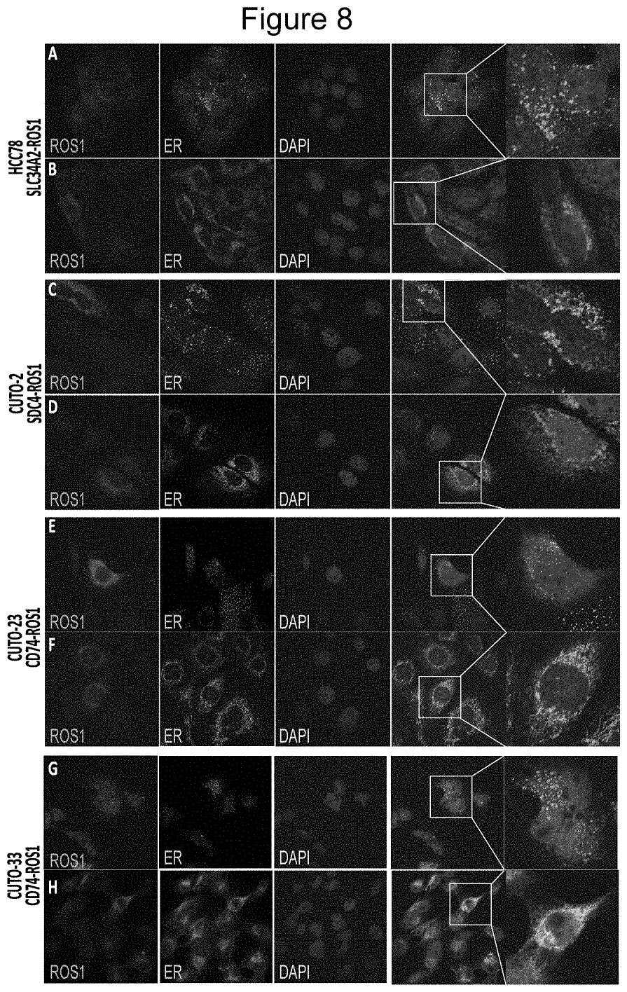

Using immunofluorescence and confocal microscopy analysis, we examined the subcellular localization of SDC4-ROS1, SLC34A2-ROS1, and CD74-ROS1 fusion proteins, both in isogenic BEAS2-B normal bronchial epithelial cell lines that we engineered to express these fusions (ROS1 B2Bs) and in the available ROS1 fusion patient-derived cell lines (FIG. 7, FIG. 8). We found that there was distinct subcellular distribution of the different ROS fusions. SDC4-ROS1 and SLC34A2-ROS1, which activate the MAPK pathway, were found in punctate structures that co-localized with the established endosomal marker EEA-1 (Mu et al., 1995). In contrast, CD74-ROS1, which does not substantially activate RAS/MAPK signaling, was localized in a different pattern that displayed perinuclear enhancement and co-localized with calnexin and PTP1B, established markers of the ER. (Ahluwalia et al., 1992). These data indicated that differential subcellular compartment localization correlated with differential MAPK pathway activation downstream of the different ROS1 oncoprotein fusions containing distinct N-terminal fusion partners.

### Example 6

### Re-Localization of CD74-ROS1 to Endosomes Induces RAS/MAPK Pathway Activation

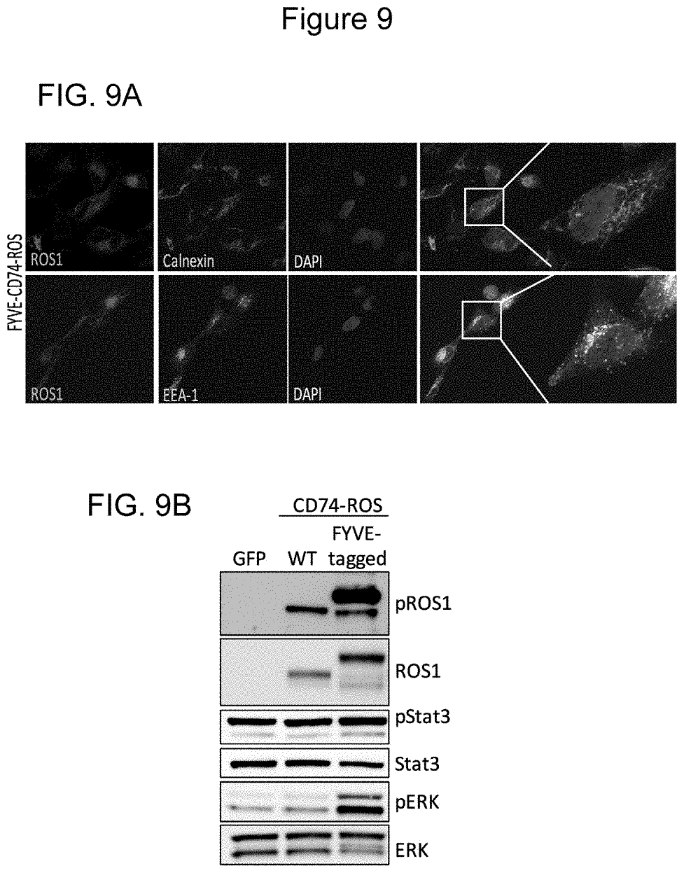

We next directly tested whether subcellular localization was required for pathway activation. Wildtype CD74 encodes the invariant chain, a type II transmembrane receptor which is involved in trafficking of MHC molecules through the ER to the endo-lysosome. CD74 contains a 15 amino acid N-terminal cytoplasmic extension, which anchors it into the ER. (Khalil et al., 2005; Schröder, 2016) We created a FYVE zinc finger domain-tagged CD74-ROS construct to re-localize the fusion protein to endosomes. (Hayakawa et al., 2004) Immunofluorescence analysis of ROS1 in BEAS2-B cells expressing this construct showed re-localization of the FYVE-CD74-ROS1 protein from the ER to punctate structures where it partly co-localized with the endosomal marker EEA-1, reminiscent of SDC4-ROS1 and SLC34A2-ROS1 subcellular localization (FIG. 9A). Furthermore, in contrast to CD74-ROS1, expression of the FYVE-CD74-ROS1 protein induced MAPK pathway activation, suggesting that the specific subcellular localization of ROS1 fusion oncoproteins is critical in mediating RAS/MAPK pathway signaling (FIG. 9B). No difference in STAT3 phosphorylation was observed, suggesting pathway specificity in the signaling phenotype that is regulated via differential subcellular compartment regulation (FIG. 9B). Thus, the differential MAPK pathway activation that is observed between different ROS1 fusions is controlled by fusion-specific and distinct subcellular compartment localization, which is conferred by the N-terminal fusion partner.

### Example 7

### RAS/MAPK Pathway-Activating ROS1 Fusions Form More Aggressive Tumors In Vivo

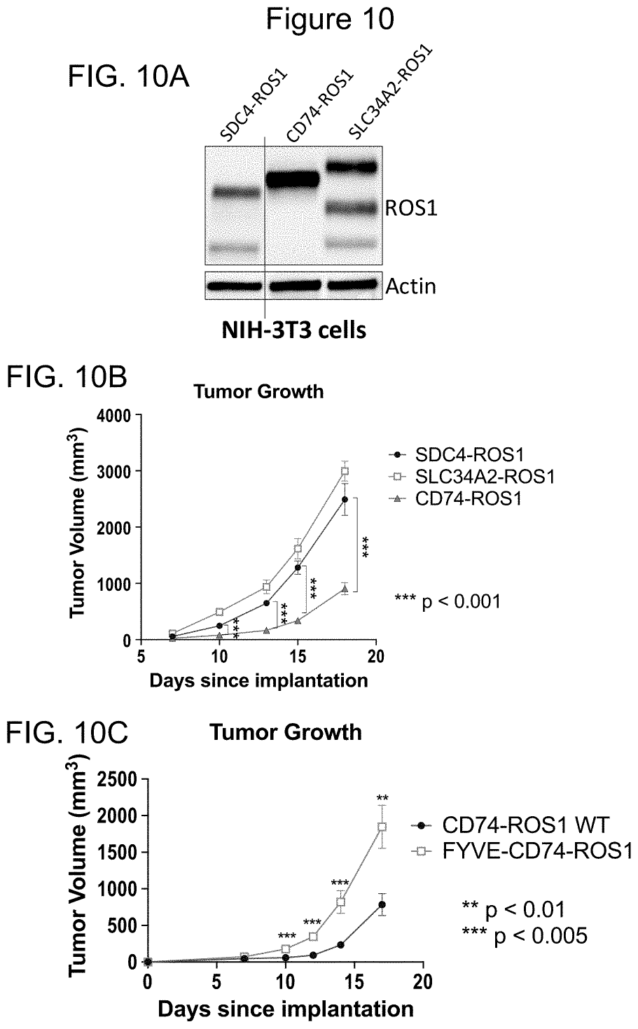

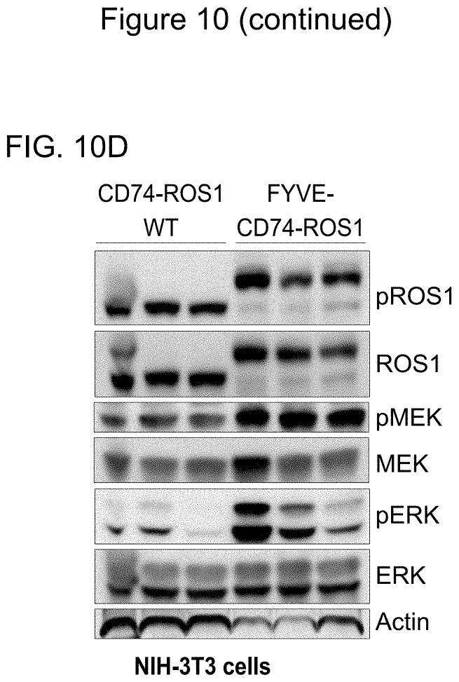

We next investigated the potential oncogenic significance of the differential ability of these ROS1 fusions to activate the RAS/MAPK pathway. Despite multiple attempts, none of the limited number of ROS1 fusion-positive patient-derived lines that are currently available grew successfully as tumor xenografts in immunocompromised mice and patient-derived xenograft (PDX) models are not currently available. Thus, to examine tumor growth in vivo, we generated a genetically-controlled isogenic system in which NIH-3T3 cells were engineered to express the SDC4-ROS1 and SLC34A2-ROS1 (that activate MAPK signaling) and CD74-ROS1 (that does not activate MAPK signaling) fusions (FIG. 10A). Standard tumor xenograft studies in immunocompromised mice were conducted to assess for differential oncogenic properties in vivo. As expected, NIH-3T3 cells expressing all three ROS1 fusions formed tumors in mice, while control NIH-3T3 cells expressing an empty vector did not (FIG. 10B and data not shown). Interestingly, the SDC4-ROS1 and SLC34A2-ROS1 fusion expressing cells formed more aggressive tumors, as assessed by growth rate in vivo, compared to CD74-ROS1 fusion-driven tumors (FIG. 10B). Additionally, we generated NIH-3T3 cells expressing the endosomal-targeted FYVE-CD74-ROS1 fusion protein, which is able to activate the MAPK pathway, and compared the in vivo growth rate of those cells to NIH-3T3 cells expressing wildtype CD74-ROS1, which do not demonstrate substantial MAPK pathway activation (FIG. 10C). Intriguingly, we found that the FYVE-CD74-ROS1 tumors grow at a significantly faster rate than wildtype CD74-ROS1 (FIG. 10D). These data suggest that expression of a ROS1 fusion oncoprotein that can activate the MAPK pathway as a consequence of re-localization to endosomes results in tumors that are more aggressive compared to tumors expressing ROS1 fusion oncoproteins that do not activate MAPK and localize to the ER.

### Conclusion

Our findings provide evidence demonstrating for the first time that the specific N-terminal fusion partners present within gene rearrangements involving the same RTK partner can directly control differential signaling pathway engagement by causing alternative subcellular compartment localization. These findings have implications for the understanding of the molecular and cell biological basis of cancer growth and establish a link between differential subcellular localization of oncoprotein RTKs with their oncogenic mechanism and properties. Such studies provide fundamental insight and could be critical for the design of novel diagnostic and therapeutic strategies to improve clinical outcomes.

Genomic advances have led to the more precise genetic classification of tumors including NSCLCs, with improvements in clinical outcomes through genotype-directed targeted therapy. One prominent example is the 19-month progression-free survival observed in patients harboring ROS1 fusion-positive NSCLC treated with ROS1 inhibitors such as crizotinib. (Shaw and Solomon, 2015) In current clinical practice worldwide, the diagnosis of ROS1 fusions in cancers most commonly occurs via a break-apart FISH (Fluorescence In Situ Hybridization) assay. Consequently, the specific N-terminal fusion partner is not identified. Our study demonstrates that while all ROS fusions examined activate the JAK/STAT pathway to a similar degree, they vary in their ability to activate the MAPK pathway. SDC4-ROS1 and SFC34A2-ROS1 fusions activate the MAPK pathway while CD74-ROS1 does not. We found that for ROS1, this differential MAPK pathway activation is due to differential subcellular compartment localization of the different ROS1 fusion.

The patient-derived CD74-ROS1 cDNA utilized in our studies contains an ER-targeting motif, which anchors the ROS1 fusion to the ER, and limits its ability to activate MAPK. Interestingly, the shorter isoform of wildtype CD74 lacks this N-terminal ER-targeting motif, leaving open the possibility that some CD74-ROS1 tumors may express this shorter isoform and may be able to engage MAPK. (Schröder, 2016) In our studies, the ability of individual fusion proteins to activate the MAPK pathway is correlated with tumor aggressiveness, suggesting that current diagnostics identifying only the presence or absence of a fusion oncoprotein in a binary manner may be insufficient. More precise identification of the fusion partner (e.g. via next-generation DNA or RNA sequencing) may be critical to better stratify patients for treatment (either single-agent or combination therapy). While many ROS1 fusion-positive tumors initially respond to crizotinib, virtually all tumors become resistant to therapy. We found that MAPK pathway activation is necessary and sufficient for survival of cells expressing SDC4-ROS1 and SLC34A2-ROS1, suggesting MAPK pathway reactivation may be a mechanism of resistance to crizotinib monotherapy. Consistent with this notion, there are limited reports of RAS activating mutations or upregulation driving resistance to crizotinib in the setting of these ROS1 fusion driven cancers. (Cargnelutti et al., 2015; Zhu et al., 2017)

### Example 8

### Effect of SHP2 Inhibition on ERK Phosphorylation and Proliferation of Additional Oncogenic Tyrosine Kinase Fusion Cell Lines

In order to extend the above results to other tyrosine kinase fusions, we tested whether SHP2 inhibition with RMC-4550 was able to inhibit ERK phosphorylation and proliferation in vitro in NCI-H3122 and LC-2/AD lung adenocarcinoma cells, which contain EML4-ALK and CCDC6-RET fusions, respectively, and which have been shown previously to activate MAPK signaling. The experiments were performed as described below in the Example 8 Methods section.

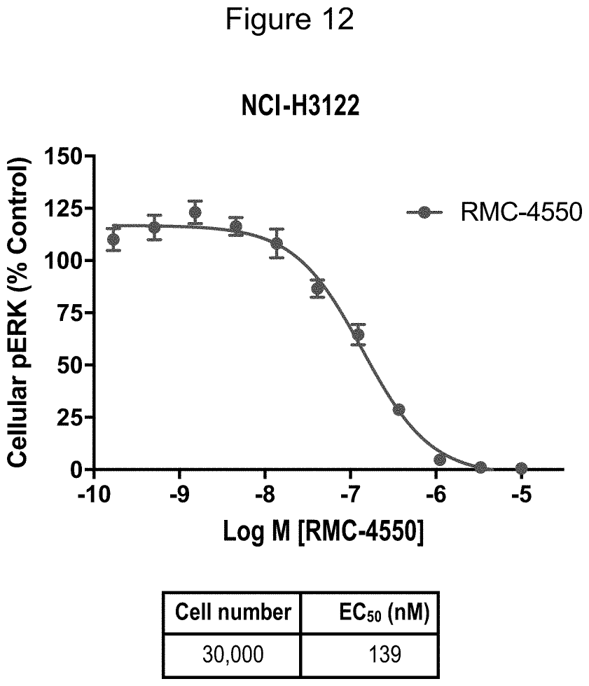

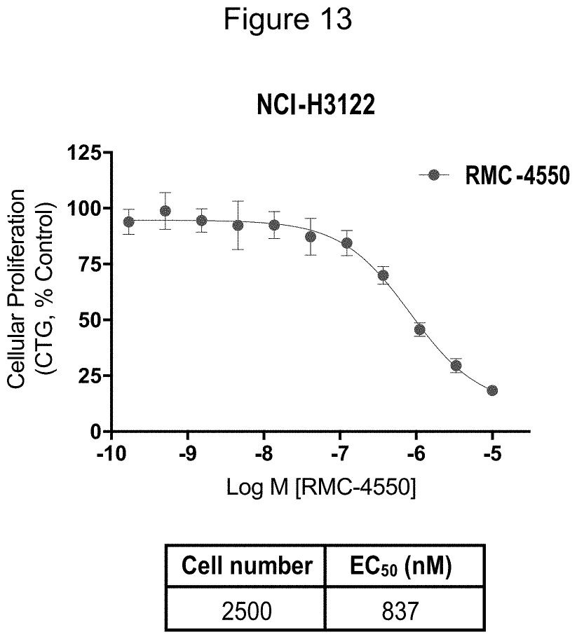

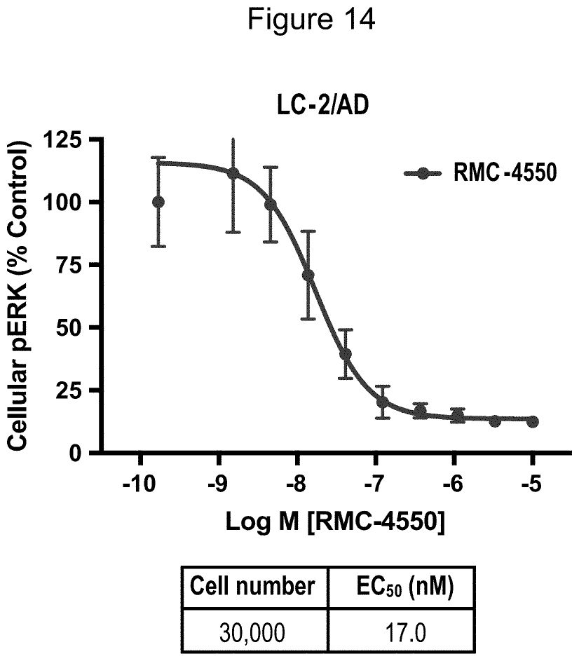

As shown in FIG. 12 and FIG. 13, treatment of NCI-H3122 cells with RMC-4550 produce dose-dependent inhibition of ERK phosphorylation (EC50 of 139 nM) and cellular proliferation (EC50 of 837 nM), respectively. Similarly, as shown in FIG. 14, RMC-4550 treatment of LC-2/AD cells resulted in dose-dependent inhibition of ERK phosphorylation (EC50 of 17 nM), These results confirm the above results that tyrosine-kinase fusions that activate MAPK signaling are susceptible to SHP2-inhibition.

Thus, the data presented herein supports the implementation of a precision diagnostic step to the treatment of cancers driven by tyrosine kinase fusions, whereby patients having tyrosine kinase fusions that activate the MAPK pathway should be stratified into a treatment group that receives a SHP2 inhibitor (alone or in combination with one or more additional therapeutic agents, e.g., a MEK inhibitors) and whereby patients that have tyrosine kinase fusions that do not activate the MAPK pathway should be treated with alternative therapies.

### Example 8 Methods

EML4-ALK Fusion Line—Phospho-ERK (pERK) (FIG. 12 Data)

NCI-H3122 cells were seeded in 96-well format plates at a density of 30000 cells/well in complete media, and incubated at 37° C. in 5% CO2 overnight. Approximately 18 hours after seeding, cells were treated with RMC-4550 at concentrations ranging from 10 uM to ˜170 pM or 0.1% DMSO as a vehicle control for 60 minutes at 37° C. in 5% CO2. Cellular lysates were prepared and pERK levels assayed using the AlphaLISA® SureFire® Ultra HV pERK Assay Kit (Perkin Elmer).

EML4-ALK Fusion Line—Cell Proliferation (FIG. 13 Data)

NCI-H3122 cells were seeded in 96-well format ultra-low adhesion plates at a density of 2500 or 5000 cells/well, centrifuged at 300×g for 10 minutes, and incubated in complete media at 37° C. in 5% CO₂ for 72 hours to induce spheroid formation. Cells were treated with RMC-4550 at concentrations ranging from 10 uM to ˜170 pM or 0.1% DMSO as a vehicle control for 5 days at 37° C. in 5% CO₂. Cell viability was assessed using the 3D CellTiter-Glo® (CTG) kit (Promega).

CCDC6-RET Fusion Line—Phospho-ERK (pERK) (FIG. 14 Data)

LC-2/AD lung adenocarcinoma cells were seeded in 96-well format plates at a density of 20000, 30000, or 40000 cells/well in complete media, and incubated at 37° C. in 5% CO2 overnight. Approximately 18 hours after seeding, cells were treated with RMC-4550 at concentrations ranging from 10 uM to ˜170 pM or 0.1% DMSO as a vehicle control for 60 minutes at 37° C. in 5% CO2. Cellular lysates were prepared and pERK levels assayed using the AlphaLISA® SureFire® Ultra HV pERK Assay Kit (Perkin Elmer).

### Example 9

### SHP2 Allosteric Inhibition Assay

Objective: To demonstrate the inhibition of SHP2 activity with RMC-3943, RMC-4550, and Compound C.

Without wishing to be bound by theory, SHP is allosterically activated through binding of bis-tyrosyl-phosphorylated peptides to its Src Homology 2 (SH2) domains. The latter activation step leads to the release of the auto-inhibitory interface of SHP2, which in turn renders the SHP2 protein tyrosine phosphatase (PTP) active and available for substrate recognition and reaction catalysis. The catalytic activity of SHP2 was monitored using the surrogate substrate DiFMUP in a prompt fluorescence assay format.

The phosphatase reactions were performed at room temperature in 96-well black polystyrene plate, flat bottom, non-binding surface (Corning, Cat #3650) using a final reaction volume of 100 μL and the following assay buffer conditions: 50 mM HEPES, pH 7.2, 100 mM NaCl, 0.5 mM EDTA, 0.05% P-20, 1 mM DTT.

The inhibition of SHP2 by RMC-3943, RMC-4550, and Compound C was monitored using an assay in which 0.2 nM of SHP2 was incubated with 0.5 μM of Activating Peptide 1 (sequence: H₂N-LN(pY)IDLDLV(dPEG8)LST(pY)ASINFQK-amide) (SEQ ID NO: 1) or Activating Peptide 2 (sequence: H₂N-LN(pY)AQLWHA(dPEG8)LTI(pY)ATIRRF-amide) (SEQ ID NO: 2). After 30-60 minutes incubation at 25° C., the surrogate substrate DiFMUP (Invitrogen, Cat #D6567) was added to the reaction and activity was determined by a kinetic read using a microplate reader (Envision, Perkin-Elmer or Spectramax M5, Molecular Devices). The excitation and emission wavelengths were 340 nm and 450 nm, respectively. Initial rates were determined from a linear fit of the data, and the inhibitor dose response curves were analyzed using normalized IC₅₀ regression curve fitting with control based normalization.

Using the above-protocol, SHP2 inhibition by RMC-3943, RMC-4550, and Compound C is shown in Table 2.

TABLE 2
SHP2 Inhibition by RMC-3943, RMC-4550, and Compound C

| Compound | SHP2 IC₅₀, nM |
|---|---|
| RMC-3943 | 2.19 |
| RMC-4550 | 1.55 |
| Compound C | 1.29 |

### REFERENCES

Acquaviva, J., Wong, R., Charest, A., 2009. The multifaceted roles of the receptor tyrosine kinase ROS in development and cancer. Biochim. Biophys. Acta 1795, 37-52. doi: 10.1016/j.bbcan.2008.07.006
Ahluwalia, N., Bergeron, J. J., Wada, I., Degen, E., Williams, D. B., 1992. The p88 molecular chaperone is identical to the endoplasmic reticulum membrane protein, calnexin. J. Biol. Chem. 267, 10914-10918.
Bergethon, K., Shaw, A. T., Ou, S.-H. I., Katayama, R., Lovly, C. M., McDonald, N. T., Massion, P. P., Siwak-Tapp, C., Gonzalez, A., Fang, R., Mark, E. J., Batten, J. M., Chen, H., Wilner, K. D., Kwak, E. L., Clark, J. W., Carbone, D. P., Ji, E L, Engelman, J. A., Mino-Kenudson, M., Pao, W., Iafrate, A. J., 2012. ROS1 rearrangements define a unique molecular class of lung cancers. J. Clin. Oncol. 30, 863-870. doi:10.1200/JC0.2011.35.6345
Cargnelutti, M., Corso, S., Pergolizzi, M., Mévellec, L., Aisner, D. L., Dziadziuszko, R., Varella-Garcia, M., Comoglio, P. M., Doebele, R. C., Vialard, J., Giordano, S., 2015. Activation of RAS family members confers resistance to ROS1 targeting drugs. Oncotarget 6, 5182-5194. doi:10.18632/oncotarget.3311
Charest, Al, Wilker, E. W., McLaughlin, M. E., Lane, K., Gowda, R., Coven, S., McMahon, K., Kovach, S., Feng, Y., Yaffe, M. B., Jacks, T., Housman, D., 2006. ROS fusion tyrosine kinase activates a SH2 domain-containing phosphatase-2/phosphatidylinositol 3-kinase/mammalian target of rapamycin signaling axis to form glioblastoma in mice. Cancer Res. 66, 7473-7481. doi:10.1158/0008-5472.CAN-06-1193
Charest, Alan, Kheifets, V., Park, J., Lane, K., McMahon, K., Nutt, C. L., Housman, D., 2003. Oncogenic targeting of an activated tyrosine kinase to the Golgi apparatus in a glioblastoma. Proc. Natl. Acad. Sci. U.S.A. 100, 916-921. doi:10.1073/pnas.242741799
Chen, Y.-N. P., LaMarche, M. J., Chan, H. M., Fekkes, P., Garcia-Fortanet, J., Acker, M. G., Antonakos, B., Chen, C. H.-T., Chen, Z., Cooke, V. G., Dobson, J. R., Deng, Z., Fei, F., Firestone, B., Fodor, M., Fridrich, C., Gao, H., Grunenfelder, D., Hao, H.-X., Jacob, J., Ho, S., Hsiao, K., Kang, Z. B., Karki, R., Kato, M., Larrow, J., La Bonte, L. R., Lenoir, F., Liu, G., Liu, S., Majumdar, D., Meyer, M. J., Palermo, M., Perez, L., Pu, M., Price, E., Quinn, C., Shakya, S., Shultz, M. D., Slisz, J., Venkatesan, K., Wang, P., Warmuth, M., Williams, S., Yang, G., Yuan, J., Zhang, J.-H., Zhu, P., Ramsey, T., Keen, N. J., Sellers, W. R., Stams, T., Fortin, P. D., 2016. Allosteric inhibition of SFLP2 phosphatase inhibits cancers driven by receptor tyrosine kinases. Nature 535, 148-152. doi: 10.1038/nature 18621
Davies, K. D., Doebele, R. C., 2013. Molecular pathways: ROS1 fusion proteins in cancer. Clin. Cancer Res. 19, 4040-4045. doi:10.1158/1078-0432.CCR-12-2851
Dobson, L., Reményi, I., Tusnády, G. E., 2015a. CCTOP: a Consensus Constrained TOPology prediction web server. Nucleic Acids Res. 43, W408-12. doi:10.1093/nar/gkv451
Dobson, L., Reményi, I., Tusnády, G. E., 2015b. The human transmembrane proteome. Biol. Direct 10, 31. doi:10.1186/s13062-015-0061-x
Doebele, R. C., Pilling, A. B., Aisner, D. L., Kutateladze, T. G., Le, A. T., Weickhardt, A. J., Kondo, K. L., Linderman, D. J., Heasley, L. E., Franklin, W. A., Varella-Garcia, M., Camidge, D. R., 2012. Mechanisms of resistance to crizotinib in patients with ALK gene rearranged non-small cell lung cancer. Clin. Cancer Res. 18, 1472-1482. doi: 10.1158/1078-0432.CCR-11-2906
Engelman, J. A., Zejnullahu, K., Mitsudomi, T., Song, Y., Hyland, C., Park, J. O., Lindeman, N., Gale, C.-M., Zhao, X., Christensen, J., Kosaka, T., Holmes, A. J., Rogers, A. M., Cappuzzo, F., Mok, T., Lee, C., Johnson, B. E., Cantley, L. C., Jänne, P. A., 2007. MET amplification leads to gefitinib resistance in lung cancer by activating ERBB3 signaling. Science 316, 1039-1043. doi: 10.1126/science. 1141478
Forbes, S. A., Beare, D., Boutselakis, H., Bamford, S., Bindal, N., Tate, J., Cole, C. G., Ward, S., Dawson, E., Ponting, L., Stefancsik, R., Harsha, B., Kok, C. Y., Jia, M., Jubb, H., Sondka, Z., Thompson, S., De, T., Campbell, P. J., 2017. COSMIC: somatic cancer genetics at high-resolution. Nucleic Acids Res. 45, D777-D783. doi:10.1093/nar/gkw1121
Govindan, R., Ding, L., Griffith, M., Subramanian, J., Dees, N. D., Kanchi, K. L., Maher, C. A., Fulton, R., Fulton, L., Wallis, J., Chen, K., Walker, J., McDonald, S., Bose, R., Ornitz, D., Xiong, D., You, M., Dooling, D. J., Watson, M., Mardis, E. R., Wilson, R. K., 2012. Genomic landscape of non-small cell lung cancer in smokers and never-smokers. Cell 150, 1121-1134. doi:10.1016/j.cell.2012.08.024
Hayakawa, A., Hayes, S. J., Lawe, D. C., Sudharshan, E., Tuft, R., Fogarty, K., Lambright, D., Corvera, S., 2004. Structural basis for endosomal targeting by FYVE domains. J. Biol. Chem. 279, 5958-5966. doi: 10.1074/jbc.M310503200
Hrustanovic, G., Olivas, V., Pazarentzos, E., Tulpule, A., Asthana, S., Blakely, C. M., Okimoto, R. A., Lin, L., Neel, D. S., Sabnis, A., Flanagan, J., Chan, E., Varella-Garcia, M., Aisner, D. L., Vaishnavi, A., Ou, S.-H. I., Collisson, E. A., Ichihara, E., Mack, P. C., Lovly, C. M., Karachaliou, N., Rosell, R., Riess, J. W., Doebele, R. C., Bivona, T. G., 2015. RAS-MAPK dependence underlies a rational polytherapy strategy in EML4-ALK-positive lung cancer. Nat. Med. 21, 1038-1047. doi:10.1038/nm.3930
Khalil, H., Brunet, A., Thibodeau, J., 2005. A three-amino-acid-long HLA-DRbeta cytoplasmic tail is sufficient to overcome ER retention of invariant-chain p35. J. Cell. Sci. 118, 4679-4687. doi:10.1242/jcs.02592
Kobayashi, S., Boggon, T. J., Dayaram, T., Jänne, P. A., Kocher, O., Meyerson, M., Johnson, B. E., Eck, M. J., Tenen, D. G., Halmos, B., 2005. EGFR mutation and resistance of non-small-cell lung cancer to gefitinib. N. Engl. J. Med. 352, 786-792. doi: 10.1056/NE JMoa044238
Kohno, T., Nakaoku, T., Tsuta, K., Tsuchihara, K., Matsumoto, S., Yoh, K., Goto, K., 2015. Beyond ALK-RET, ROS1 and other oncogene fusions in lung cancer. Transl Lung Cancer Res 4, 156-164. doi: 10.3978/j issn.2218-6751.2014.11.11
Li, C., Sun, Y., Fang, R., Han, X., Luo, X., Wang, R., Pan, Y., Hu, H., Zhang, Y., Pao, W., Shen, L., Ji, H., Chen, H., 2012. Lung adenocarcinomas with HER2-activating mutations are associated with distinct clinical features and HER2/EGFR copy number gains. J Thorac Oncol 7, 85-89. doi:10.1097/JTO.0b013e318234f0a2
Lynch, T. J., Bell, D. W., Sordella, R., Gurubhagavatula, S., Okimoto, R. A., Brannigan, B. W., Harris, P. L., Haserlat, S. M., Supko, J. G., Haluska, F. G., Louis, D. N., Christiani, D. C., Settleman, J., Haber, D. A., 2004. Activating mutations in the epidermal growth factor receptor underlying responsiveness of non-small-cell lung cancer to gefitinib. N. Engl. J. Med. 350, 2129-2139. doi:10.1056/NEJMoa040938
Medves, S., Demoulin, J.-B., 2012. Tyrosine kinase gene fusions in cancer: translating mechanisms into targeted therapies. J. Cell. Mol. Med. 16, 237-248. doi: 10.1111/j. 1582-4934.2011.01415.x
Mu, F. T., Callaghan, J. M., Steele-Mortimer, O., Stenmark, H., Parton, R. G., Campbell, P. L., McCluskey, J., Yeo, J. P., Tock, E. P., Toh, B. H., 1995. EEA1, an early endosome-associated protein. EEA1 is a conserved alpha-helical peripheral membrane protein flanked by cysteine “fingers” and contains a calmodulin-binding IQ motif. J. Biol. Chem. 270, 13503-13511.
Nichols, R. J., Haderk, F., Stahlhut, C., Schulze, C. J., Hemmati, G., Wildes, D., Tzitzilonis, C., Mordec, K., Marquez, A., Romero, J., Hsieh, D., Kiss, G., Koltun, E. S., Gill, A. L., Singh, M., Goldsmith, M. A., Smith, J. A. M., Bivona, T. G., 2017. Efficacy of SHP2 phosphatase inhibition in cancers with nucleotide-cycling oncogenic RAS, RAS-GTP dependent oncogenic BRAF and NF1 loss, doi: 10.1101/188730
Ou, S.-H. I., Ahn, J. S., De Petris, L., Govindan, R., Yang, J. C.-H., Hughes, B., Léna, H., Moro-Sibilot, D., Bearz, A., Ramirez, S. V., Mekhail, T., Spira, A., Bordogna, W., Balas, B., Morcos, P. N., Monnet, A., Zeaiter, A., Kim, D.-W., 2016. Alectinib in Crizotinib-Refractory ALK-Rearranged Non-Small-Cell Lung Cancer: A Phase II Global Study. J. Clin. Oncol. 34, 661-668. doi: 10.1200/jco.2015.63.9443
Paez, J. G., Jänne, P. A., Lee, J. C., Tracy, S., Greulich, H., Gabriel, S., Herman, P., Kaye, F. J., Lindeman, N., Boggon, T. J., Naoki, K., Sasaki, H., Fujii, Y., Eck, M. J., Sellers, W. R., Johnson, B. E., Meyerson, M., 2004. EGFR mutations in lung cancer: correlation with clinical response to gefitinib therapy. Science 304, 1497-1500. doi: 10.1126/science. 1099314
Pao, W., Hutchinson, K. E., 2012. Chipping away at the lung cancer genome. Nat. Med. 18, 349-351. doi: 10.1038/nm.2697
Pao, W., Miller, V. A., Politi, K. A., Riely, G. J., Somwar, R., Zakowski, M. F., Kris, M. G., Varmus, H., 2005. Acquired resistance of lung adenocarcinomas to gefitinib or erlotinib is associated with a second mutation in the EGFR kinase domain. PLoS Med. 2, e73. doi: 10.1371/journal.pmed.0020073
Rikova, K., Guo, A., Zeng, Q., Possemato, A., Yu, J., Haack, H., Nardone, J., Lee, K., Reeves, C., Li, Y., Hu, Y., Tan, Z., Stokes, M., Sullivan, L., Mitchell, J., Wetzel, R., Macneill, J., Ren, J. M., Yuan, J., Bakalarski, C. E., Villen, J., Kornhauser, J. M., Smith, B., Li, D., Zhou, X., Gygi, S. P., Gu, T.-L., Polakiewicz, R. D., Rush, J., Comb, M. J., 2007. Global survey of phosphotyrosine signaling identifies oncogenic kinases in lung cancer. Cell 131, 1190-1203. doi: 10.1016/j cell.2007.11.025
Rotow, J., Bivona, T. G., 2017. Understanding and targeting resistance mechanisms in NSCLC. Nat. Rev. Cancer 17, 637-658. doi:10.1038/nrc.2017.84
Schröder, B., 2016. The multifaceted roles of the invariant chain CD74-More than just a chaperone. Biochim. Biophys. Acta 1863, 1269-1281. doi:10.1016/j.bbamcr.2016.03.026
Seo, J.-S., Ju, Y. S., Lee, W.-C., Shin, J.-Y., Lee, J. K., Bleazard, T., Lee, J., Jung, Y. J., Kim, J.-O., Shin, J.-Y., Yu, S.-B., Kim, J., Lee, E.-R., Kang, C.-H., Park, I.-K., Rhee, H., Lee, S.-H., Kim, J.-I., Kang, J.-H., Kim, Y. T., 2012. The transcriptional landscape and mutational profile of lung adenocarcinoma. Genome Res. 22, 2109-2119. doi: 10.1101/gr. 145144.112
Shaw, A. T., Kim, D.-W., Mehra, R., Tan, D. S. W., Felip, E., Chow, L. Q. M., Camidge, D. R., Vansteenkiste, J., Sharma, S., De Pas, T., Riely, G. J., Solomon, B. J., Wolf, J., Thomas, M., Schuler, M., Liu, G., Santoro, A., Lau, Y. Y., Goldwasser, M., Boral, A. L., Engelman, J. A., 2014. Ceritinib in ALK-rearranged non-small-cell lung cancer. N. Engl. J. Med. 370, 1189-1197. doi:10.1056/NEJMoa1311107
Shaw, A. T., Solomon, B. J., 2015. Crizotinib in RO SI-rearranged non-small-cell lung cancer. N. Engl. J. Med. 372, 683-684. doi:10.1056/NEJMc1415359
Stephens, P., Hunter, C., Bignell, G., Edkins, S., Davies, H., Teague, J., Stevens, C., O'Meara, S., Smith, R., Parker, A., Barthorpe, A., Blow, M., Brackenbury, L., Butler, A., Clarke, O., Cole, J., Dicks, E., Dike, A., Drozd, A., Edwards, K., Forbes, S., Foster, R., Gray, K., Greenman, C., Halliday, K., Hills, K., Kosmidou, V., Lugg, R., Menzies, A., Perry, J., Petty, R., Raine, K., Ratford, L., Shepherd, R., Small, A., Stephens, Y., Tofts, C., Varian, J., West, S., Widaa, S., Yates, A., Brasseur, F., Cooper, C. S., Flanagan, A. M., Knowles, M., Leung, S. Y., Louis, D. N., Looijenga, L. H. J., Malkowicz, B., Pierotti, M. A., Teh, B., Chenevix-Trench, G., Weber, B. L., Yuen, S. T., Harris, G., Goldstraw, P., Nicholson, A. G., Futreal, P. A., Wooster, R., Stratton, M. R., 2004. Lung cancer: intragenic ERBB2 kinase mutations in tumours. Nature 431, 525-526. doi: 10.1038/431525b
Takeuchi, K., Choi, Y. L., Togashi, Y., Soda, M., Hatano, S., Inamura, K., Takada, S., Ueno, T., Yamashita, Y., Satoh, Y., Okumura, S., Nakagawa, K., Ishikawa, Y., Mano, H., 2009. KIF5B-ALK, a novel fusion oncokinase identified by an immunohistochemistry-based diagnostic system for ALK-positive lung cancer. Clin. Cancer Res. 15, 3143-3149. doi: 10.1158/1078-0432.CCR-08-3248
Takeuchi, K., Soda, M., Togashi, Y., Suzuki, R., Sakata, S., Hatano, S., Asaka, R., Hamanaka, W., Ninomiya, H., Uehara, H., Lim Choi, Y., Satoh, Y., Okumura, S., Nakagawa, K., Mano, H., Ishikawa, Y., 2012. RET, ROS1 and ALK fusions in lung cancer. Nat. Med. 18, 378-381. doi:10.1038/nm.2658
Vaishnavi, A., Capelletti, M., Le, A. T., Kako, S., Butaney, M., Ercan, D., Mahale, S., Davies, K. D., Aisner, D. L., Pilling, A. B., Berge, E. M., Kim, J., Sasaki, E L, Park, S., Kryukov, G., Garraway, L. A., Hammerman, P. S., Haas, J., Andrews, S. W., Lipson, D., Stephens, P. J., Miller, V. A., Varella-Garcia, M., Jänne, P. A., Doebele, R. C., 2013. Oncogenic and drug-sensitive NTRK1 rearrangements in lung cancer. Nat. Med. 19, 1469-1472. doi:10.1038/nm.3352
Yun, C.-H., Mengwasser, K. E., Toms, A. V., Woo, M. S., Greulich, H., Wong, K.-K., Meyerson, M., Eck, M. J., 2008. The T790M mutation in EGFR kinase causes drug resistance by increasing the affinity for ATP. Proc. Natl. Acad. Sci. U.S.A. 105, 2070-2075. doi: 10.1073/pnas.0709662105
Zhu, Y.-C., Lin, X.-P., Li, X.-F., Wu, L.-X., Chen, H.-F., Wang, W.-X., Xu, C.-W., Shen, I-F., Wei, J.-G., Du, K.-Q., 2017. Concurrent ROS1 gene rearrangement and KRAS mutation in lung adenocarcinoma: A case report and literature review. Thorac Cancer 30, 863. doi: 10.1111/1759-7714.12518

### EQUIVALENTS

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention. All of the U.S. patents, U.S. patent application publications, U.S. patent applications, foreign patents, foreign patent application and non-patent publications referred to in this specification and/or listed in the Application Data Sheet are incorporated herein by reference, in their entirety. Aspects of the embodiments can be modified, if necessary to employ concepts of the various patents, application and publications to provide yet further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for identifying whether a subject has a cancer that is sensitive to SHP2 inhibition, the method comprising determining whether the cancer comprises one or more cells containing an oncogenic tyrosine kinase fusion that causes MAPK activation, and, if so, identifying the subject as having a cancer that is sensitive to SHP2 inhibition.

2. (canceled)

3. A method for killing cancer cells with a SHP2 inhibitor, the method comprising the steps of:
a. determining whether the cancer cells contain an oncogenic tyrosine kinase fusion that causes MAPK activation; and
b. contacting the cancer cells with the SHP2 inhibitor if the cancer cells contain an oncogenic tyrosine kinase fusion that causes MAPK activation.

4. A method for treating a patient with a SHP2 inhibitor, wherein the patient has cancer, the method comprising the steps of:
a. determining whether the patient has a SHP2-sensitive cancer by:
i. obtaining or having obtained a biological sample from the patient; and
ii. performing or having performed an assay on the biological sample to determine if the patient has a tumor comprising one or more cells that contain an oncogenic tyrosine kinase fusion that causes MAPK activation; and
b. administering the SHP2 inhibitor to the patient if the patient has a tumor comprising one or more cells containing an oncogenic tyrosine kinase fusion that causes MAPK activation.

5. The method of claim 4, wherein the SHP2 inhibitor is selected from (i) NSC-87877; (ii) TN0155, (iii) of any one of Formula I, of Formula II, of Formula III, of Formula I-V1, of Formula I-V2, of Formula I-W, of Formula I-X, of Formula I-Y, of Formula I-Z, of Formula IV, of Formula V, of Formula VI, of Formula IV-X, of Formula IV-Y, of Formula IV-Z, of Formula VII, of Formula VIII, of Formula IX, and of Formula X disclosed herein; (iv) Compound C; (v) a SHP2 inhibitor listed on Table 1; (vi) a SHP2 inhibitor listed on Table 2; (vii) a pharmaceutically acceptable salt prodrug, solvate, hydrate, tautomer, or stereoisomer of any one of (i)-(vi), and (vii) combinations thereof.

6. (canceled)

7. The method of claim 4, wherein the oncogenic tyrosine kinase fusion is selected from a ROS1 fusions, an ALK fusion, a RET fusion, an NTRK1 fusion, an NTRK2 fusion, and an NTRK3 fusion.

8. The method of claim 4, wherein the oncogenic tyrosine kinase fusion is a SDC4-ROS1 fusion or an SLC34A2-ROS1 fusion.

9. The method of claim 4, wherein the oncogenic tyrosine kinase fusion is selected from a FIG-ROS1 fusion; a LRIG3-ROS1 fusion; an EZR-ROS1 fusion, and a TPM3-ROS1 fusion.

10. The method of claim 4, wherein the oncogenic tyrosine kinase fusion is selected from an EML4-ALK fusion.

11. The method of claim 4, wherein the MAPK activation is detected by measuring increased ERK phosphorylation.

12. The method of claim 4, wherein determining whether the cancer cells contain an oncogenic tyrosine kinase fusion that causes MAPK activation is achieved by genotyping a cell or cells in a biological sample obtained from the patient.

13. The method of claim 12, wherein the genotyping determines whether the cancer comprises a cell containing an oncogenic tyrosine kinase fusion selected from EML4-ALK, SDC4-ROS1 and SLC34A2-ROS1.

14. The method of claim 4, wherein if the cancer does not comprise any cells containing an oncogenic tyrosine kinase fusion that causes MAPK activation, then the method comprises administering a cancer therapy selected from chemotherapy, radiation therapy, and/or surgical tumor resection.

15. A method for treating a subject having a tumor with a SHP2 inhibitor, the method comprising:
a. determining whether a biological sample obtained from the subject contains an oncogenic tyrosine kinase fusion protein comprising a N-terminal fusion partner that causes the fusion protein to localize in an endosome; and
b. administering to the subject an inhibitor of SHP2 if the biological sample contains an oncogenic tyrosine kinase fusion protein comprising a N-terminal fusion partner that causes the fusion protein to localize in an endosome.

16. The method of claim 15, wherein the oncogenic tyrosine kinase fusion protein causes MAPK activation.

17. The method of claim 15, wherein the method further comprises administering a cancer therapy selected from chemotherapy, radiation therapy, and/or surgical tumor resection.

18. The method of claim 15, wherein the method further comprises administering an additional therapeutic agent.

19. The method of claim 3, wherein the contacting occurs in vivo in a subject.

20. The method of claim 19, wherein the contacting occurs via administration of the SHP2 inhibitor to the subject.

21. The method of claim 20, wherein the method further comprises administering a cancer therapy selected from chemotherapy, radiation therapy, and/or surgical tumor resection.

22. The method of claim 20, wherein the method further comprises administering an additional therapeutic agent.

23. The method of claim 18, wherein the additional therapeutic agent is selected from a TKI, a MAPK pathway inhibitor, an EGFR inhibitor, an ALK inhibitor, and a MEK inhibitor.

24. The method of claim 18, wherein the additional therapeutic agent is (i) a pharmaceutically acceptable salt, prodrug, solvate, hydrate, tautomer, or stereoisomer of a TKI, a MAPK pathway inhibitor, an EGFR inhibitor, an ALK inhibitor, or a MEK inhibitor, or (ii) a combination of any two or more of such pharmaceutically acceptable salts, prodrugs, solvates, hydrates, tautomers, or stereoisomers recited in (i).

25-28. (canceled)

29. The method of any one of claims 1, 3, 4 or 15, wherein the SHP2 inhibitor is 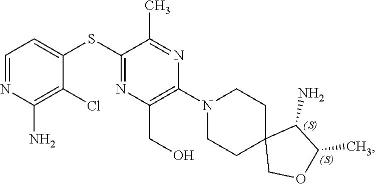
or a pharmaceutically acceptable salt, prodrug, solvate, hydrate, tautomer, or stereoisomer thereof.

30. The method of claim 29, wherein the SHP2 inhibitor is [chemical structure]
